SEQUENCE LISTING (101 sequences)

SEQ ID NO: 1 - DNA, 20 bp, Artificial sequence
  Synthetic oligonucleotide
acggcattggtgcacagttt

SEQ ID NO: 2 - DNA, 20 bp, Artificial sequence
  Synthetic oligonucleotide
tcttggttacatgaaatccc

SEQ ID NO: 3 - DNA, 20 bp, Artificial sequence
  Synthetic oligonucleotide
tgctccgttggtgcttgttc

SEQ ID NO: 4 - DNA, 20 bp, Artificial sequence
  Synthetic oligonucleotide
ccagctcaacccttctttaa

SEQ ID NO: 5 - DNA, 20 bp, Artificial sequence
  Synthetic oligonucleotide
aatggtttattccatggcca

SEQ ID NO: 6 - DNA, 17 bp, Artificial sequence
  Synthetic oligonucleotide
ggttcccgaggtgccca

SEQ ID NO: 7 - DNA, 20 bp, Artificial sequence
  Synthetic oligonucleotide
tcacagaattatcagcagta

SEQ ID NO: 8 - DNA, 16 bp, Artificial sequence
  Synthetic oligonucleotide
ggacacccacgccccc

SEQ ID NO: 9 - DNA, 18 bp, Artificial sequence
  Synthetic oligonucleotide
tcactttcataatgctgg

SEQ ID NO: 10 - DNA, 20 bp, Artificial sequence
  Synthetic oligonucleotide
gcacactcagcaggaccccc

SEQ ID NO: 11 - DNA, 20 bp, Artificial sequence
  Synthetic oligonucleotide
gcctcagtctgcttcgcacc

SEQ ID NO: 12 - DNA, 20 bp, Artificial sequence
  Synthetic oligonucleotide
agcatagttaacgagctccc

SEQ ID NO: 13 - DNA, 20 bp, Artificial sequence
  Synthetic oligonucleotide
agcttcttgtccagctttat

SEQ ID NO: 14 - DNA, 20 bp, Artificial sequence
  Synthetic oligonucleotide
gcagccatggtgatcaggag

SEQ ID NO: 15 - DNA, 16 bp, Artificial sequence
  Synthetic oligonucleotide
ctatttggatgtcagc

SEQ ID NO: 16 - DNA, 20 bp, Artificial sequence
  Synthetic oligonucleotide
tgtcatattcctggatcctt

SEQ ID NO: 17 - DNA, 20 bp, Artificial sequence
  Synthetic oligonucleotide
ccgtcgcccttcagcacgca

SEQ ID NO: 18 - DNA, 20 bp, Artificial sequence
  Synthetic oligonucleotide
tcagggcattctttccattc

SEQ ID NO: 19 - DNA, 21 bp, Artificial sequence
  Synthetic oligonucleotide
cagcagcagagtcttcatcat

SEQ ID NO: 20 - DNA, 20 bp, Artificial sequence
  Synthetic oligonucleotide
gggacgcggcgctcggtcat

SEQ ID NO: 21 - DNA, 21 bp, Artificial sequence
  Synthetic oligonucleotide
gcgtttgctcttcttcttgcg

SEQ ID NO: 22 - DNA, 20 bp, Artificial sequence
  Synthetic oligonucleotide
gcccaagctggcatccgtca

SEQ ID NO: 23 - DNA, 20 bp, Artificial sequence
  Synthetic oligonucleotide
ctgagtctgttttccattct

SEQ ID NO: 24 - DNA, 20 bp, Artificial sequence
  Synthetic oligonucleotide
gtttgacatggcacaatgtt

SEQ ID NO: 25 - DNA, 20 bp, Artificial sequence
  Synthetic oligonucleotide
gtttgacatggcacaatgtt

SEQ ID NO: 26 - DNA, 20 bp, Artificial sequence
  Primer
tgacaacgacaccgtgtcct

SEQ ID NO: 27 - DNA, 19 bp, Artificial sequence
  Primer
atgcgacttgtcaggctgg

SEQ ID NO: 28 - DNA, 23 bp, Artificial sequence
  Probe
cgtggagaaccgcagcctccatt

SEQ ID NO: 29 - DNA, 19 bp, Artificial sequence
  Primer
gccacaggctcccagacat

SEQ ID NO: 30 - DNA, 25 bp, Artificial sequence
  Primer
tccatcctcttgatatctccttttg

SEQ ID NO: 31 - DNA, 31 bp, Artificial sequence
  Probe
acagccatcatcaaagagatcgttagcagaa

SEQ ID NO: 32 - DNA, 23 bp, Artificial sequence
  Primer
tgggttagagaaggcgtgtactg

SEQ ID NO: 33 - DNA, 18 bp, Artificial sequence
  Primer
tcagcggcaactgggaaa

SEQ ID NO: 34 - DNA, 25 bp, Artificial sequence
  Probe
cgttggcacgacaccttcagggact

SEQ ID NO: 35 - DNA, 21 bp, Artificial sequence
  Primer
cttcgatctggagtccgctta

SEQ ID NO: 36 - DNA, 21 bp, Artificial sequence
  Primer
ttcctgtctcggtgaggctta

SEQ ID NO: 37 - DNA, 25 bp, Artificial sequence
  Probe
cgttggcacgacaccttcagggact

SEQ ID NO: 38 - DNA, 20 bp, Artificial sequence
  Synthetic oligonucleotide
gcttcagtcatgacttcctt

SEQ ID NO: 39 - RNA, 21 nt, Artificial sequence
  Synthetic oligonucleotide
ucggaaggcagcuuuguacuu

SEQ ID NO: 40 - RNA, 21 nt, Artificial sequence
  Synthetic oligonucleotide
guacaaagcugccuuccgauu

SEQ ID NO: 41 - RNA, 21 nt, Artificial sequence
  Synthetic oligonucleotide
gaaguaaagcucuacaagauu

SEQ ID NO: 42 - RNA, 21 nt, Artificial sequence
  Synthetic oligonucleotide
ucuuguagagcuuuacuuccu

SEQ ID NO: 43 - RNA, 21 nt, Artificial sequence
  Synthetic oligonucleotide
uacauaaccggacauaaucuu

SEQ ID NO: 44 - RNA, 21 nt, Artificial sequence
  Synthetic oligonucleotide
gauuauguccgguuauguauu

SEQ ID NO: 45 - DNA, 20 bp, Artificial sequence
  Synthetic oligonucleotide
ctgctagcctctggatttga

SEQ ID NO: 46 - DNA, 20 bp, Artificial sequence
  Synthetic oligonucleotide
cggtgcaaggcttaggaatt

SEQ ID NO: 47 - RNA, 21 nt, Artificial sequence
  Synthetic oligonucleotide
gguuaccagauaccuguguuu

SEQ ID NO: 48 - RNA, 21 nt, Artificial sequence
  Synthetic oligonucleotide
acacagguaucugguaaccuu

SEQ ID NO: 49 - RNA, 21 nt, Artificial sequence
  Synthetic oligonucleotide
ggcaaaccguuuuagauaauu

SEQ ID NO: 50 - RNA, 21 nt, Artificial sequence
  Synthetic oligonucleotide
uuaucuaaaacgguuugccuu

SEQ ID NO: 51 - RNA, 21 nt, Artificial sequence
  Synthetic oligonucleotide
uacuucuugaucuaagcgguu

SEQ ID NO: 52 - RNA, 21 nt, Artificial sequence
  Synthetic oligonucleotide
ccgcuuagaucaagaaguauu

SEQ ID NO: 53 - RNA, 21 nt, Artificial sequence
  Synthetic oligonucleotide
uaacgcacugggauuguacuu

SEQ ID NO: 54 - RNA, 21 nt, Artificial sequence
  Synthetic oligonucleotide
guacaaucccagugcguuauu

SEQ ID NO: 55 - DNA, 21 bp, Artificial sequence
  Synthetic oligonucleotide
acacggaggugaccaacaatt

SEQ ID NO: 56 - RNA, 21 nt, Artificial sequence
  Synthetic oligonucleotide
uuguuggucaccuccguguuu

SEQ ID NO: 57 - DNA, 21 bp, Artificial sequence
  Synthetic oligonucleotide
gaaaaugcaucgcuagcaatt

SEQ ID NO: 58 - DNA, 21 bp, Artificial sequence
  Synthetic oligonucleotide
uugcuagcgaugcauuuuctt

SEQ ID NO: 59 - DNA, 21 bp, Artificial sequence
  Synthetic oligonucleotide
gcaaaucauccauaugcautt

SEQ ID NO: 60 - DNA, 21 bp, Artificial sequence
  Synthetic oligonucleotide
augcauauggaugauuugctt

SEQ ID NO: 61 - DNA, 21 bp, Artificial sequence
  Synthetic oligonucleotide
ggccgucucuauuggguugtt

SEQ ID NO: 62 - DNA, 21 bp, Artificial sequence
  Synthetic oligonucleotide
caacccaauagagacggcctt

SEQ ID NO: 63 - DNA, 16 bp, Artificial sequence
  Synthetic oligonucleotide
ttcagtcatgacttcc

SEQ ID NO: 64 - DNA, 20 bp, Artificial sequence
  Synthetic oligonucleotide
ccttccctgaaggttcctcc

SEQ ID NO: 65 - DNA, 20 bp, Artificial sequence
  Synthetic oligonucleotide
atcctgacaatataggcatc

SEQ ID NO: 66 - DNA, 21 bp, Artificial sequence
  Synthetic oligonucleotide
ggacaagcuggcacagauatt

SEQ ID NO: 67 - DNA, 21 bp, Artificial sequence
  Synthetic oligonucleotide
uaucugugccagcuugucctt

SEQ ID NO: 68 - DNA, 21 bp, Artificial sequence
  Synthetic oligonucleotide
ccugacgaucaaaucacugtt

SEQ ID NO: 69 - DNA, 21 bp, Artificial sequence
  Synthetic oligonucleotide
cagugauuugaucgucaggtc

SEQ ID NO: 70 - DNA, 21 bp, Artificial sequence
  Synthetic oligonucleotide
ggaguauaacuauggcuuctt

SEQ ID NO: 71 - DNA, 21 bp, Artificial sequence
  Synthetic oligonucleotide
gaagccauaguuauacucctt

SEQ ID NO: 72 - DNA, 21 bp, Artificial sequence
  Synthetic oligonucleotide
gaauaagucuagcuuccugtt

SEQ ID NO: 73 - DNA, 20 bp, Artificial sequence
  Synthetic oligonucleotide
caggaacuagacuuauuctt

SEQ ID NO: 74 - DNA, 21 bp, Artificial sequence
  Synthetic oligonucleotide
gaauaacucuguguuuacutt

SEQ ID NO: 75 - DNA, 21 bp, Artificial sequence
  Synthetic oligonucleotide
aguaaacacagaguuauuctt

SEQ ID NO: 76 - DNA, 21 bp, Artificial sequence
  Synthetic oligonucleotide
gaaagggaaccucgaguggtt

SEQ ID NO: 77 - DNA, 21 bp, Artificial sequence
  Synthetic oligonucleotide
ccacucgagguucccuuuctt

SEQ ID NO: 78 - DNA, 21 bp, Artificial sequence
  Synthetic oligonucleotide
ucaacaacgucaagcuacatt

SEQ ID NO: 79 - DNA, 21 bp, Artificial sequence
  Synthetic oligonucleotide
uguagcuugacguuguugatt

SEQ ID NO: 80 - DNA, 21 bp, Artificial sequence
  Synthetic oligonucleotide
uuauuagcgauugauuuggtt

SEQ ID NO: 81 - DNA, 21 bp, Artificial sequence
  Synthetic oligonucleotide
ccaaaucaaucgcuaauaatt

SEQ ID NO: 82 - DNA, 21 bp, Artificial sequence
  Synthetic oligonucleotide
uucugauaaacgccuguaatt

SEQ ID NO: 83 - DNA, 21 bp, Artificial sequence
  Synthetic oligonucleotide
uuacaggcguuuaucagaatt

SEQ ID NO: 84 - DNA, 21 bp, Artificial sequence
  Synthetic oligonucleotide
uaaauucccacacuuauuctt

SEQ ID NO: 85 - DNA, 21 bp, Artificial sequence
  Synthetic oligonucleotide
gaauaagugugggaauuuatt

SEQ ID NO: 86 - DNA, 21 bp, Artificial sequence
  Synthetic oligonucleotide
gcacagguguaacgaguaatt

SEQ ID NO: 87 - DNA, 21 bp, Artificial sequence
  Synthetic oligonucleotide
uuacucguuacaccugugctt

SEQ ID NO: 88 - DNA, 21 bp, Artificial sequence
  Synthetic oligonucleotide
ggagagguuucguagcuuatt

SEQ ID NO: 89 - DNA, 21 bp, Artificial sequence
  Synthetic oligonucleotide
uaagcuacgaaaccucucctg

SEQ ID NO: 90 - DNA, 21 bp, Artificial sequence
  Synthetic oligonucleotide
cagagcuucuugaaugucatt

SEQ ID NO: 91 - DNA, 21 bp, Artificial sequence
  Synthetic oligonucleotide
ucacauucaagaagcucugtt

SEQ ID NO: 92 - DNA, 21 bp, Artificial sequence
  Synthetic oligonucleotide
gaagaguccagauuuaucctt

SEQ ID NO: 93 - DNA, 21 bp, Artificial sequence
  Synthetic oligonucleotide
ggauaaaucuggacucuuctt

SEQ ID NO: 94 - DNA, 21 bp, Artificial sequence
  Synthetic oligonucleotide
ccaaucuccaguugcuuuctt

SEQ ID NO: 95 - DNA, 21 bp, Artificial sequence
  Synthetic oligonucleotide
gaaagcaacuggagauuggtt

SEQ ID NO: 96 - DNA, 21 bp, Artificial sequence
  Synthetic oligonucleotide
uuggucguagguucuucuctt

SEQ ID NO: 97 - DNA, 21 bp, Artificial sequence
  Synthetic oligonucleotide
gagaagaaccuacgaccaatt

SEQ ID NO: 98 - DNA, 21 bp, Artificial sequence
  Synthetic oligonucleotide
uuccguacaguuucuuauutt

SEQ ID NO: 99 - DNA, 21 bp, Artificial sequence
  Synthetic oligonucleotide
aauaagaaacuguacggaatt

SEQ ID NO: 100 - DNA, 21 bp, Artificial sequence
  Synthetic oligonucleotide
gaucggaggcuuauucauctt

SEQ ID NO: 101 - DNA, 21 bp, Artificial sequence
  Synthetic oligonucleotide
gaugaauaagccuccgauctt

(19) United States
(12) Patent Application Publication
(10) Pub. No.: US 2015/0322428 A1
(43) Pub. Date: Nov. 12, 2015

(54) COMPOUNDS AND METHODS FOR IMPROVED CELLULAR UPTAKE OF ANTISENSE COMPOUNDS

(71) Applicant: Isis Pharmaceuticals, Inc., Carlsbad, CA (US)

(72) Inventors: Erich Koller, Basel (CH); C. Frank Bennett, Carlsbad, CA (US)

(73) Assignee: Isis Pharmaceuticals, Inc., Carlsbad, CA (US)

(21) Appl. No.: 14/409,332

(22) PCT Filed: Jun. 18, 2013
(86) PCT No.: PCT/US13/46421

§ 371 Date: Dec. 18, 2014

Related U.S. Application Data
(60) Provisional application No. 61/661,215, filed on Jun. 18, 2012.
(60) Provisional application No. 61/823,324, filed on May 14, 2013.

(51) Int. Cl. C12N 15/113

(52) U.S. Cl. CPC C12N 15/113; C12N 2310/11; C12N 2310/14; C12N 2310/341; C12N 2310/315; C12N 2310/31; C12N 2310/321; C12N 2310/3341; C12N 2320/31; C12N 2320/50

(57) ABSTRACT

The present invention provides method of increasing the efficacy and potency of antisense compounds. In certain embodiments, the invention provides methods for improved cellular uptake. In certain embodiments, the resulting antisense activity is greater at a particular concentration of antisense compound than the antisense activity at the same concentration of the antisense compound in the absence of the ESCRT modulator.

### SEQUENCE LISTING

The present application is being filed along with a Sequence Listing in electronic format. The Sequence Listing is provided as a file entitled CORE0106WOSEQ.txt, created Jun. 18, 2013, which is 24 Kb in size. The information in the electronic format of the sequence listing is incorporated herein by reference in its entirety.

### BACKGROUND OF THE INVENTION

Antisense compounds have been used to modulate target nucleic acids. Antisense compounds comprising a variety of chemical modifications and motifs have been reported. In certain instances, such compounds are useful as research tools, diagnostic reagents, and as therapeutic agents. Certain DNA-like oligomeric compounds have been shown to reduce protein expression. Certain RNA-like compounds are known to inhibit protein expression in cells. Such RNA-like compounds function, at least in part, through the RNA-inducing silencing complex (RISC). RNA-like compounds may be single-stranded or double-stranded. Antisense compounds have also been shown to alter processing of pre-mRNA and to modulate non-coding RNA molecules. In certain instances antisense compounds have been shown to modulate protein expression by binding to a target messenger RNA (mRNA) encoding the protein. In certain instances, such binding of an antisense compound to its target mRNA results in cleavage of the mRNA. Antisense compounds that modulate processing of a pre-mRNA have also been reported. Such antisense compounds alter splicing, interfere with polyadenlyation or prevent formation of the 5′-cap of a pre-mRNA. Compositions and methods that increase productive uptake of antisense compounds in cells are desired. Compositions and methods that facilitate the manufacture, storage, administration, and delivery of antisense compounds are also desired.

### SUMMARY OF THE INVENTION

The present disclosure provides compounds and methods for modulating a target nucleic acid in a cell. In certain embodiments, the cell is sensitized for antisense activity. In certain embodiments, the cell is sensitized by contact with an ESCRT modulator. In certain such embodiments, the cell is contacted with an ESCRT modulator and an antisense compounds. In certain embodiments, the resulting antisense activity is greater at a particular concentration of antisense compound than the antisense activity at the same concentration of the antisense compound in the absence of the ESCRT modulator.

The present disclosure provides the following non-limiting numbered embodiments:

We claim:

### Embodiment 1

A method of sensitizing a cell for antisense modulation comprising, reducing the amount or activity of at least one protein or nucleic acid transcript; and thereby sensitizing the cell for antisense modulation.

### Embodiment 2

The method of embodiment 1 comprising contacting the cell with at least one protein or nucleic acid transcript modulator.

### Embodiment 3

The method of embodiment 1 or 2, wherein at least one protein or nucleic acid transcript modulator is a Lip5 modulator.

### Embodiment 4

The method of embodiment 1 or 2, wherein at least one protein or nucleic acid transcript modulator is a Lip5 modulator.

### Embodiment 5

The method of embodiment 1 or 2, wherein at least one protein or nucleic acid transcript modulator is a Rab27A modulator.

### Embodiment 6

The method of embodiment 1 or 2, wherein at least one protein or nucleic acid transcript modulator is a Rab27B modulator.

### Embodiment 7

The method of embodiment 1 or 2, wherein at least one protein or nucleic acid transcript modulator is a SYTL4 modulator.

### Embodiment 8

The method of embodiment 1 or 2, wherein at least one protein or nucleic acid transcript modulator is a SLAC2B modulator.

### Embodiment 9

The method of embodiment 1 or 2, wherein at least one protein or nucleic acid transcript modulator is a AP2M1 modulator.

### Embodiment 10

The method of any of embodiments 1 to 9, wherein at least one protein or nucleic acid transcript modulator is an ESCRT modulator.

### Embodiment 11

A method of sensitizing a cell for antisense modulation comprising, reducing the amount or activity of at least one ESCRT associated nucleic acid transcript; and thereby sensitizing the cell for antisense modulation.

### Embodiment 12

A method of sensitizing a cell for antisense modulation comprising, reducing the amount or activity of at least one ESCRT associated protein; and thereby sensitizing the cell for antisense modulation.

### Embodiment 13

The method of embodiment 11 or 12 comprising contacting the cell with at least one ESCRT modulator.

### Embodiment 14

The method of embodiment 13, wherein at least one ESCRT modulator is an ESCRT-I modulator.

### Embodiment 15

The method of embodiment 13-14, wherein at least one ESCRT modulator is a Vps28 modulator.

### Embodiment 16

The method of embodiment 13-15, wherein at least one ESCRT modulator is a Tsg101 modulator.

### Embodiment 17

The method of any of embodiments 13-16, wherein at least one ESCRT modulator is a Vps37 modulator.

### Embodiment 18

The method of any of embodiments 13-17, wherein at least one ESCRT modulator is an Mvb12 modulator.

### Embodiment 19

The method of embodiment 18, wherein at least one ESCRT modulator is an Mvb12a modulator.

### Embodiment 20

The method of embodiment 18, wherein at least one ESCRT modulator is an Mvb12b modulator.

### Embodiment 21

The method of any of embodiments 13-20, wherein at least one ESCRT modulator is an Hrs modulator.

### Embodiment 22

The method of any of embodiments 13-21, wherein at least one ESCRT modulator is an Alix modulator.

### Embodiment 23

The method of any of embodiments 13-22, wherein at least one ESCRT modulator is an ESCRT-II modulator.

### Embodiment 24

The method of any of embodiments 13-22, wherein at least one ESCRT modulator is Vps4 modulator.

### Embodiment 25

The method of any of embodiments 13-24, wherein at least one ESCRT modulator is selected from among: a Vps22 modulator, a Vps36 modulator, a Vps4, and a Vps25 modulator.

### Embodiment 26

The method of any of embodiments 13-24, wherein at least one ESCRT modulator is an ESCRT-III modulator.

### Embodiment 27

The method of any of embodiments 13-26, wherein at least one ESCRT modulator is selected from among: a Vps20 modulator, a Vps32 modulator, a Vps24 modulator, a Vps2 modulator, a Vps4 modulator, a Vta1 modulator, a Vps60 modulator, a lst1 modulator, a Did2 modulator, and a DUBs modulator.

### Embodiment 28

The method of any of embodiments 13-27, wherein at least one ESCRT modulator is an ESCRT-0 modulator.

### Embodiment 29

The method of any of embodiments 13-27, wherein at least one ESCRT modulator is selected from among: an Eps15b modulator, a CB modulator, a STAM modulator, a UIM modulator, a FYVE modulator, a Clathrin modulator, a PSAP modulator, and a Ptdlns(3)P modulator.

### Embodiment 30

The method of any of embodiments 1-29, wherein at least one ESCRT modulator is an antisense compound targeting an ESCRT transcript.

### Embodiment 31

The method of embodiment 30, wherein the antisense compound targeting an ESCRT transcript is single-stranded.

### Embodiment 32

The method of embodiment 30, wherein the antisense compound targeting an ESCRT transcript is double-stranded.

### Embodiment 33

The method of embodiment 31 or 32, wherein the antisense compound targeting an ESCRT transcript is an RNAi compound.

### Embodiment 34

The method of embodiment 31, wherein the antisense compound targeting an ESCRT transcript is an RNase H antisense compound.

### Embodiment 35

The method of any of embodiments 1-29, wherein at least one ESCRT modulator is an antibody.

### Embodiment 36

The method of embodiment 35, wherein the antibody is monoclonal.

### Embodiment 37

The method of any of embodiments 1-29, wherein at least one ESCRT modulator is a small molecule.

### Embodiment 38

The method of any of embodiments 1-37 comprising contacting the cell with at least one non-ESCRT antisense compound, wherein the non-ESCRT antisense compound is complementary to a target nucleic acid other than an ESCRT transcript.

### Embodiment 39

The method of embodiment 38, wherein the non-ESCRT antisense compound comprises an antisense oligonucleotide.

### Embodiment 40

The method of embodiment 39, wherein the antisense oligonucleotide comprises at least one modified nucleoside.

### Embodiment 41

The method of embodiment 40, wherein at least one modified nucleoside comprises a modified sugar moiety.

### Embodiment 42

The method of embodiment 41, wherein at least one modified sugar moiety is a 2′-substituted sugar moiety.

### Embodiment 43

The method of embodiment 42, wherein the 2′-substitutent of at least one 2′-substituted sugar moiety is selected from among: 2′-OMe, 2′-F, and 2′-MOE.

### Embodiment 44

The method of embodiment 43, wherein the 2′-substituent of at least one 2′-substituted sugar moiety is a 2′-MOE.

### Embodiment 45

The method of any of embodiments 40-44, wherein at least one modified sugar moiety is a bicyclic sugar moiety.

### Embodiment 46

The method of embodiment 45, wherein at least one bicyclic sugar moiety is LNA or cEt.

### Embodiment 47

The method of any of embodiments 41-46, wherein at least one modified sugar moiety is a sugar surrogate.

### Embodiment 48

The method of embodiment 47, wherein at least one sugar surrogate is a morpholino.

### Embodiment 49

The method of embodiment 48, wherein at least one sugar surrogate is a modified morpholino.

### Embodiment 50

The method of any of embodiments 39-49, wherein the antisense oligonucleotide comprises at least one modified internucleoside linkage.

### Embodiment 51

The method of embodiment 50, wherein each internucleoside linkage is a modified internucleoside linkage.

### Embodiment 52

The method of embodiment 50 or 51, wherein the antisense oligonucleotide comprises at least one phosphorothioate internucleoside linkage.

### Embodiment 53

The method of embodiments 39-49, wherein the antisense oligonucleotide comprises at least one unmodified internucleoside linkage.

### Embodiment 54

The method of embodiment 53, wherein each internucleoside linkage is an unmodified internucleoside linkage.

### Embodiment 55

The method of embodiment 53 or 54, wherein the antisense oligonucleotide comprises at least one phosphodiester internucleoside linkage.

### Embodiment 56

The method of any of embodiments 38-55, wherein the antisense compound complementary to a target nucleic acid other than an ESCRT transcript comprises at least one conjugate.

### Embodiment 57

The method of any of embodiments 38-56, wherein the non-ESCRT antisense compound is single-stranded.

### Embodiment 58

The method of any of embodiments 38-56, wherein the non-ESCRT antisense compound is double-stranded.

### Embodiment 59

The method of any of embodiments 38-58, wherein the non-ESCRT antisense compound is an RNAi compound.

### Embodiment 60

The method of any of embodiments 38-59, wherein the non-ESCRT antisense compound is an RNase H antisense compound.

### Embodiment 61

The method of any of embodiments 1-60, wherein the cell is in vitro.

### Embodiment 62

The method of any of embodiments 1-60, wherein the cell is in an animal.

### Embodiment 63

The method of embodiment 62, wherein the animal is a human.

### Embodiment 64

A method for reducing the amount or activity of a target nucleic acid in a cell comprising contacting a cell with an ESCRT modulator and an antisense compound complementary to the target nucleic acid, wherein the target nucleic acid is other than an ESCRT transcript; and thereby reducing the amount or activity of the target nucleic acid in the cell.

### Embodiment 65

The method of embodiment 64, wherein the ESCRT modulator is the ESCRT modulator according to any of embodiments 1-37.

### Embodiment 66

The method of embodiment 64 or 65, wherein the antisense compound complementary to a target nucleic acid is the non-ESCRT antisense compound according to any of embodiments 24-45.

### Embodiment 67

The method of any of embodiments 64-66, wherein the cell is in vitro.

### Embodiment 68

The method of any of embodiments 64-66, wherein the cell is in an animal.

### Embodiment 69

The method of embodiment 68, wherein the animal is a human.

### Embodiment 70

A method of reducing the amount or activity of a target nucleic acid in a cell in an animal comprising administering to the animal an ESCRT modulator and an antisense compound complementary to the target nucleic acid, wherein the target nucleic acid is other than an ESCRT transcript; and thereby reducing the amount or activity of the target nucleic acid in a cell of the animal.

### Embodiment 71

The method of embodiment 70, wherein the ESCRT modulator is the ESCRT modulator according to any of embodiments 1-37.

### Embodiment 72

The method of embodiment 70 or 71, wherein the antisense compound complementary to a target nucleic acid is the non-ESCRT antisense compound according to any of embodiments 24-45.

### Embodiment 73

The method of any of embodiments 70-72, wherein the potency of the antisense compound complementary to the target nucleic acid is improved relative to the potency of the same antisense compound when administered without the ESCRT modulator.

### Embodiment 74

The method of embodiment 73, wherein the potency is improved at least two-fold as measured by ED₅₀.

### Embodiment 75

The method of embodiment 73, wherein the potency is improved at least five-fold as measured by ED₅₀.

### Embodiment 76

The method of embodiment 73, wherein the potency is improved at least ten-fold as measured by ED₅₀.

### Embodiment 77

The method of any of embodiments 70-76, wherein the animal is a human.

### Embodiment 78

The method of any of embodiments 70-77, wherein the antisense compound complementary to the target nucleic acid is at least 80% complementary to the target nucleic acid.

### Embodiment 79

The method of embodiment 78, wherein the antisense compound complementary to the target nucleic acid is 100% complementary to the target nucleic acid.

### Embodiment 80

The method of any of embodiments 70-79, wherein the ESCRT modulator and the antisense compound complementary to the target nucleic acid are administered together.

### Embodiment 81

The method of any of embodiments 70-80, wherein the ESCRT modulator and the antisense compound complementary to the target nucleic acid are administered separately.

### Embodiment 82

The method of any of embodiments 38-81, wherein the antisense compound complementary to a target nucleic acid other than an ESCRT transcript is at least 80% complementary to the target nucleic acid other than an ESCRT transcript.

### Embodiment 83

The method of embodiment 82, wherein the antisense compound complementary to a target nucleic acid other than an ESCRT transcript is 100% complementary to the target nucleic acid other than an ESCRT transcript.

### Embodiment 84

The method of any of embodiments 64-69, wherein the antisense compound complementary to the target nucleic acid is at least 80% complementary to the target nucleic acid.

### Embodiment 85

The method of embodiment 83, wherein the antisense compound complementary to the target nucleic acid is 100% complementary to the target nucleic acid.

### Embodiment 86

The method of any of embodiments 38-85, wherein the target nucleic acid is an RNA.

### Embodiment 87

The method of any of embodiments 38-85, wherein the target nucleic acid is an mRNA.

### Embodiment 88

The method of any of embodiments 38-85, wherein the target nucleic acid is a pre-mRNA.

### Embodiment 89

The method of any of embodiments 38-85, wherein the target nucleic acid is a microRNA.

### Embodiment 90

The method of any of embodiments 38-85, wherein the target nucleic acid is a non-coding RNA.

### Embodiment 91

The method of any of embodiments 38-85, wherein the target nucleic acid is a promoter-directed RNA.

### Embodiment 92

The method of any of embodiments 38-85, wherein the target nucleic acid is long non-coding RNA.

### Embodiment 93

The method of any of embodiments 38-85, wherein the target nucleic acid is a long intergenic RNA.

### Embodiment 94

The method of any of embodiments 38-85, wherein the target nucleic acid is a natural antisense transcript.

### Embodiment 95

A pharmaceutical composition comprising an ESCRT modulator and a non-ESCRT antisense compound.

### Embodiment 96

The pharmaceutical composition of embodiment 85, wherein the ESCRT modulator is the ESCRT modulator according to any of embodiments 1-37.

### Embodiment 97

The pharmaceutical composition of embodiment 95 or 96, wherein the non-ESCRT antisense compound is the non-ESCRT antisense compound according to any of embodiments 38-94.

### Embodiment 98

The pharmaceutical composition of any of embodiments 95-87 comprising an excipient.

### Embodiment 99

A method of sensitizing a cell for antisense modulation comprising, increasing the amount or activity of LDL-R protein and/or LDL-R related protein; and thereby sensitizing the cell for antisense modulation.

### Embodiment 100

The method of embodiment 99 comprising contacting the cell with at least one LDL-R modulator.

### Embodiment 101

The method of embodiment 100, wherein the LDL-R modulator is not a statin.

### Embodiment 102

The method of any of embodiments 99-101, wherein at least one LDL-R modulator is an antisense compound targeting an ESCRT transcript.

### Embodiment 103

The method of any of embodiments 99-102 wherein at least one LDL-R modulator is an antisense compound targeting a PCSK9 transcript.

### Embodiment 104

The method of embodiment 102, wherein the ESCRT transcript is a Vps28 transcript.

### Embodiment 105

The method of embodiment 102 or 103, wherein the antisense compound targeting an ESCRT or PCSK9 transcript is single-stranded.

### Embodiment 106

The method of embodiment 102 or 103, wherein the antisense compound targeting an ESCRT or PCSK9 transcript is double-stranded.

### Embodiment 107

The method of embodiment 102 or 103, wherein the antisense compound targeting an ESCRT or PCSK9 transcript is an RNAi compound.

### Embodiment 108

The method of embodiment 102 or 103, wherein the antisense compound targeting an ESCRT or PCSK9 transcript is an RNase H antisense compound.

### Embodiment 109

The method of embodiment 100, wherein at least one LDL-R modulator is an antibody.

### Embodiment 110

The method of embodiment 109, wherein the antibody is monoclonal.

### Embodiment 111

The method of embodiment 100, wherein at least one LDL-R modulator is a small molecule.

### Embodiment 112

The method of any of embodiments 99 to 111 comprising contacting the cell with at least one non-LDL-R antisense compound, wherein the non-LDL-R antisense compound is complementary to a target nucleic acid other than an ESCRT transcript or a PCSK9 transcript.

### Embodiment 113

The method of embodiment 112, wherein the non-LDL-R antisense compound comprises an antisense oligonucleotide.

### Embodiment 114

The method of embodiment 113, wherein the antisense oligonucleotide comprises at least one modified nucleoside.

### Embodiment 115

The method of embodiment 114, wherein at least one modified nucleoside comprises a modified sugar moiety.

### Embodiment 116

The method of embodiment 115, wherein at least one modified sugar moiety is a 2′-substituted sugar moiety.

### Embodiment 117

The method of embodiment 116, wherein the 2′-substitutent of at least one 2′-substituted sugar moiety is selected from among: 2′-OMe, 2′-F, and 2′-MOE.

### Embodiment 118

The method of embodiment 117, wherein the 2′-substituent of at least one 2′-substituted sugar moiety is a 2′-MOE.

### Embodiment 119

The method of any of embodiments 112-118, wherein at least one modified sugar moiety is a bicyclic sugar moiety.

### Embodiment 120

The method of embodiment 118, wherein at least one bicyclic sugar moiety is LNA or cEt.

### Embodiment 121

The method of any of embodiments 112-120, wherein at least one modified sugar moiety is a sugar surrogate.

### Embodiment 122

The method of embodiment 121, wherein at least one sugar surrogate is a morpholino.

### Embodiment 123

The method of embodiment 121, wherein at least one sugar surrogate is a modified morpholino.

### Embodiment 124

The method of any of embodiments 12-123, wherein the antisense oligonucleotide comprises at least one modified internucleoside linkage.

### Embodiment 125

The method of embodiment 124, wherein each internucleoside linkage is a modified internucleoside linkage.

### Embodiment 126

The method of embodiment 124 or 125, wherein the antisense oligonucleotide comprises at least one phosphorothioate internucleoside linkage.

### Embodiment 127

The method of embodiments 112-123, wherein the antisense oligonucleotide comprises at least one unmodified internucleoside linkage.

### Embodiment 128

The method of embodiment 127, wherein each internucleoside linkage is an unmodified internucleoside linkage.

### Embodiment 129

The method of embodiment 127 or 128, wherein the antisense oligonucleotide comprises at least one phosphodiester internucleoside linkage.

### Embodiment 130

The method of any of embodiments 112-129, wherein the antisense compound complementary to a target nucleic acid other than an ESCRT transcript or PCSK9 comprises at least one conjugate.

### Embodiment 131

The method of any of embodiments 112-130, wherein the non-ESCRT or non-PCSK9 antisense compound is single-stranded.

### Embodiment 132

The method of any of embodiments 112-130, wherein the non-ESCRT or non-PCSK9 antisense compound is double-stranded.

### Embodiment 133

The method of any of embodiments 112-132, wherein the non-ESCRT or non-PCSK9 antisense compound is an RNAi compound.

### Embodiment 134

The method of any of embodiments 112-133, wherein the non-ESCRT or non-PCSK9 antisense compound is an RNase H antisense compound.

### Embodiment 135

The method of any of embodiments 112-134, wherein the cell is contacted with at least two non-LDL-R antisense compounds.

### Embodiment 136

The method of any of embodiments 100-135, wherein the cell is in vitro.

### Embodiment 137

The method of any of embodiments 100-135, wherein the cell is in an animal.

### Embodiment 138

The method of embodiment 137, wherein the animal is a human.

### Embodiment 139

A method for reducing the amount or activity of a target nucleic acid in a cell comprising contacting a cell with an LDL-R modulator and an antisense compound complementary to the target nucleic acid, wherein the target nucleic acid is other than an ESCRT transcript or a PCSK9 transcript; and thereby reducing the amount or activity of the target nucleic acid in the cell.

### Embodiment 140

The method of embodiment 139, wherein the LDL-R modulator is the LDL-R modulator according to any of embodiments 101-121.

### Embodiment 141

The method of embodiment 139 or 140, wherein the antisense compound complementary to a target nucleic acid is the non-ESCRT antisense compound or non PCSK-9 antisense compound according to any of embodiments 97-115.

### Embodiment 142

The method of any of embodiments 139-141, wherein the cell is in vitro.

### Embodiment 143

The method of any of embodiments 139-141, wherein the cell is in an animal.

### Embodiment 144

The method of embodiment 143, wherein the animal is a human.

### Embodiment 145

A method of reducing the amount or activity of a target nucleic acid in a cell in an animal comprising administering to the animal an LDL-R modulator and an antisense compound complementary to the target nucleic acid, wherein the target nucleic acid is other than an ESCRT transcript or other than a PCSK9 transcript; and thereby reducing the amount or activity of the target nucleic acid in a cell of the animal.

### Embodiment 146

The method of embodiment 145, wherein the LDL-R modulator is the LDL-R modulator according to any of embodiments 101-111.

### Embodiment 147

The method of embodiment 145-146, wherein the LDL-R modulator increases the amount of LDL-R.

### Embodiment 148

The method of any of embodiments 145-147, wherein the potency of the antisense compound complementary to the target nucleic acid is improved relative to the potency of the same antisense compound when administered without the LDL-R modulator.

### Embodiment 149

The method of any of embodiments 145-147, wherein the animal is a human.

### Embodiment 150

The method of any of embodiments 139-149, wherein the antisense compound complementary to the target nucleic acid is at least 80% complementary to the target nucleic acid.

### Embodiment 151

The method of embodiment 150, wherein the antisense compound complementary to the target nucleic acid is 100% complementary to the target nucleic acid.

### Embodiment 152

The method of any of embodiments 139-149, wherein the LDL-R modulator and the antisense compound complementary to the target nucleic acid are administered together.

### Embodiment 153

The method of any of embodiments 139-149, wherein the LDL-R modulator and the antisense compound complementary to the target nucleic acid are administered separately.

### Embodiment 154

The method of any of embodiments 139-149, wherein the antisense compound complementary to the target nucleic acid is at least 80% complementary to the target nucleic acid.

### Embodiment 155

The method of embodiment 154, wherein the antisense compound complementary to the target nucleic acid is 100% complementary to the target nucleic acid.

### Embodiment 156

The method of any of embodiments 99-155, wherein the target nucleic acid is an RNA.

### Embodiment 157

The method of any of embodiments 99-155, wherein the target nucleic acid is an mRNA.

### Embodiment 158

The method of any of embodiments 99-155, wherein the target nucleic acid is a pre-mRNA.

### Embodiment 159

The method of any of embodiments 99-155, wherein the target nucleic acid is a microRNA.

### Embodiment 160

The method of any of embodiments 99-155, wherein the target nucleic acid is a non-coding RNA.

### Embodiment 161

The method of any of embodiments 99-155, wherein the target nucleic acid is a promoter-directed RNA.

### Embodiment 162

The method of any of embodiments 99-155, wherein the target nucleic acid is long non-coding RNA.

### Embodiment 163

The method of any of embodiments 99-155, wherein the target nucleic acid is a long intergenic RNA.

### Embodiment 164

The method of any of embodiments 99-155, wherein the target nucleic acid is a natural antisense transcript.

### Embodiment 165

A pharmaceutical composition comprising an LDL-R modulator and a non-ESCRT antisense compound.

### Embodiment 166

The pharmaceutical composition of embodiment 95, wherein the LDL-R modulator is the LDL-R modulator according to any of embodiments 101-111.

### Embodiment 167

The pharmaceutical composition of embodiment 165 or 166, wherein the non-ESCRT or non-PCSK9 antisense compound is the non-ESCRT or non-PCSK9 antisense compound according to any of embodiments 98-121.

### Embodiment 168

The pharmaceutical composition of any of embodiments 165-167 comprising an excipient.

In certain embodiments, methods compounds and compositions of the present invention have therapeutic value. In certain such embodiments, the dose of antisense compound administered to a patient may be decreased when co-administered with an ESCRT modulator. Such co-administration may be at the same time and/or different times. In certain embodiments, for example, an ESCRT modulator is administered prior to administration with the antisense compound.

### BRIEF DESCRIPTION OF THE FIGURES

FIG. 1 illustrates a reduction in Mvb12b mRNA levels in MHT and bEND cells when treated with Mvb12b siRNA compared to negative control.

FIG. 2 illustrates a reduction in Vps37 mRNA levels in MHT and bEND cells when treated with Vps37 siRNA compared to negative control.

FIG. 3 illustrates a reduction in Tsg101 mRNA levels in MHT and bEND cells when treated with Tsg101 siRNAs compared to negative control.

FIG. 4 illustrates the inhibition of EGFR degradation in Vps28 or Tsg101 depleted cells compared to negative control in which Vps28 and Tsg101 were not depleted.

FIG. 5 illustrates the localization of ASO in the lysosomes in both negative control siRNA treated cells and Vps28 siRNA-3 treated cells. The lysosomes in Vps28 siRNA-3 treated cells are enlarged and are reminiscent to multivesicular bodies.

FIG. 6 illustrates an increase in vesicle size when MHT cells were treated with Vps28 siRNA-3 compared to negative control.

FIG. 7 illustrates an increase in ASO uptake into MHT cells when treated with Vps28 siRNA-3 compared to negative control.

### DETAILED DESCRIPTION OF THE INVENTION

Unless specific definitions are provided, the nomenclature used in connection with, and the procedures and techniques of, analytical chemistry, synthetic organic chemistry, and medicinal and pharmaceutical chemistry described herein are those well known and commonly used in the art. Standard techniques may be used for chemical synthesis, and chemical analysis. Certain such techniques and procedures may be found for example in “Carbohydrate Modifications in Antisense Research” Edited by Sangvi and Cook, American Chemical Society, Washington D.C., 1994; “Remington's Pharmaceutical Sciences,” Mack Publishing Co., Easton, Pa., 21^{st} edition, 2005; and “Antisense Drug Technology, Principles, Strategies, and Applications” Edited by Stanley T. Crooke, CRC Press, Boca Raton, Fla.; and Sambrook et al., “Molecular Cloning, A laboratory Manual,” 2^{nd} Edition, Cold Spring Harbor Laboratory Press, 1989, which are hereby incorporated by reference for any purpose. Where permitted, all patents, applications, published applications and other publications and other data referred to throughout in the disclosure are incorporated by reference herein in their entirety.

Unless otherwise indicated, the following terms have the following meanings:

As used herein, “ESCRT” or “Endosomal Sorting Complex Required for Transport (ESCRT)” means a complex involved in endosomal transport, as described in Raiborg & Stenmark, Nature, 2009, 458, 445-452.

As used herein, “ESCRT transcript” means a nucleic acid, the expression of which results in one or more ESCRT protein.

As used herein, “ESCRT protein” means a protein member of the ESCRT complex.

As used herein, “ESCRT modulator” means a compound capable of modulating the amount and/or activity of the ESCRT complex. In certain embodiments, an ESCRT modulator is selected from among an antisense compound complementary to an ESCRT transcript, an antibody directed to an ESCRT protein, and a small molecule that binds to a an ESCRT protein. In certain embodiments, an ESCRT modulator alters the amount and/or activity of ESCRT indirectly by binding to a non-ESCRT protein or nucleic acid. In certain embodiments, an ESCRT modulator is an ESCRT inhibitor, which results in a decrease in the amount and/or activity of ESCRT. In certain embodiments, ESCRT inhibition sensitizes a cell to the activity of one or more antisense compound. In certain embodiments, ESCRT inhibition sensitizes a cell to the activity of an oligonucleotide that are is not an antisense compound (e.g., aptamers, the activity of which do depend on hybridizization to a complementary nucleic acid). In certain embodiments, an ESCRT modulator is an ESCRT activator, which increases the amount and/or activity of ESCRT. In certain embodiments, ESCRT activators make cells more resistant to antisense compounds.

As used herein, “non-ESCRT antisense compound” means an antisense compound directed to a target other than an ESCRT transcript.

As used herein, “excipient” means any compound or composition other than water or an antisense oligonucleotide.

As used herein, “chemical modification” means a chemical difference in a compound when compared to a reference compound. In certain contexts, a chemical modification is a chemical difference when compared to a naturally occurring counterpart. In reference to an oligonucleotide, chemical modification does not include differences only in nucleobase sequence. Chemical modifications of oligonucleotides include nucleoside modifications (including sugar moiety modifications and nucleobase modifications) and internucleoside linkage modifications.

As used herein, “furanosyl” means a structure comprising a 5-membered ring comprising four carbon atoms and one oxygen atom.

As used herein, “naturally occurring sugar moiety” means a ribofuranosyl as found in naturally occurring RNA or a deoxyribofuranosyl as found in naturally occurring DNA.

As used herein, “sugar moiety” means a naturally occurring sugar moiety or a modified sugar moiety of a nucleoside. As used herein, “modified sugar moiety” means a substituted sugar moiety, a bicyclic or tricyclic sugar moiety, or a sugar surrogate.

As used herein, “substituted sugar moiety” means a furanosyl comprising at least one substituent group that differs from that of a naturally occurring sugar moiety. Substituted sugar moieties include, but are not limited to furanosyls comprising substituents at the 2′-position, the 3′-position, the 5′-position and/or the 4′-position.

As used herein, “2′-substituted sugar moiety” means a furanosyl comprising a substituent at the 2′-position other than H or OH. Unless otherwise indicated, a 2′-substituted sugar moiety is not a bicyclic sugar moiety (i.e., the 2′-substituent of a 2′-substituted sugar moiety does not form a bridge to another atom of the furanosyl ring.

As used herein, “MOE” means —OCH₂CH₂OCH₃.

As used herein, “bicyclic sugar moiety” means a modified sugar moiety comprising a 4 to 7 membered ring (including but not limited to a furanosyl) comprising a bridge connecting two atoms of the 4 to 7 membered ring to form a second ring, resulting in a bicyclic structure. In certain embodiments, the 4 to 7 membered ring is a sugar ring. In certain embodiments the 4 to 7 membered ring is a furanosyl. In certain such embodiments, the bridge connects the 2′-carbon and the 4′-carbon of the furanosyl.

As used herein the term “sugar surrogate” means a structure that does not comprise a furanosyl and that is capable of replacing the naturally occurring sugar moiety of a nucleoside, such that the resulting nucleoside is capable of (1) incorporation into an oligonucleotide and (2) hybridization to a complementary nucleoside. Such structures include rings comprising a different number of atoms than furanosyl (e.g., 4, 6, or 7-membered rings); replacement of the oxygen of a furanosyl with a non-oxygen atom (e.g., carbon, sulfur, or nitrogen); or both a change in the number of atoms and a replacement of the oxygen. Such structures may also comprise substitutions corresponding to those described for substituted sugar moieties (e.g., 6-membered carbocyclic bicyclic sugar surrogates optionally comprising additional substituents). Sugar surrogates also include more complex sugar replacements (e.g., the non-ring systems of peptide nucleic acid). Sugar surrogates include without limitation morpholino, modified morpholinos, cyclohexenyls and cyclohexitols.

As used herein, “nucleotide” means a nucleoside further comprising a phosphate linking group. As used herein, “linked nucleosides” may or may not be linked by phosphate linkages and thus includes, but is not limited to “linked nucleotides.” As used herein, “linked nucleosides” are nucleosides that are connected in a continuous sequence (i.e. no additional nucleosides are present between those that are linked).

As used herein, “nucleobase” means a group of atoms that can be linked to a sugar moiety to create a nucleoside that is capable of incorporation into an oligonucleotide, and wherein the group of atoms is capable of bonding with a complementary naturally occurring nucleobase of another oligonucleotide or nucleic acid. Nucleobases may be naturally occurring or may be modified.

As used herein, “heterocyclic base” or “heterocyclic nucleobase” means a nucleobase comprising a heterocyclic structure.

As used herein the terms, “unmodified nucleobase” or “naturally occurring nucleobase” means the naturally occurring heterocyclic nucleobases of RNA or DNA: the purine bases adenine (A) and guanine (G), and the pyrimidine bases thymine (T), cytosine (C) (including 5-methyl C), and uracil (U).

As used herein, “modified nucleobase” means any nucleobase that is not a naturally occurring nucleobase.

As used herein, “modified nucleoside” means a nucleoside comprising at least one chemical modification compared to naturally occurring RNA or DNA nucleosides. Modified nucleosides comprise a modified sugar moiety and/or a modified nucleobase.

As used herein, “bicyclic nucleoside” or “BNA” means a nucleoside comprising a bicyclic sugar moiety.

As used herein, “constrained ethyl nucleoside” or “cEt” means a nucleoside comprising a bicyclic sugar moiety comprising a 4′-CH(CH₃)—O-2′ bridge.

As used herein, “locked nucleic acid nucleoside” or “LNA” means a nucleoside comprising a bicyclic sugar moiety comprising a 4′-CH₂—O-2′ bridge.

As used herein, “2′-substituted nucleoside” means a nucleoside comprising a substituent at the 2′-position other than H or OH. Unless otherwise indicated, a 2′-substituted nucleoside is not a bicyclic nucleoside.

As used herein, “2′-deoxynucleoside” means a nucleoside comprising 2′-H furanosyl sugar moiety, as found in naturally occurring deoxyribonucleosides (DNA). In certain embodiments, a 2′-deoxynucleoside may comprise a modified nucleobase or may comprise an RNA nucleobase (e.g., uracil).

As used herein, “oligonucleotide” means a compound comprising a plurality of linked nucleosides. In certain embodiments, an oligonucleotide comprises one or more unmodified ribonucleosides (RNA) and/or unmodified deoxyribonucleosides (DNA) and/or one or more modified nucleosides.

As used herein “oligonucleoside” means an oligonucleotide in which none of the internucleoside linkages contains a phosphorus atom. As used herein, oligonucleotides include oligonucleosides.

As used herein, “modified oligonucleotide” means an oligonucleotide comprising at least one modified nucleoside and/or at least one modified internucleoside linkage.

As used herein “internucleoside linkage” means a covalent linkage between adjacent nucleosides in an oligonucleotide.

As used herein “naturally occurring internucleoside linkage” means a 3′ to 5′ phosphodiester linkage.

As used herein, “modified internucleoside linkage” means any internucleoside linkage other than a naturally occurring internucleoside linkage.

As used herein, “oligomeric compound” means a polymeric structure comprising two or more sub-structures. In certain embodiments, an oligomeric compound comprises an oligonucleotide. In certain embodiments, an oligomeric compound comprises one or more conjugate groups and/or terminal groups. In certain embodiments, an oligomeric compound consists of an oligonucleotide.

As used herein, “terminal group” means one or more atom attached to either, or both, the 3′ end or the 5′ end of an oligonucleotide. In certain embodiments a terminal group is a conjugate group. In certain embodiments, a terminal group comprises one or more terminal group nucleosides.

As used herein, “conjugate” means an atom or group of atoms bound to an oligonucleotide or oligomeric compound. In general, conjugate groups modify one or more properties of the compound to which they are attached, including, but not limited to pharmacodynamic, pharmacokinetic, binding, absorption, cellular distribution, cellular uptake, charge and/or clearance properties.

As used herein, “conjugate linking group” means any atom or group of atoms used to attach a conjugate to an oligonucleotide or oligomeric compound.

As used herein, “antisense compound” means a compound comprising or consisting of an oligonucleotide at least a portion of which is complementary to a target nucleic acid to which it is capable of hybridizing, resulting in at least one antisense activity.

As used herein, “antisense activity” means any detectable and/or measurable change attributable to the hybridization of an antisense compound to its target nucleic acid.

As used herein, “detecting” or “measuring” means that a test or assay for detecting or measuring is performed. Such detection and/or measuring may result in a value of zero. Thus, if a test for detection or measuring results in a finding of no activity (activity of zero), the step of detecting or measuring the activity has nevertheless been performed.

As used herein, “detectable and/or measurable activity” means a statistically significant activity that is not zero.

As used herein, “essentially unchanged” means little or no change in a particular parameter, particularly relative to another parameter which changes much more. In certain embodiments, a parameter is essentially unchanged when it changes less than 5%. In certain embodiments, a parameter is essentially unchanged if it changes less than two-fold while another parameter changes at least ten-fold. For example, in certain embodiments, an antisense activity is a change in the amount of a target nucleic acid. In certain such embodiments, the amount of a non-target nucleic acid is essentially unchanged if it changes much less than the target nucleic acid does, but the change need not be zero.

As used herein, “expression” means the process by which a gene ultimately results in a protein. Expression includes, but is not limited to, transcription, post-transcriptional modification (e.g., splicing, polyadenlyation, addition of 5′-cap), and translation.

As used herein, “target nucleic acid” means a nucleic acid molecule to which an antisense compound hybridizes.

As used herein, “mRNA” means an RNA molecule that encodes a protein.

As used herein, “pre-mRNA” means an RNA transcript that has not been fully processed into mRNA. Pre-RNA includes one or more intron.

As used herein, “transcript” means an RNA molecule transcribed from DNA. Transcripts include, but are not limited to mRNA, pre-mRNA, and partially processed RNA.

As used herein, “targeting” or “targeted to” means the association of an antisense compound to a particular target nucleic acid molecule or a particular region of a target nucleic acid molecule. An antisense compound targets a target nucleic acid if it is sufficiently complementary to the target nucleic acid to allow hybridization under physiological conditions.

As used herein, “nucleobase complementarity” or “complementarity” when in reference to nucleobases means a nucleobase that is capable of base pairing with another nucleobase. For example, in DNA, adenine (A) is complementary to thymine (T). For example, in RNA, adenine (A) is complementary to uracil (U). In certain embodiments, complementary nucleobase means a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair. Nucleobases comprising certain modifications may maintain the ability to pair with a counterpart nucleobase and thus, are still capable of nucleobase complementarity.

As used herein, “non-complementary” in reference to nucleobases means a pair of nucleobases that do not form hydrogen bonds with one another.

As used herein, “complementary” in reference to oligomeric compounds (e.g., linked nucleosides, oligonucleotides, or nucleic acids) means the capacity of such oligomeric compounds or regions thereof to hybridize to another oligomeric compound or region thereof through nucleobase complementarity under stringent conditions. Complementary oligomeric compounds need not have nucleobase complementarity at each nucleoside. Rather, some mismatches are tolerated. In certain embodiments, complementary oligomeric compounds or regions are complementary at 70% of the nucleobases (70% complementary). In certain embodiments, complementary oligomeric compounds or regions are 80% complementary. In certain embodiments, complementary oligomeric compounds or regions are 90% complementary. In certain embodiments, complementary oligomeric compounds or regions are 95% complementary. In certain embodiments, complementary oligomeric compounds or regions are 100% complementary.

As used herein, “hybridization” means the pairing of complementary oligomeric compounds (e.g., an antisense compound and its target nucleic acid). While not limited to a particular mechanism, the most common mechanism of pairing involves hydrogen bonding, which may be Watson-Crick, Hoogsteen or reversed Hoogsteen hydrogen bonding, between complementary nucleobases.

As used herein, “specifically hybridizes” means the ability of an oligomeric compound to hybridize to one nucleic acid site with greater affinity than it hybridizes to another nucleic acid site. In certain embodiments, an antisense oligonucleotide specifically hybridizes to more than one target site.

As used herein, “percent complementarity” means the percentage of nucleobases of an oligomeric compound that are complementary to an equal-length portion of a target nucleic acid. Percent complementarity is calculated by dividing the number of nucleobases of the oligomeric compound that are complementary to nucleobases at corresponding positions in the target nucleic acid by the total length of the oligomeric compound.

As used herein, “percent identity” means the number of nucleobases in a first nucleic acid that are the same type (independent of chemical modification) as nucleobases at corresponding positions in a second nucleic acid, divided by the total number of nucleobases in the first nucleic acid.

As used herein, “modulation” means a change of amount or quality of a molecule, function, or activity when compared to the amount or quality of a molecule, function, or activity prior to modulation. For example, modulation includes the change, either an increase (stimulation or induction) or a decrease (inhibition or reduction) in gene expression. As a further example, modulation of expression can include a change in splice site selection of pre-mRNA processing, resulting in a change in the absolute or relative amount of a particular splice-variant compared to the amount in the absence of modulation.

As used herein, “motif” means a pattern of chemical modifications in an oligomeric compound or a region thereof. Motifs may be defined by modifications at certain nucleosides and/or at certain linking groups of an oligomeric compound.

As used herein, “nucleoside motif” means a pattern of nucleoside modifications in an oligomeric compound or a region thereof. The linkages of such an oligomeric compound may be modified or unmodified. Unless otherwise indicated, motifs herein describing only nucleosides are intended to be nucleoside motifs. Thus, in such instances, the linkages are not limited.

As used herein, “sugar motif” means a pattern of sugar modifications in an oligomeric compound or a region thereof.

As used herein, “linkage motif” means a pattern of linkage modifications in an oligomeric compound or region thereof. The nucleosides of such an oligomeric compound may be modified or unmodified. Unless otherwise indicated, motifs herein describing only linkages are intended to be linkage motifs. Thus, in such instances, the nucleosides are not limited.

As used herein, “nucleobase modification motif” means a pattern of modifications to nucleobases along an oligonucleotide. Unless otherwise indicated, a nucleobase modification motif is independent of the nucleobase sequence.

As used herein, “sequence motif” means a pattern of nucleobases arranged along an oligonucleotide or portion thereof. Unless otherwise indicated, a sequence motif is independent of chemical modifications and thus may have any combination of chemical modifications, including no chemical modifications.

As used herein, “type of modification” in reference to a nucleoside or a nucleoside of a “type” means the chemical modification of a nucleoside and includes modified and unmodified nucleosides. Accordingly, unless otherwise indicated, a “nucleoside having a modification of a first type” may be an unmodified nucleoside.

As used herein, “differently modified” mean chemical modifications or chemical substituents that are different from one another, including absence of modifications. Thus, for example, a MOE nucleoside and an unmodified DNA nucleoside are “differently modified,” even though the DNA nucleoside is unmodified. Likewise, DNA and RNA are “differently modified,” even though both are naturally-occurring unmodified nucleosides. Nucleosides that are the same but for comprising different nucleobases are not differently modified. For example, a nucleoside comprising a 2′-OMe modified sugar and an unmodified adenine nucleobase and a nucleoside comprising a 2′-OMe modified sugar and an unmodified thymine nucleobase are not differently modified.

As used herein, “the same type of modifications” refers to modifications that are the same as one another, including absence of modifications. Thus, for example, two unmodified DNA nucleoside have “the same type of modification,” even though the DNA nucleoside is unmodified. Such nucleosides having the same type modification may comprise different nucleobases.

As used herein, “pharmaceutically acceptable carrier or diluent” means any substance suitable for use in administering to an animal. In certain embodiments, a pharmaceutically acceptable carrier or diluent is sterile saline. In certain embodiments, such sterile saline is pharmaceutical grade saline.

As used herein, “substituent” and “substituent group,” means an atom or group that replaces the atom or group of a named parent compound. For example a substituent of a modified nucleoside is any atom or group that differs from the atom or group found in a naturally occurring nucleoside (e.g., a modified 2′-substituent is any atom or group at the 2′-position of a nucleoside other than H or OH). Substituent groups can be protected or unprotected. In certain embodiments, compounds of the present invention have substituents at one or at more than one position of the parent compound. Substituents may also be further substituted with other substituent groups and may be attached directly or via a linking group such as an alkyl or hydrocarbyl group to a parent compound.

Likewise, as used herein, “substituent” in reference to a chemical functional group means an atom or group of atoms differs from the atom or a group of atoms normally present in the named functional group. In certain embodiments, a substituent replaces a hydrogen atom of the functional group (e.g., in certain embodiments, the substituent of a substituted methyl group is an atom or group other than hydrogen which replaces one of the hydrogen atoms of an unsubstituted methyl group). Unless otherwise indicated, groups amenable for use as substituents include without limitation, halogen, hydroxyl, alkyl, alkenyl, alkynyl, acyl (—C(O)Rₐₐ), carboxyl (—C(O)O—Rₐₐ), aliphatic groups, alicyclic groups, alkoxy, substituted oxy (—O—Rₐₐ), aryl, aralkyl, heterocyclic radical, heteroaryl, heteroarylalkyl, amino (—N(R_{bb})(R_{cc})), imino(═NR_{bb}), amido (—C(O)N(R_{bb})(R_{cc}) or —N(R_{bb})C(O)Rₐₐ), azido (—N₃), nitro (—NO₂), cyano (—CN), carbamido (—OC(O)N(R_{bb})(R_{cc}) or —N(R_{bb})C(O)ORₐₐ), ureido (—N(R_{bb})C(O)N(R_{bb})(R_{cc})), thioureido (—N(R_{bb})C(S)N(R_{bb})—(R_{cc})), guanidinyl (—N(R_{bb})C(═NR_{bb})N(R_{bb})(R_{cc})), amidinyl (—C(═NR_{bb})N(R_{bb})(R_{cc}) or —N(R_{bb})C(═NR_{bb})(Rₐₐ)), thiol (—SR_{bb}), sulfinyl (—S(O)R_{bb}), sulfonyl (—S(O)₂R_{bb}) and sulfonamidyl (—S(O)₂N(R_{bb})(R_{cc}) or —N(R_{bb})S—(O)₂R_{bb}). Wherein each Rₐₐ, R_{bb} and R_{cc} is, independently, H, an optionally linked chemical functional group or a further substituent group with a preferred list including without limitation, alkyl, alkenyl, alkynyl, aliphatic, alkoxy, acyl, aryl, aralkyl, heteroaryl, alicyclic, heterocyclic and heteroarylalkyl. Selected substituents within the compounds described herein are present to a recursive degree.

As used herein, “alkyl,” as used herein, means a saturated straight or branched hydrocarbon radical containing up to twenty four carbon atoms. Examples of alkyl groups include without limitation, methyl, ethyl, propyl, butyl, isopropyl, n-hexyl, octyl, decyl, dodecyl and the like. Alkyl groups typically include from 1 to about 24 carbon atoms, more typically from 1 to about 12 carbon atoms (C₁-C₁₂ alkyl) with from 1 to about 6 carbon atoms being more preferred.

As used herein, “alkenyl,” means a straight or branched hydrocarbon chain radical containing up to twenty four carbon atoms and having at least one carbon-carbon double bond. Examples of alkenyl groups include without limitation, ethenyl, propenyl, butenyl, 1-methyl-2-buten-1-yl, dienes such as 1,3-butadiene and the like. Alkenyl groups typically include from 2 to about 24 carbon atoms, more typically from 2 to about 12 carbon atoms with from 2 to about 6 carbon atoms being more preferred. Alkenyl groups as used herein may optionally include one or more further substituent groups.

As used herein, “alkynyl,” means a straight or branched hydrocarbon radical containing up to twenty four carbon atoms and having at least one carbon-carbon triple bond. Examples of alkynyl groups include, without limitation, ethynyl, 1-propynyl, 1-butynyl, and the like. Alkynyl groups typically include from 2 to about 24 carbon atoms, more typically from 2 to about 12 carbon atoms with from 2 to about 6 carbon atoms being more preferred. Alkynyl groups as used herein may optionally include one or more further substituent groups.

As used herein, “acyl,” means a radical formed by removal of a hydroxyl group from an organic acid and has the general Formula —C(O)—X where X is typically aliphatic, alicyclic or aromatic. Examples include aliphatic carbonyls, aromatic carbonyls, aliphatic sulfonyls, aromatic sulfinyls, aliphatic sulfinyls, aromatic phosphates, aliphatic phosphates and the like. Acyl groups as used herein may optionally include further substituent groups.

As used herein, “alicyclic” means a cyclic ring system wherein the ring is aliphatic. The ring system can comprise one or more rings wherein at least one ring is aliphatic. Preferred alicyclics include rings having from about 5 to about 9 carbon atoms in the ring. Alicyclic as used herein may optionally include further substituent groups.

As used herein, “aliphatic” means a straight or branched hydrocarbon radical containing up to twenty four carbon atoms wherein the saturation between any two carbon atoms is a single, double or triple bond.

An aliphatic group preferably contains from 1 to about 24 carbon atoms, more typically from 1 to about 12 carbon atoms with from 1 to about 6 carbon atoms being more preferred. The straight or branched chain of an aliphatic group may be interrupted with one or more heteroatoms that include nitrogen, oxygen, sulfur and phosphorus. Such aliphatic groups interrupted by heteroatoms include without limitation, polyalkoxys, such as polyalkylene glycols, polyamines, and polyimines Aliphatic groups as used herein may optionally include further substituent groups.

As used herein, “alkoxy” means a radical formed between an alkyl group and an oxygen atom wherein the oxygen atom is used to attach the alkoxy group to a parent molecule. Examples of alkoxy groups include without limitation, methoxy, ethoxy, propoxy, isopropoxy, n-butoxy, sec-butoxy, tert-butoxy, n-pentoxy, neopentoxy, n-hexoxy and the like. Alkoxy groups as used herein may optionally include further substituent groups.

As used herein, “aminoalkyl” means an amino substituted C₁-C₁₂ alkyl radical. The alkyl portion of the radical forms a covalent bond with a parent molecule. The amino group can be located at any position and the aminoalkyl group can be substituted with a further substituent group at the alkyl and/or amino portions.

As used herein, “aralkyl” and “arylalkyl” mean an aromatic group that is covalently linked to a C₁-C₁₂ alkyl radical. The alkyl radical portion of the resulting aralkyl (or arylalkyl) group forms a covalent bond with a parent molecule. Examples include without limitation, benzyl, phenethyl and the like. Aralkyl groups as used herein may optionally include further substituent groups attached to the alkyl, the aryl or both groups that form the radical group.

As used herein, “aryl” and mean a mono- or polycyclic carbocyclic ring system radicals having one or more aromatic rings. Examples of aryl groups include without limitation, phenyl, naphthyl, tetrahydronaphthyl, indanyl, idenyl and the like. Preferred aryl ring systems have from about 5 to about 20 carbon atoms in one or more rings. Aryl groups as used herein may optionally include further substituent groups.

As used herein, “halo” and “halogen,” mean an atom selected from fluorine, chlorine, bromine and iodine.

As used herein, “heteroaryl,” mean a radical comprising a mono- or poly-cyclic aromatic ring, ring system or fused ring system wherein at least one of the rings is aromatic and includes one or more heteroatoms. Heteroaryl is also meant to include fused ring systems including systems where one or more of the fused rings contain no heteroatoms. Heteroaryl groups typically include one ring atom selected from sulfur, nitrogen or oxygen. Examples of heteroaryl groups include without limitation, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl and the like. Heteroaryl radicals can be attached to a parent molecule directly or through a linking moiety such as an aliphatic group or hetero atom. Heteroaryl groups as used herein may optionally include further substituent groups.

### Oligomeric Compounds

In certain embodiments, the present invention provides oligomeric compounds. In certain embodiments, such oligomeric compounds comprise oligonucleotides optionally comprising one or more conjugate and/or terminal groups. In certain embodiments, an oligomeric compound consists of an oligonucleotide. In certain embodiments, oligonucleotides comprise one or more chemical modifications. Such chemical modifications include modifications one or more nucleoside (including modifications to the sugar moiety and/or the nucleobase) and/or modifications to one or more internucleoside linkage.

Certain Sugar Moieties

In certain embodiments, oligomeric compounds of the invention comprise one or more modified nucleosides comprising a modified sugar moiety. Such oligomeric compounds comprising one or more sugar-modified nucleosides may have desirable properties, such as enhanced nuclease stability or increased binding affinity with a target nucleic acid relative to oligomeric compounds comprising only nucleosides comprising naturally occurring sugar moieties. In certain embodiments, modified sugar moieties are substituted sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of substituted sugar moieties.

In certain embodiments, modified sugar moieties are substituted sugar moieties comprising one or more substituent, including but not limited to substituents at the 2′ and/or 5′ positions. Examples of sugar substituents suitable for the 2′-position, include, but are not limited to: 2′-F, 2′-OCH₃ (“OMe” or “O-methyl”), and 2′-O(CH₂)₂OCH₃ (“MOE”). In certain embodiments, sugar substituents at the 2′ position is selected from allyl, amino, azido, thio, O-allyl, O—C₁-C₁₀ alkyl, O—C₁-C₁₀ substituted alkyl; O—C₁-C₁₀ alkoxy; O—C₁-C₁₀ substituted alkoxy, OCF₃, O(CH₂)₂SCH₃, O(CH₂)₂—O—N(Rm)(Rn), and O—CH₂—C(═O)—N(Rm)(Rn), where each Rm and Rn is, independently, H or substituted or unsubstituted C₁-C₁₀ alkyl. Examples of sugar substituents at the 5′-position, include, but are not limited to: 5′-methyl (R or S); 5′-vinyl, and 5′-methoxy. In certain embodiments, substituted sugars comprise more than one non-bridging sugar substituent, for example, 2′-F-5′-methyl sugar moieties (see, e.g., PCT International Application WO 2008/101157, for additional 5′,2′-bis substituted sugar moieties and nucleosides).

Nucleosides comprising 2′-substituted sugar moieties are referred to as 2′-substituted nucleosides. In certain embodiments, a 2′-substituted nucleoside comprises a 2′-substituent group selected from halo, allyl, amino, azido, O—C₁-C₁₀ alkoxy; O—C₁-C₁₀ substituted alkoxy, SH, CN, OCN, CF₃, OCF₃, O-alkyl, S-alkyl, N(Rₘ)-alkyl; O-alkenyl, S-alkenyl, or N(Rₘ)-alkenyl; O-alkynyl, S-alkynyl, N(Rₘ)-alkynyl; O-alkylenyl-O-alkyl, alkynyl, alkaryl, aralkyl, O-alkaryl, O-aralkyl, O(CH₂)₂SCH₃, O—(CH₂)₂—O—N(Rₘ)(Rₙ) or O—CH₂—C(═O)—N(Rₘ)(Rₙ), where each Rₘ and Rₙ is, independently, H, an amino protecting group or substituted or unsubstituted C₁-C₁₀ alkyl. These 2′-substituent groups can be further substituted with one or more substituent groups independently selected from hydroxyl, amino, alkoxy, carboxy, benzyl, phenyl, nitro (NO₂), thiol, thioalkoxy (S-alkyl), halogen, alkyl, aryl, alkenyl and alkynyl.

In certain embodiments, a 2′-substituted nucleoside comprises a 2′-substituent group selected from F, NH₂, N₃, OCF₃, O—CH₃, O(CH₂)₃NH₂, CH₂—CH═CH₂, O—CH₂—CH═CH₂, OCH₂CH₂OCH₃, O(CH₂)₂SCH₃, O—(CH₂)₂—O—N(Rₘ)(Rₙ), O(CH₂)₂O(CH₂)₂N(CH₃)₂, and N-substituted acetamide (O—CH₂—C(═O)—N(Rₘ)(Rₙ) where each Rₘ and Rₙ is, independently, H, an amino protecting group or substituted or unsubstituted C₁-C₁₀ alkyl.

In certain embodiments, a 2′-substituted nucleoside comprises a sugar moiety comprising a 2′-substituent group selected from F, OCF₃, O—CH₃, OCH₂CH₂OCH₃, O(CH₂)₂SCH₃, O—(CH₂)₂—O—N(CH₃)₂, —O(CH₂)₂O(CH₂)₂N(CH₃)₂, and O—CH₂—C(═O)—N(H)CH₃.

In certain embodiments, a 2′-substituted nucleoside comprises a sugar moiety comprising a 2′-substituent group selected from F, O—CH₃, and OCH₂CH₂OCH₃.

Certain modified sugar moieties comprise a bridging sugar substituent that forms a second ring resulting in a bicyclic sugar moiety. In certain such embodiments, the bicyclic sugar moiety comprises a bridge between the 4′ and the 2′ furanose ring atoms. Examples of such 4′ to 2′ sugar substituents, include, but are not limited to: —[C(Rₐ)(R_{b})]ₙ—, —[C(Rₐ)(R_{b})]ₙ—O—, —C(RₐR_{b})—N(R)—O— or, —C(RₐR_{b})—O—N(R)—; 4′-CH₂-2′, 4′-(CH₂)₂-2′, 4′-(CH₂)₃-2′, 4′-(CH₂)—O-2′ (LNA); 4′-(CH₂)—S-2; 4′-(CH₂)₂—O-2′ (ENA); 4′-CH(CH₃)—O-2′ (cEt) and 4′-CH(CH₂OCH₃)—O-2′, and analogs thereof (see, e.g., U.S. Pat. No. 7,399,845, issued on Jul. 15, 2008); 4′-C(CH₃)(CH₃)—O-2′ and analogs thereof, (see, e.g., WO2009/006478, published Jan. 8, 2009); 4′-CH₂—N(OCH₃)-2′ and analogs thereof (see, e.g., WO2008/150729, published Dec. 11, 2008); 4′-CH₂—O—N(CH₃)-2′ (see, e.g., US2004/0171570, published Sep. 2, 2004); 4′-CH₂—O—N(R)-2′, and 4′-CH₂—N(R)—O-2′-, wherein each R is, independently, H, a protecting group, or C₁-C₁₂ alkyl; 4′-CH₂—N(R)—O-2′, wherein R is H, C₁-C₁₂ alkyl, or a protecting group (see, U.S. Pat. No. 7,427,672, issued on Sep. 23, 2008); 4′-CH₂—C(H)(CH₃)-2′ (see, e.g., Chattopadhyaya, et al., J. Org. Chem., 2009, 74, 118-134); and 4′-CH₂—C(═CH₂)-2′ and analogs thereof (see, published PCT International Application WO 2008/154401, published on Dec. 8, 2008).

In certain embodiments, such 4′ to 2′ bridges independently comprise from 1 to 4 linked groups independently selected from —[C(Rₐ)(R_{b})]ₐ—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—;

wherein:

x is 0, 1, or 2;

n is 1, 2, 3, or 4;

each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl(C(═O)—H), substituted acyl, CN, sulfonyl (S(═O)₂-J₁), or sulfoxyl (S(═O)-J₁); and

each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Nucleosides comprising bicyclic sugar moieties are referred to as bicyclic nucleosides or BNAs. Bicyclic nucleosides include, but are not limited to, (A) α-L-Methyleneoxy(4′-CH₂—O-2′) BNA, (B) β-D-Methyleneoxy (4′-CH₂—O-2′) BNA (also referred to as locked nucleic acid or LNA), (C) Ethyleneoxy (4′-(CH₂)₂—O-2′) BNA, (D) Aminooxy (4′-CH₂—O—N(R)-2′) BNA, (E) Oxyamino (4′-CH₂—N(R)—O-2′) BNA, (F) Methyl(methyleneoxy) (4′-CH(CH₃)—O-2′) BNA (also referred to as constrained ethyl or cEt), (G) methylene-thio (4′-CH₂—S-2′) BNA, (H) methylene-amino (4′-CH₂—N(R)-2′) BNA, (I) methyl carbocyclic (4′-CH₂—CH(CH₃)-2′) BNA, and (J) propylene carbocyclic (4′-(CH₂)₃-2′) BNA as depicted below.

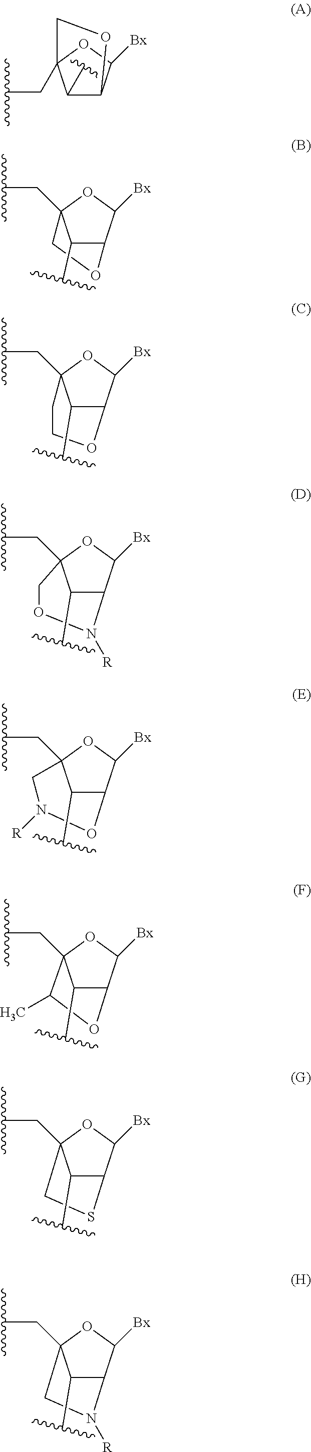

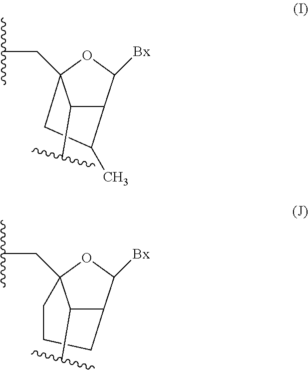

wherein Bx is a nucleobase moiety and R is, independently, H, a protecting group, or C₁-C₁₂ alkyl.

Additional bicyclic sugar moieties are known in the art, for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A., 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 129 (26) 8362-8379 (Jul. 4, 2007); Elayadi et al., Curr. Opinion Invens. Drugs, 2001, 2, 558-561; Braasch et al., Chem. Biol., 2001, 8, 1-7; Orum et al., Curr. Opinion Mol. Ther., 2001, 3, 239-243; U.S. Pat. Nos. 7,053,207, 6,268,490, 6,770,748, 6,794,499, 7,034,133, 6,525,191, 6,670,461, and 7,399,845; WO 2004/106356, WO 1994/14226, WO 2005/021570, and WO 2007/134181; U.S. Patent Publication Nos. US2004/0171570, US2007/0287831, and US2008/0039618; U.S. patent Ser. Nos. 12/129,154, 60/989,574, 61/026,995, 61/026,998, 61/056,564, 61/086,231, 61/097,787, and 61/099,844; and PCT International Applications Nos. PCT/US2008/064591, PCT/US2008/066154, and PCT/US2008/068922.

In certain embodiments, bicyclic sugar moieties and nucleosides incorporating such bicyclic sugar moieties are further defined by isomeric configuration. For example, a nucleoside comprising a 4′-2′ methylene-oxy bridge, may be in the α-L configuration or in the β-D configuration. Previously, α-L-methyleneoxy (4′-CH₂—O-2′) bicyclic nucleosides have been incorporated into antisense oligonucleotides that showed antisense activity (Frieden et al., Nucleic Acids Research, 2003, 21, 6365-6372).

In certain embodiments, substituted sugar moieties comprise one or more non-bridging sugar substituent and one or more bridging sugar substituent (e.g., 5′-substituted and 4′-2′ bridged sugars). (see, PCT International Application WO 2007/134181, published on Nov. 22, 2007, wherein LNA is substituted with, for example, a 5′-methyl or a 5′-vinyl group).

In certain embodiments, modified sugar moieties are sugar surrogates. In certain such embodiments, the oxygen atom of the naturally occurring sugar is substituted, e.g., with a sulfur, carbon or nitrogen atom. In certain such embodiments, such modified sugar moiety also comprises bridging and/or non-bridging substituents as described above. For example, certain sugar surogates comprise a 4′-sulfur atom and a substitution at the 2′-position (see, e.g., published U.S. Patent Application US2005/0130923, published on Jun. 16, 2005) and/or the 5′ position. By way of additional example, carbocyclic bicyclic nucleosides having a 4′-2′ bridge have been described (see, e.g., Freier et al., Nucleic Acids Research, 1997, 25(22), 4429-4443 and Albaek et al., J. Org. Chem., 2006, 71, 7731-7740).

In certain embodiments, sugar surrogates comprise rings having other than 5-atoms. For example, in certain embodiments, a sugar surrogate comprises a six-membered tetrahydropyran. Such tetrahydropyrans may be further modified or substituted. Nucleosides comprising such modified tetrahydropyrans include, but are not limited to, hexitol nucleic acid (HNA), anitol nucleic acid (ANA), manitol nucleic acid (MNA) (see Leumann, C J. Bioorg. & Med. Chem. (2002) 10:841-854), fluoro HNA (F-HNA), and those compounds having Formula VII:

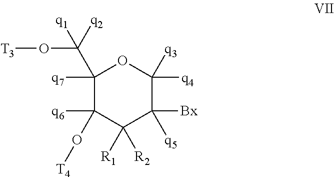

wherein independently for each of said at least one tetrahydropyran nucleoside analog of Formula VII:

Bx is a nucleobase moiety;

T₃ and T₄ are each, independently, an internucleoside linking group linking the tetrahydropyran nucleoside analog to the antisense compound or one of T₃ and T₄ is an internucleoside linking group linking the tetrahydropyran nucleoside analog to the antisense compound and the other of T₃ and T₄ is H, a hydroxyl protecting group, a linked conjugate group, or a 5′ or 3′-terminal group;

q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and

each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, the modified THP nucleosides of Formula VII are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, THP nucleosides of Formula VII are provided wherein one of R₁ and R₂ is F. In certain embodiments, R₁ is fluoro and R₂ is H, R₁ is methoxy and R₂ is H, and R₁ is methoxyethoxy and R₂ is H.

Many other bicyclic and tricyclic sugar and sugar surrogate ring systems are known in the art that can be used to modify nucleosides (see, e.g., review article: Leumann, J. C, Bioorganic & Medicinal Chemistry, 2002, 10, 841-854).

In certain embodiments, sugar surrogates comprise rings having more than 5 atoms and more than one heteroatom. For example nucleosides comprising morpholino sugar moieties and their use in oligomeric compounds has been reported (see for example: Braasch et al., Biochemistry, 2002, 41, 4503-4510; and U.S. Pat. Nos. 5,698,685; 5,166,315; 5,185,444; and 5,034,506). As used here, the term “morpholino” means a sugar surrogate having the following structure:

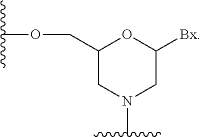

In certain embodiments, morpholinos may be modified, for example by adding or altering various substituent groups from the above morpholino structure. Such sugar surrogates are referred to herein as “modified morpholinos.”

Combinations of modifications are also provided without limitation, such as 2′-F-5′-methyl substituted nucleosides (see PCT International Application WO 2008/101157 Published on Aug. 21, 2008 for other disclosed 5′,2′-bis substituted nucleosides) and replacement of the ribosyl ring oxygen atom with S and further substitution at the 2′-position (see published U.S. Patent Application US2005-0130923, published on Jun. 16, 2005) or alternatively 5′-substitution of a bicyclic nucleic acid (see PCT International Application WO 2007/134181, published on Nov. 22, 2007 wherein a 4′-CH₂—O-2′ bicyclic nucleoside is further substituted at the 5′ position with a 5′-methyl or a 5′-vinyl group). The synthesis and preparation of carbocyclic bicyclic nucleosides along with their oligomerization and biochemical studies have also been described (see, e.g., Srivastava et al., J. Am. Chem. Soc. 2007, 129(26), 8362-8379).

Certain Nucleobases

In certain embodiments, nucleosides of the present invention comprise one or more unmodified nucleobases. In certain embodiments, nucleosides of the present invention comprise one or more modified nucleobases.

In certain embodiments, modified nucleobases are selected from: universal bases, hydrophobic bases, promiscuous bases, size-expanded bases, and fluorinated bases as defined herein. 5-substituted pyrimidines, 6-azapyrimidines and N-2, N-6 and 0-6 substituted purines, including 2-aminopropyladenine, 5-propynyluracil; 5-propynylcytosine; 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, 3-deazaguanine and 3-deazaadenine, universal bases, hydrophobic bases, promiscuous bases, size-expanded bases, and fluorinated bases as defined herein. Further modified nucleobases include tricyclic pyrimidines such as phenoxazine cytidine([5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, Kroschwitz, J. I., Ed., John Wiley & Sons, 1990, 858-859; those disclosed by Englisch et al., Angewandte Chemie, International Edition, 1991, 30, 613; and those disclosed by Sanghvi, Y. S., Chapter 15, Antisense Research and Applications, Crooke, S. T. and Lebleu, B., Eds., CRC Press, 1993, 273-288.

Representative United States patents that teach the preparation of certain of the above noted modified nucleobases as well as other modified nucleobases include without limitation, U.S. Pat. Nos. 3,687,808; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; 5,645,985; 5,681,941; 5,750,692; 5,763,588; 5,830,653 and 6,005,096, certain of which are commonly owned with the instant application, and each of which is herein incorporated by reference in its entirety.

Certain Internucleoside Linkages

In certain embodiments, the present invention provides oligomeric compounds comprising linked nucleosides. In such embodiments, nucleosides may be linked together using any internucleoside linkage. The two main classes of internucleoside linking groups are defined by the presence or absence of a phosphorus atom. Representative phosphorus containing internucleoside linkages include, but are not limited to, phosphodiesters (P═O), phosphotriesters, methylphosphonates, phosphoramidate, and phosphorothioates (P═S). Representative non-phosphorus containing internucleoside linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). Modified linkages, compared to natural phosphodiester linkages, can be used to alter, typically increase, nuclease resistance of the oligomeric compound. In certain embodiments, internucleoside linkages having a chiral atom can be prepared as a racemic mixture, or as separate enantiomers. Representative chiral linkages include, but are not limited to, alkylphosphonates and phosphorothioates. Methods of preparation of phosphorous-containing and non-phosphorous-containing internucleoside linkages are well known to those skilled in the art.

The oligonucleotides described herein contain one or more asymmetric centers and thus give rise to enantiomers, diastereomers, and other stereoisomeric configurations that may be defined, in terms of absolute stereochemistry, as (R) or (S), α or β such as for sugar anomers, or as (D) or (L) such as for amino acids etc. Included in the antisense compounds provided herein are all such possible isomers, as well as their racemic and optically pure forms.

Neutral internucleoside linkages include without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

Certain Motifs

In certain embodiments, the present invention provides oligomeric compounds comprising oligonucleotides. In certain embodiments, such oligonucleotides comprise one or more chemical modification. In certain embodiments, chemically modified oligonucleotides comprise one or more modified nucleosides. In certain embodiments, chemically modified oligonucleotides comprise one or more modified nucleosides comprising modified sugars. In certain embodiments, chemically modified oligonucleotides comprise one or more modified nucleosides comprising one or more modified nucleobases. In certain embodiments, chemically modified oligonucleotides comprise one or more modified internucleoside linkages. In certain embodiments, the chemically modifications (sugar modifications, nucleobase modifications, and/or linkage modifications) define a pattern or motif. In certain embodiments, the patterns of chemical modifications of sugar moieties, internucleoside linkages, and nucleobases are each independent of one another. Thus, an oligonucleotide may be described by its sugar modification motif, internucleoside linkage motif and/or nucleobase modification motif (as used herein, nucleobase modification motif describes the chemical modifications to the nucleobases independent of the sequence of nucleobases).

Certain Sugar Motifs

In certain embodiments, oligonucleotides comprise one or more type of modified sugar moieties and/or naturally occurring sugar moieties arranged along an oligonucleotide or region thereof in a defined pattern or sugar modification motif. Such motifs may include any of the sugar modifications discussed herein and/or other known sugar modifications.

In certain embodiments, the oligonucleotides comprise or consist of a region having a gapmer sugar modification motif, which comprises two external regions or “wings” and an internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap. In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar modification motifs of the two wings are the same as one another (symmetric gapmer). In certain embodiments, the sugar modification motifs of the 5′-wing differs from the sugar modification motif of the 3′-wing (asymmetric gapmer). In certain embodiments, oligonucleotides comprise 2′-MOE modified nucleosides in the wings and 2′-F modified nucleosides in the gap.

In certain embodiments, oligonucleotides are fully modified. In certain such embodiments, oligonucleotides are uniformly modified. In certain embodiments, oligonucleotides are uniform 2′-MOE. In certain embodiments, oligonucleotides are uniform 2′-F. In certain embodiments, oligonucleotides are uniform morpholino. In certain embodiments, oligonucleotides are uniform BNA. In certain embodiments, oligonucleotides are uniform LNA. In certain embodiments, oligonucleotides are uniform cEt.

In certain embodiments, oligonucleotides comprise a uniformly modified region and additional nucleosides that are unmodified or differently modified. In certain embodiments, the uniformly modified region is at least 5, 10, 15, or 20 nucleosides in length. In certain embodiments, the uniform region is a 2′-MOE region. In certain embodiments, the uniform region is a 2′-F region. In certain embodiments, the uniform region is a morpholino region. In certain embodiments, the uniform region is a BNA region. In certain embodiments, the uniform region is a LNA region. In certain embodiments, the uniform region is a cEt region.

In certain embodiments, the oligonucleotide does not comprise more than 4 contiguous unmodified 2′-deoxynucleosides. In certain circumstances, antisesense oligonucleotides comprising more than 4 contiguous 2′-deoxynucleosides activate RNase H, resulting in cleavage of the target RNA. In certain embodiments, such cleavage is avoided by not having more than 4 contiguous 2′-deoxynucleosides, for example, where alteration of splicing and not cleavage of a target RNA is desired.

Certain Internucleoside Linkage Motifs

In certain embodiments, oligonucleotides comprise modified internucleoside linkages arranged along the oligonucleotide or region thereof in a defined pattern or modified internucleoside linkage motif. In certain embodiments, internucleoside linkages are arranged in a gapped motif, as described above for sugar modification motif. In such embodiments, the internucleoside linkages in each of two wing regions are different from the internucleoside linkages in the gap region. In certain embodiments the internucleoside linkages in the wings are phosphodiester and the internucleoside linkages in the gap are phosphorothioate. The sugar modification motif is independently selected, so such oligonucleotides having a gapped internucleoside linkage motif may or may not have a gapped sugar modification motif and if it does have a gapped sugar motif, the wing and gap lengths may or may not be the same.

In certain embodiments, oligonucleotides comprise a region having an alternating internucleoside linkage motif. In certain embodiments, oligonucleotides of the present invention comprise a region of uniformly modified internucleoside linkages. In certain such embodiments, the oligonucleotide comprises a region that is uniformly linked by phosphorothioate internucleoside linkages. In certain embodiments, the oligonucleotide is uniformly linked by phosphorothioate. In certain embodiments, each internucleoside linkage of the oligonucleotide is selected from phosphodiester and phosphorothioate. In certain embodiments, each internucleoside linkage of the oligonucleotide is selected from phosphodiester and phosphorothioate and at least one internucleoside linkage is phosphorothioate.

In certain embodiments, the oligonucleotide comprises at least 6 phosphorothioate internucleoside linkages. In certain embodiments, the oligonucleotide comprises at least 8 phosphorothioate internucleoside linkages. In certain embodiments, the oligonucleotide comprises at least 10 phosphorothioate internucleoside linkages. In certain embodiments, the oligonucleotide comprises at least one block of at least 6 consecutive phosphorothioate internucleoside linkages. In certain embodiments, the oligonucleotide comprises at least one block of at least 8 consecutive phosphorothioate internucleoside linkages. In certain embodiments, the oligonucleotide comprises at least one block of at least 10 consecutive phosphorothioate internucleoside linkages. In certain embodiments, the oligonucleotide comprises at least block of at least one 12 consecutive phosphorothioate internucleoside linkages. In certain such embodiments, at least one such block is located at the 3′ end of the oligonucleotide. In certain such embodiments, at least one such block is located within 3 nucleosides of the 3′ end of the oligonucleotide.

Certain Nucleobase Modification Motifs

In certain embodiments, oligonucleotides comprise chemical modifications to nucleobases arranged along the oligonucleotide or region thereof in a defined pattern or nucleobases modification motif. In certain such embodiments, nucleobase modifications are arranged in a gapped motif. In certain embodiments, nucleobase modifications are arranged in an alternating motif. In certain embodiments, each nucleobase is modified. In certain embodiments, none of the nucleobases is chemically modified.

In certain embodiments, oligonucleotides comprise a block of modified nucleobases. In certain such embodiments, the block is at the 3′-end of the oligonucleotide. In certain embodiments the block is within 3 nucleotides of the 3′-end of the oligonucleotide. In certain such embodiments, the block is at the 5′-end of the oligonucleotide. In certain embodiments the block is within 3 nucleotides of the 5′-end of the oligonucleotide.

In certain embodiments, nucleobase modifications are a function of the natural base at a particular position of an oligonucleotide. For example, in certain embodiments each purine or each pyrimidine in an oligonucleotide is modified. In certain embodiments, each adenine is modified. In certain embodiments, each guanine is modified. In certain embodiments, each thymine is modified. In certain embodiments, each cytosine is modified. In certain embodiments, each uracil is modified.

In certain embodiments, some, all, or none of the cytosine moieties in an oligonucleotide are 5-methyl cytosine moieties. Herein, 5-methyl cytosine is not a “modified nucleobase.” Accordingly, unless otherwise indicated, unmodified nucleobases include both cytosine residues having a 5-methyl and those lacking a 5 methyl. In certain embodiments, the methylation state of all or some cytosine nucleobases is specified.

Certain Overall Lengths

In certain embodiments, the present invention provides oligomeric compounds including oligonucleotides of any of a variety of ranges of lengths. In certain embodiments, the invention provides oligomeric compounds or oligonucleotides consisting of X to Y linked nucleosides, where X represents the fewest number of nucleosides in the range and Y represents the largest number of nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≦Y. For example, in certain embodiments, the invention provides oligomeric compounds which comprise oligonucleotides consisting of 8 to 9, 8 to 10, 8 to 11, 8 to 12, 8 to 13, 8 to 14, 8 to 15, 8 to 16, 8 to 17, 8 to 18, 8 to 19, 8 to 20, 8 to 21, 8 to 22, 8 to 23, 8 to 24, 8 to 25, 8 to 26, 8 to 27, 8 to 28, 8 to 29, 8 to 30, 9 to 10, 9 to 11, 9 to 12, 9 to 13, 9 to 14, 9 to 15, 9 to 16, 9 to 17, 9 to 18, 9 to 19, 9 to 20, 9 to 21, 9 to 22, 9 to 23, 9 to 24, 9 to 25, 9 to 26, 9 to 27, 9 to 28, 9 to 29, 9 to 30, 10 to 11, 10 to 12, 10 to 13, 10 to 14, 10 to 15, 10 to 16, 10 to 17, 10 to 18, 10 to 19, 10 to 20, 10 to 21, 10 to 22, 10 to 23, 10 to 24, 10 to 25, 10 to 26, 10 to 27, 10 to 28, 10 to 29, 10 to 30, 11 to 12, 11 to 13, 11 to 14, 11 to 15, 11 to 16, 11 to 17, 11 to 18, 11 to 19, 11 to 20, 11 to 21, 11 to 22, 11 to 23, 11 to 24, 11 to 25, 11 to 26, 11 to 27, 11 to 28, 11 to 29, 11 to 30, 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides. In embodiments where the number of nucleosides of an oligomeric compound or oligonucleotide is limited, whether to a range or to a specific number, the oligomeric compound or oligonucleotide may, nonetheless further comprise additional other substituents. For example, an oligonucleotide comprising 8-30 nucleosides excludes oligonucleotides having 31 nucleosides, but, unless otherwise indicated, such an oligonucleotide may further comprise, for example one or more conjugates, terminal groups, or other substituents. In certain embodiments, a gapmer oligonucleotide has any of the above lengths.

One of skill in the art will appreciate that certain lengths may not be possible for certain motifs. For example: a gapmer having a 5′-wing region consisting of four nucleotides, a gap consisting of at least six nucleotides, and a 3′-wing region consisting of three nucleotides cannot have an overall length less than 13 nucleotides. Thus, one would understand that the lower length limit is 13 and that the limit of 10 in “10-20” has no effect in that embodiment.

Further, where an oligonucleotide is described by an overall length range and by regions having specified lengths, and where the sum of specified lengths of the regions is less than the upper limit of the overall length range, the oligonucleotide may have additional nucleosides, beyond those of the specified regions, provided that the total number of nucleosides does not exceed the upper limit of the overall length range. For example, an oligonucleotide consisting of 20-25 linked nucleosides comprising a 5′-wing consisting of 5 linked nucleosides; a 3′-wing consisting of 5 linked nucleosides and a central gap consisting of 10 linked nucleosides (5+5+10=20) may have up to 5 nucleosides that are not part of the 5′-wing, the 3′-wing, or the gap (before reaching the overall length limitation of 25). Such additional nucleosides may be 5′ of the 5′-wing and/or 3′ of the 3′ wing.

Certain Oligonucleotides

In certain embodiments, oligonucleotides of the present invention are characterized by their sugar motif, internucleoside linkage motif, nucleobase modification motif and overall length. In certain embodiments, such parameters are each independent of one another. Thus, each internucleoside linkage of an oligonucleotide having a gapmer sugar motif may be modified or unmodified and may or may not follow the gapmer modification pattern of the sugar modifications. Thus, the internucleoside linkages within the wing regions of a sugar-gapmer may be the same or different from one another and may be the same or different from the internucleoside linkages of the gap region. Likewise, such sugar-gapmer oligonucleotides may comprise one or more modified nucleobase independent of the gapmer pattern of the sugar modifications. Herein if a description of an oligonucleotide or oligomeric compound is silent with respect to one or more parameter, such parameter is not limited. Thus, an oligomeric compound described only as having a gapmer sugar motif without further description may have any length, internucleoside linkage motif, and nucleobase modification motif. Unless otherwise indicated, all chemical modifications are independent of nucleobase sequence.

Certain Conjugate Groups

In certain embodiments, oligomeric compounds are modified by attachment of one or more conjugate groups. In general, conjugate groups modify one or more properties of the attached oligomeric compound including but not limited to pharmacodynamics, pharmacokinetics, stability, binding, absorption, cellular distribution, cellular uptake, charge and clearance. Conjugate groups are routinely used in the chemical arts and are linked directly or via an optional conjugate linking moiety or conjugate linking group to a parent compound such as an oligomeric compound, such as an oligonucleotide. Conjugate groups includes without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes. Certain conjugate groups have been described previously, for example: cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937).

In certain embodiments, a conjugate group comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

In certain embodiments, conjugate groups are directly attached to oligonucleotides in oligomeric compounds. In certain embodiments, conjugate groups are attached to oligonucleotides by a conjugate linking group. In certain such embodiments, conjugate linking groups, including, but not limited to, bifunctional linking moieties such as those known in the art are amenable to the compounds provided herein. Conjugate linking groups are useful for attachment of conjugate groups, such as chemical stabilizing groups, functional groups, reporter groups and other groups to selective sites in a parent compound such as for example an oligomeric compound. In general a bifunctional linking moiety comprises a hydrocarbyl moiety having two functional groups. One of the functional groups is selected to bind to a parent molecule or compound of interest and the other is selected to bind essentially any selected group such as chemical functional group or a conjugate group. In some embodiments, the conjugate linker comprises a chain structure or an oligomer of repeating units such as ethylene glycol or amino acid units. Examples of functional groups that are routinely used in a bifunctional linking moiety include, but are not limited to, electrophiles for reacting with nucleophilic groups and nucleophiles for reacting with electrophilic groups. In some embodiments, bifunctional linking moieties include amino, hydroxyl, carboxylic acid, thiol, unsaturations (e.g., double or triple bonds), and the like.

Some nonlimiting examples of conjugate linking moieties include pyrrolidine, 8-amino-3,6-dioxaoctanoic acid (ADO), succinimidyl 4-(N-maleimidomethyl)cyclohexane-1-carboxylate (SMCC) and 6-aminohexanoic acid (AHEX or AHA). Other linking groups include, but are not limited to, substituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

Conjugate groups may be attached to either or both ends of an oligonucleotide (terminal conjugate groups) and/or at any internal position.

In certain embodiments, conjugate groups are at the 3′-end of an oligonucleotide of an oligomeric compound. In certain embodiments, conjugate groups are near the 3′-end. In certain embodiments, conjugates are attached at the 3′ end of an oligomeric compound, but before one or more terminal group nucleosides. In certain embodiments, conjugate groups are placed within a terminal group.

In certain embodiments, the present invention provides oligomeric compounds. In certain embodiments, oligomeric compounds comprise an oligonucleotide. In certain embodiments, an oligomeric compound comprises an oligonucleotide and one or more conjugate and/or terminal groups. Such conjugate and/or terminal groups may be added to oligonucleotides having any of the chemical motifs discussed above. Thus, for example, an oligomeric compound comprising an oligonucleotide having region of alternating nucleosides may comprise a terminal group.

### Antisense Compounds

In certain embodiments, oligomeric compounds of the present invention are antisense compounds. Such antisense compounds are capable of hybridizing to a target nucleic acid, resulting in at least one antisense activity. In certain embodiments, antisense compounds specifically hybridize to one or more target nucleic acid. In certain embodiments, a specifically hybridizing antisense compound has a nucleobase sequence comprising a region having sufficient complementarity to a target nucleic acid to allow hybridization and result in antisense activity and insufficient complementarity to any non-target so as to avoid non-specific hybridization to any non-target nucleic acid sequences under conditions in which specific hybridization is desired (e.g., under physiological conditions for in vivo or therapeutic uses, and under conditions in which assays are performed in the case of in vitro assays).

In certain embodiments, the present invention provides antisense compounds comprising oligonucleotides that are fully complementary to the target nucleic acid over the entire length of the oligonucleotide. In certain embodiments, oligonucleotides are 99% complementary to the target nucleic acid. In certain embodiments, oligonucleotides are 95% complementary to the target nucleic acid. In certain embodiments, such oligonucleotides are 90% complementary to the target nucleic acid.

In certain embodiments, such oligonucleotides are 85% complementary to the target nucleic acid. In certain embodiments, such oligonucleotides are 80% complementary to the target nucleic acid. In certain embodiments, an antisense compound comprises a region that is fully complementary to a target nucleic acid and is at least 80% complementary to the target nucleic acid over the entire length of the oligonucleotide. In certain such embodiments, the region of full complementarity is from 6 to 14 nucleobases in length.

TABLE 1 below provides certain non-limiting examples of antisense compounds and their targets:

TABLE 1
Antisense Compounds

| | | | | | SEQ |
|---|---|---|---|---|---|
| | | | | | ID |
| Target | ASO No | Indication | Sequence | Chemistry | NO |
| Factor XI | 416858 | Clotting | ACGGCATTGGTGCACAGTTT | 5-10-5 MOE | 1 |
| | | disorder | | | |
| TTR | 420915 | Amyloidosis | TCTTGGTTACATGAAATCCC | 5-10-5 MOE | 2 |
| Apo(a) | 494372 | CAD | TGCTCCGTTGGTGCTTGTTC | 5-10-5 MOE | 3 |
| Alpha1- | 487660 | Liver disease | CCAGCTCAACCCTTCTTTAA | 5-10-5 MOE | 4 |
| antitrypsin | | | | | |
| PTP-1B | 404173 | Diabetes | AATGGTTTATTCCATGGCCA | 5-10-5 MOE | 5 |
| GCGR | 449884 | Diabetes | GGTTCCCGAGGTGCCCA | 3-10-4 MOE | 6 |
| DGAT2 | 501861 | NASH | TCACAGAATTATCAGCAGTA | 5-10-5 MOE | 7 |
| Factor VII | 540175 | Cancer- | GGACACCCACGCCCCC | 3-10-3 | 8 |
| | | associated | | cEt/MOE | |
| | | thrombosis | | | |
| SMN | 396443 | SMA | TCACTTTCATAATGCTGG | Uniform | 9 |
| | | | | MOE | |
| FGFR4 | 463588 | Obesity | GCACACTCAGCAGGACCCCC | 5-10-5 MOE | 10 |
| apoB-100 | 301012 | High | GCCTCAGTCTGCTTCGCACC | 5-10-5 MOE | 11 |
| | | Cholesterol | | | |
| CRP | 329993 | CAD | AGCATAGTTAACGAGCTCCC | 5-10-5 MOE | 12 |
| ApoC-III | 304801 | High | AGCTTCTTGTCCAGCTTTAT | 5-10-5 MOE | 13 |
| | | triglycerides | | | |
| GCCR | 426115 | Diabetes | GCAGCCATGGTGATCAGGAG | 5-10-5 MOE | 14 |
| STAT3 | 481464 | Cancer | CTATTTGGATGTCAGC | 3-10-3 (S)- | 15 |
| | | | | cEt | |
| eIF-4E | 183750 | Cancer | TGTCATATTCCTGGATCCTT | 5-10-5 MOE | 16 |
| SOD1 | 333611 | ALS | CCGTCGCCCTTCAGCACGCA | 5-10-5 MOE | 17 |
| GHR | 227452 | Acromegaly | TCAGGGCATTCTTTCCATTC | 5-10-5 MOE | 18 |
| Clusterin | 112989 | Cancer | CAGCAGCAGAGTCTTCATCAT | 4-13-4 MOE | 19 |
| Hsp27 | 306053 | Cancer | GGGACGCGGCGCTCGGTCAT | 4-12-4 MOE | 20 |
| CMV | 2922 | Retinitis | GCGTTTGCTCTTCTTCTTGCG | Uniform | 21 |
| | | | | deoxy | |
| ICAM-1 | 2302 | Ulcerative | GCCCAAGCTGGCATCCGTCA | Uniform | 22 |
| | | colitis | | deoxy | |
| VLA-4 | 107248 | Multiple | CTGAGTCTGTTTTCCATTCT | 3-9-8 MOE | 23 |
| | | sclerosis | | | |
| CTGF | 412294 | Fibrosis | GTTTGACATGGCACAATGTT | 2-13-5 MOE | 24 |
| c-raf kinase | 13650 | Ocular disease | TCCCGCCTGTGACATGCATT | 6-8-6 MOE | 25 |

Antisense compounds exert activity through mechanisms involving the hybridization with one or more target nucleic acid, wherein the hybridization results in a biological effect. In certain embodiments, such hybridization results in target nucleic acid degradation and/or occupancy with concomitant inhibition or stimulation of the cellular machinery involving, for example, translation, transcription, splicing or polyadenylation of the target nucleic acid or of a nucleic acid with which the target nucleic acid may otherwise interact.

In certain embodiments, antisense activity results at least in part from degradation of target RNA by RNase H. RNase H is a cellular endonuclease which cleaves the RNA strand of an RNA:DNA duplex. It is known in the art that single-stranded antisense compounds which are DNA or “DNA-like” hybridize to RNA to elicit RNase H mediated activity in mammalian cells. Activation of RNase H, therefore, results in cleavage of the RNA target, thereby greatly enhancing the efficiency of DNA-like oligonucleotide-mediated inhibition of gene expression.

Antisense mechanisms also include, without limitation RNAi mechanisms, which utilize the RISC pathway. Such RNAi mechanisms include, without limitation siRNA, ssRNA and microRNA mechanisms. Such mechanisms include creation of a microRNA mimic and/or an anti-microRNA. To be suitable for RNAi, antisense compounds may be single- or double-stranded and include one or more RNA or RNA-like nucleosides.

In certain embodiments, the target nucleic acid is a pre-mRNA. In certain embodiments, an antisense oligonucleotide modulates splicing of a pre-mRNA. In certain embodiments, antisense compounds alter splicing by hybridizing to a pre-mRNA and disrupting an interaction that is necessary for normal splicing. In certain embodiments, antisense compounds alter splicing by hybridizing to a pre-mRNA and recruiting one or more proteins that elicit splicing.

Antisense mechanisms also include, without limitation, mechanisms that hybridize or mimic non-coding RNA other than microRNA or mRNA. Such non-coding RNA includes, but is not limited to promoter-directed RNA and short and long RNA that effects transcription or translation of one or more nucleic acids.

In certain embodiments, antisense compounds specifically hybridize when there is a sufficient degree of complementarity to avoid non-specific binding of the antisense compound to non-target nucleic acid sequences under conditions in which specific binding is desired, i.e., under physiological conditions in the case of in vivo assays or therapeutic treatment, and under conditions in which assays are performed in the case of in vitro assays.

It is understood in the art that incorporation of nucleotide affinity modifications may allow for a greater number of mismatches compared to an unmodified compound. Similarly, certain oligonucleotide sequences may be more tolerant to mismatches than other oligonucleotide sequences. One of ordinary skill in the art is capable of determining an appropriate number of mismatches between oligonucleotides, or between an oligonucleotide and a target nucleic acid, such as by determining melting temperature (Tₘ). Tₘ or ΔTₘ can be calculated by techniques that are familiar to one of ordinary skill in the art. For example, techniques described in Freier et al. (Nucleic Acids Research, 1997, 25, 22: 4429-4443) allow one of ordinary skill in the art to evaluate nucleotide modifications for their ability to increase the melting temperature of an RNA:DNA duplex.

In certain embodiments, oligomeric compounds of the present invention are RNAi compounds. In certain embodiments, oligomeric compounds of the present invention are ssRNA compounds. In certain embodiments, oligomeric compounds of the present invention are paired with a second oligomeric compound to form an siRNA. In certain such embodiments, the second oligomeric compound is also an oligomeric compound of the present invention. In certain embodiments, the second oligomeric compound is any modified or unmodified nucleic acid. In certain embodiments, the oligomeric compound of the present invention is the antisense strand in an siRNA compound. In certain embodiments, the oligomeric compound of the present invention is the sense strand in an siRNA compound.

### Certain ESCRT Modulator Compounds and Methods

In certain embodiments, modulation of the amount and/or activity of one or more Endosomal Sorting Complex Required for Transport (ESCRT) proteins sensitizes a cell for modulation of a target nucleic acid by antisense compounds. In certain embodiments any compound capable of modulating the amount and/or activity of ESCRT is capable of sensitizing a cell to antisense compounds. Accordingly, ESCRT modulators may be selected from among: antisense compounds directed to ESCRT members, including RNAi and RNase H based antisense compounds directed to ESCRT members, antibodies to ESCRT members, and compounds (e.g., small molecules) capable of binding directly or indirectly to ESCRT members. ESCRT members are divided into four regions: ESCRT-0, which includes, but is not limited to members Hrs, FYVE, UIM, CB, DUBs, Ptdlns(3)P, Clathrin, PSAP, and EPs15b; ESCRT-I, which includes but is not limited to members Vps28, Tsg101, Vps37, Mvb12, UEV, and Alix; ESCRT-II, which includes but is not limited to members Vps22, Vps36, and Vps25; and ESCRT-III, which includes but is not limited to members Vps20, Vps32, Vps24, Vps2 Vps4, Vta1, Vps60, lst1, and Did2. See Raiborg &Stenmark, Nature, 2009, 458, 445-452. Any compound that reduces the amount or activity of any one or more of such members may sensitize a cell to antisense compounds.

Without limiting the present invention by mechanism, it is noted that in certain instances, antisense compounds may be taken into cells by at least two different pathways. In certain such instances, one or more pathway may be productive (results in antisenses activity) and one or more pathway may be non-productive (does not result in antisense activity). In certain such circumstances it is desirable to increase productive uptake and/or decrease non-productive uptake. In certain instances, the Endosomal Sorting Complex Required for Transport (ESCRT) is involved in non-productive uptake. Accordingly, in certain embodiments, reduction in the amount or activity of ESCRT results in a decrease in non-productive uptake of antisense compounds. In certain embodiments, such reduction of non-productive uptake results in increase in productive uptake. In certain such embodiments, the potency of an antisense compound is improved. In certain embodiments, a cell is sensitized for antisense activity by modulating ESCRT activity. In certain embodiments, a cell is sensitized for antisense activity by reducing ESCRT activity. In certain such embodiments, the cell is contacted with an antisense compound. In certain such embodiments, the antisense compound has improved uptake into the cell relative to its uptake in the absence of ESCRT reduction.

Certain excipients designed to increase productive uptake relative to non-productive uptake have been described. See for example WO 2010/091301, which discusses various excipients including, but not limited to polyanions such as dextran sulfate and nucleic acids. In certain embodiments, polyanions such as nonsense nucleic acids may be used to at least partially saturate non-productive uptake to increase the productive uptake of one or more antisense compound. In certain embodiments, such excipients are used together with one or more ESCRT modulator compound. In certain embodiments an excipient and ESCRT modulator and an antisense compound are administered to an animal. In certain embodiments, the excipient and ESCRT modulator and antisense compound are administered to an animal together. In certain embodiments, one or more of the excipient, ESCRT modulator, and antisense compound is administered to an animal separately.

In certain embodiments, an ESCRT modulator is an antisense compound targeting a member of the ESCRT complex. In certain embodiments, such antisense compound targeting a member of the ESCRT complex sensitizes a cell for treatment with an antisense compound. In certain embodiments, the cell is contacted with the ESCRT modulating antisense compound to sensitize it and an antisense compound complementary to a target nucleic acid other than a member of the ESCRT complex, where modulation of the target nucleic acid of that antisense compound is desired. In certain such embodiments, the non-ESCRT targeting antisense compound targets a nucleic acid of biologic interest. In certain such embodiments, the non-ESCRT targeting antisense compound targets a nucleic acid having therapeutic potential. In embodiments in which the ESCRT modulating compound is an antisense compound, it may be selected from any antisense compound described herein (e.g., RNase H activating, RNAi, single- or double-stranded, splice modulator, comprising any modifications and motifs described herein, etc.). In such embodiments, the non-ESCRT modulating antisense compound likewise may be selected from any antisense compound described herein. In embodiments in which the ESCRT modulating compound is not an antisense compound (e.g., antibody or small molecule that modulates ESCRT directly or indirectly) the antisense compound may still be selected from among any antisense compound described herein.

### Certain Compounds and Methods for Improved Cellular Uptake of Antisense Compounds

In certain embodiments, modulation of the amount and/or activity of one or more proteins sensitizes a cell for modulation of a target nucleic acid by antisense compounds. In certain embodiments, modulation of the amount and/or activity of one or more proteins increases the potency of an antisense compound. In certain embodiments, modulation of the amount and/or activity of one or more proteins increases the efficacy of an antisense compound. In certain embodiments, an antisense compound modulates the amount and/or activity of one or more proteins and thereby increases the efficacy of a second antisense compound. In certain embodiments, a non-antisense compound modulates the amount and/or activity of one or more proteins and thereby increases the efficacy of a second antisense compound.

In certain embodiments, modulation of the amount and/or activity of a Low-Density Lipoprotein Receptor (LDL-R) protein sensitizes a cell for modulation of a target nucleic acid by antisense compounds. In certain embodiments, modulation of the amount and/or activity of an LDL-R protein increases the potency of an antisense compound. In certain embodiments, increase of the amount and/or activity of an LDL-R protein increases the potency of an antisense compound. In certain embodiments, administration of one or more statins increases the amount and/or activity of an LDL-R protein. In certain embodiments, administration of one or more statins increases the amount and/or activity of an LDL-R protein and sensitizes a cell for modulation of a target nucleic acid by antisense compounds.

In certain embodiments, an LDL-R modulator is an antisense compound targeting a member of the ESCRT complex. In certain embodiments, such antisense compound targeting a member of the ESCRT complex sensitizes a cell for treatment with an antisense compound. In certain embodiments, the cell is contacted with the ESCRT modulating antisense compound to sensitize it and an antisense compound complementary to a target nucleic acid other than a member of the ESCRT complex, where modulation of the target nucleic acid of that antisense compound is desired. In certain such embodiments, the non-ESCRT targeting antisense compound targets a nucleic acid of biologic interest. In certain such embodiments, the non-ESCRT targeting antisense compound targets a nucleic acid having therapeutic potential. In embodiments in which the ESCRT modulating compound is an antisense compound, it may be selected from any antisense compound described herein (e.g., RNase H activating, RNAi, single- or double-stranded, splice modulator, comprising any modifications and motifs described herein, etc.). In such embodiments, the non-ESCRT modulating antisense compound likewise may be selected from any antisense compound described herein. In embodiments in which the ESCRT modulating compound is not an antisense compound (e.g., antibody or small molecule that modulates ESCRT directly or indirectly) the antisense compound may still be selected from among any antisense compound described herein.

In certain embodiments, an LDL-R modulator is an antisense compound targeting proprotein convertase subtilisin/kexin type 9 (PCSK-9). In certain embodiments, such antisense compound targeting PCSK-9 sensitizes a cell for treatment with an antisense compound. In certain embodiments, the cell is contacted with the PCSK-9 modulating antisense compound to sensitize it and an antisense compound complementary to a target nucleic acid other than PCSK-9, where modulation of the target nucleic acid of that antisense compound is desired. In certain such embodiments, the non-PCSK-9 targeting antisense compound targets a nucleic acid of biologic interest. In certain such embodiments, the non-PCSK-9 targeting antisense compound targets a nucleic acid having therapeutic potential. In embodiments in which the PCSK-9 modulating compound is an antisense compound, it may be selected from any antisense compound described herein (e.g., RNase H activating, RNAi, single- or double-stranded, splice modulator, comprising any modifications and motifs described herein, etc.). In such embodiments, the non-PCSK-9 modulating antisense compound likewise may be selected from any antisense compound described herein. In embodiments in which the PCSK-9 modulating compound is not an antisense compound (e.g., antibody or small molecule that modulates PCSK-9 directly or indirectly) the antisense compound may still be selected from among any antisense compound described herein.

In certain embodiments, the present disclosure provides a method for reducing the amount or activity of a target nucleic acid in a cell comprising contacting a cell with an LDL-R modulator and an antisense compound complementary to the target nucleic acid, wherein the target nucleic acid is other than an ESCRT transcript or a PCSK9 transcript, and wherein the amount or activity of the target nucleic acid in the cell is reduced. In certain embodiments, the target nucleic acid is not a target nucleic acid that encodes Apolipoprotein A, Apolipoprotein B, or Apolipoprotein C-III.

In certain embodiments, an agent is used to increase the amount or activity of LDL-R for the purpose of increasing the potency of an antisense compound. In certain embodiments a small molecule is used to increase the amount or activity of LDL-R. In certain embodiments an antibody is used to increase the amount or activity of LDL-R. In certain embodiments, a statin is used to increase the amount or activity of LDL-R. In certain embodiments, a statin is not used to increase the amount or activity of LDL-R.

In certain embodiments a cell is contacted with a composition comprising an antisense compound and one or more excipients, wherein one or more excipients is a compound that increases the amount of LDL-R activity in a cell. In certain embodiments one or more excipients comprise an antisense compound. In certain embodiments, one or more excipients comprise an antisense compound targeted to PCSK-9. In certain embodiments, one or more excipients comprise a statin. In certain embodiments, none of the excipients comprise a statin.

### Certain Pharmaceutical Compositions

In certain embodiments, the present invention provides pharmaceutical compositions comprising one or more antisense compound. In certain embodiments, the present invention provides pharmaceutical compositions comprising one or more antisense compound and one or more ESCRT modulator. In certain embodiments, such pharmaceutical composition comprises a suitable pharmaceutically acceptable diluent or carrier. In certain embodiments, a pharmaceutical composition comprises a sterile saline solution and one or more antisense compound. In certain embodiments, such pharmaceutical composition consists of a sterile saline solution and one or more antisense compound. In certain embodiments, the sterile saline is pharmaceutical grade saline. In certain embodiments, a pharmaceutical composition comprises one or more antisense compound and sterile water. In certain embodiments, a pharmaceutical composition consists of one or more antisense compound and sterile water. In certain embodiments, the sterile saline is pharmaceutical grade water. In certain embodiments, a pharmaceutical composition comprises one or more antisense compound and phosphate-buffered saline (PBS). In certain embodiments, a pharmaceutical composition consists of one or more antisense compound and sterile phosphate-buffered saline (PBS). In certain embodiments, the sterile saline is pharmaceutical grade PBS.

In certain embodiments, antisense compounds may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

Pharmaceutical compositions comprising antisense compounds encompass any pharmaceutically acceptable salts, esters, or salts of such esters. In certain embodiments, pharmaceutical compositions comprising antisense compounds comprise one or more oligonucleotide which, upon administration to an animal, including a human, is capable of providing (directly or indirectly) the biologically active metabolite or residue thereof. Accordingly, for example, the disclosure is also drawn to pharmaceutically acceptable salts of antisense compounds, prodrugs, pharmaceutically acceptable salts of such prodrugs, and other bioequivalents. Suitable pharmaceutically acceptable salts include, but are not limited to, sodium and potassium salts.

A prodrug can include the incorporation of additional nucleosides at one or both ends of an oligomeric compound which are cleaved by endogenous nucleases within the body, to form the active antisense oligomeric compound.

In certain embodiments, a pharmaceutical composition provided herein comprises a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, a pharmaceutical composition provided herein comprises one or more tissue-specific delivery molecules designed to deliver the one or more pharmaceutical agents of the present invention to specific tissues or cell types. For example, in certain embodiments, pharmaceutical compositions include liposomes coated with a tissue-specific antibody.

In certain embodiments, a pharmaceutical composition provided herein comprises a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, a pharmaceutical composition provided herein is prepared for oral administration. In certain embodiments, pharmaceutical compositions are prepared for buccal administration.

In certain embodiments, a pharmaceutical composition is prepared for administration by injection (e.g., intravenous, subcutaneous, intramuscular, etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions may contain substances that increase the viscosity of the suspension, such as sodium carboxymethyl cellulose, sorbitol, or dextran. Optionally, such suspensions may also contain suitable stabilizers or agents that increase the solubility of the pharmaceutical agents to allow for the preparation of highly concentrated solutions.

In certain embodiments, a pharmaceutical composition is prepared for transmucosal administration. In certain of such embodiments penetrants appropriate to the barrier to be permeated are used in the formulation. Such penetrants are generally known in the art.

In certain embodiments, a pharmaceutical composition provided herein comprises an oligonucleotide in a therapeutically effective amount. In certain embodiments, the therapeutically effective amount is sufficient to prevent, alleviate or ameliorate symptoms of a disease or to prolong the survival of the subject being treated. Determination of a therapeutically effective amount is well within the capability of those skilled in the art.

In certain embodiments, one or more modified oligonucleotide provided herein is formulated as a prodrug. In certain embodiments, upon in vivo administration, a prodrug is chemically converted to the biologically, pharmaceutically or therapeutically more active form of an oligonucleotide. In certain embodiments, prodrugs are useful because they are easier to administer than the corresponding active form. For example, in certain instances, a prodrug may be more bioavailable (e.g., through oral administration) than is the corresponding active form. In certain instances, a prodrug may have improved solubility compared to the corresponding active form. In certain embodiments, prodrugs are less water soluble than the corresponding active form. In certain instances, such prodrugs possess superior transmittal across cell membranes, where water solubility is detrimental to mobility. In certain embodiments, a prodrug is an ester. In certain such embodiments, the ester is metabolically hydrolyzed to carboxylic acid upon administration. In certain instances the carboxylic acid containing compound is the corresponding active form. In certain embodiments, a prodrug comprises a short peptide (polyaminoacid) bound to an acid group. In certain of such embodiments, the peptide is cleaved upon administration to form the corresponding active form.

In certain embodiments, the present invention provides compositions and methods for reducing the amount or activity of a target nucleic acid in a cell. In certain embodiments, the cell is in an animal. In certain embodiments, the animal is a mammal. In certain embodiments, the animal is a rodent. In certain embodiments, the animal is a primate. In certain embodiments, the animal is a non-human primate. In certain embodiments, the animal is a human.

In certain embodiments, the present invention provides methods of administering a pharmaceutical composition comprising an oligomeric compound of the present invention to an animal. Suitable administration routes include, but are not limited to, oral, rectal, transmucosal, intestinal, enteral, topical, suppository, through inhalation, intrathecal, intracerebroventricular, intraperitoneal, intranasal, intraocular, intratumoral, and parenteral (e.g., intravenous, intramuscular, intramedullary, and subcutaneous). In certain embodiments, pharmaceutical intrathecals are administered to achieve local rather than systemic exposures. For example, pharmaceutical compositions may be injected directly in the area of desired effect (e.g., into the eyes, ears).

### Nonlimiting Disclosure and Incorporation by Reference

While certain compounds, compositions and methods described herein have been described with specificity in accordance with certain embodiments, the following examples serve only to illustrate the compounds described herein and are not intended to limit the same. Each of the references, GenBank accession numbers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH for the natural 2′-H of DNA) or as an RNA having a modified base (thymine (methylated uracil) for natural uracil of RNA).

Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified or naturally occurring bases, such as “AT^{me}CGAUCG,” wherein ^{me}C indicates a cytosine base comprising a methyl group at the 5-position.

### EXAMPLES

The following examples illustrate certain embodiments of the present invention and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif. And, for example, where a particular high-affinity modification appears at a particular position, other high-affinity modifications at the same position are considered suitable, unless otherwise indicated.

### Example 1

### Evaluation of Functional Uptake of Single Stranded Antisense Oligonucleotides (ASOs) Targeting SR-B1 in the Presence of Vps28 Inhibitor

A single stranded antisense oligonucleotide (ASO) was evaluated for its functional uptake in MHT cells (Mouse Hepatocellular carcinoma cell line) or b.END cells in the presence and absence of Vps28 inhibitor. Vps28 (Vacuolar protein sorting-associated protein 28 homolog) is a member of the ESCRT complex (Endosome Sorting Complex Required For Transport).

ASO 353382 (a 5-10-5 MOE-DNA-MOE gapmer having all phosphorothioate linkages and a nucleobase sequence complementary to SR-B1), was prepared using the procedures published in the literature (Koller et al., Nucleic Acids Res., 2011, 39(11), 4795-47807). Two Vps28 modulators were tested. As shown in Table 3, each Vps28 modulator was an siRNA targeted to Vps28. All siRNAs were purchased from Dharmacon Research Inc. (Boulder, Colo., USA).

The ASO and siRNAs are described in Table 3. A subscript “s” between two nucleosides indicates a phosphorothioate internucleoside linkage (going 5′ to 3′ or 3′ to 5′). The absence of a subscript “s” between two nucleosides indicates a phosphodiester internucleoside linkage. Nucleosides without a subscript are ribonucleosides (RNA). Nucleosides with subscripts “d” are β-D-2′-deoxyribonucleosides. A subscript “e” indicates a 2′-O-methoxyethyl (MOE) modified nucleoside. ^{me}C indicates a 5-methyl cytosine nucleoside.

Quantitative RT-PCR (qRT-PCR)

Total mRNA was isolated using a QIAGEN RNAeasy kit (QIAGEN, Valencia, Calif., USA). Reduction of target mRNA expression was determined by qRT-PCR using StepOne RT-PCR machines (Applied Biosystems). The sequences for primers and probes used in RT-PCR reaction are presented in Table 2. The expression data was normalized to RIBOGREEN (Invitrogen). Mean values±SDs of three replicates are provided in Table 4.

TABLE 2
Primers and Probes

| Target | Forward (5′ to 3′) | Reverse (5′ to 3 ) | Probe (5′ to 3′) | SEQ ID No. |
|---|---|---|---|---|
| SR B1 | TGACAACGACACCGT | ATGCGACTTGTCAG | CGTGGAGAACCGCAGCC | 26, 27, 28 |
| | GTCCT | GCTGG | TCCATT | |
| PTEN | GCCACAGGCTCCCAG | TCCATCCTCTTGAT | ACAGCCATCATCAAAGA | 29, 30, 31 |
| | ACAT | ATCTCCTTTTG | GATCGTTAGCAGAA | |
| Malat1 | TGGGTTAGAGAAG | TCAGCGGCAACT | CGTTGGCACGACACCT | 32, 33, 34 |
| | GCGTGTACTG | GGGAAA | TCAGGGACT | |
| Vps28 | CTTCGATCTGGAGTC | TTCCTGTCTCGGT | CGTTGGCACGACACCT | 35, 36, 37 |
| | CGCTTA | GAGGCTTA | TCAGGGACT | |

### Cell Culture and Transfection

MHT cells were isolated from a hepatocellular carcinoma tumor which developed in transgenic mouse expressing SV40 large T-antigen under the CRP promoter (Ruther et al., Oncogene, 1993, 8, 87-93) and cultured in DMEM supplemented with 10% fetal bovine serum (FBS), streptomycin (0.1 ug/mL), and penicillin (100 U/mL). b.END cells were obtained from ATCC and cultured in DMEM containing 10% fetal bovine serum.

To characterize the uptake of ASO in the presence of Vps28 inhibitor, cultured MHT cells or b.END cells were treated with one of two different Vps28 siRNAs or neg control siRNA complementary to no target and ASO 353382 targeting scavenger receptor B1 (SR-B1). Cells were plated at a density of 7,500 cells per 96-well and transfected using Opti-MEM containing 5 ug/mL Lipofectamine 2000. First transfection was performed using 40 nM concentration of Vps28 siRNAs or negative control siRNA. These siRNAs are denoted as “Vps28 siRNA-1” or “Vps28 siRNA-3” for Vps28 inhibitors and “Con siRNA” for negative control. After a treatment period of 4 hrs, transfection medium was replaced with complete growth medium and a second transfection was performed 24 hrs later in the same manner as above. 24 hrs later, ASO 353382 was added to complete growth medium (DMEM, 10% FBS) at concentrations listed in Table 4. RNA was isolated from cells after 24 hours and SR-B1 mRNA levels were measured by qRT-PCR as described above.

As illustrated in Table 4, an increase in reduction of SR-B1 mRNA levels was observed in MHT and b.END cells for ASO 353382 in the presence of Vps28 inhibitor as compared to the negative control. The results demonstrate that inhibition of Vps28 increases the potency of ASO 353382. As expected, treatment with Vps28 siRNA reduced Vps28 mRNA levels in MHT and b.END cells (data not shown).

TABLE 3
ASO targeting SR-B1 and siRNAs targeting Vps28

| RNA | Oligo No. | Composition | SEQ ID No. |
|---|---|---|---|
| ASO | 353382 | 5′-Gₑₛ^{m}CₑₛTₑₛTₑₛ^{m}CₑₛA_{ds}G_{ds}Tₛₛ^{m}C_{ds}A_{ds}T_{ds}G_{ds} | 38 |
| | | A_{ds}^{m}C_{ds}T_{ds}Tₑₛ^{m}Cₑₛ^{m}CₑₛTₑₛTₑ-3′ | |
| siRNA | Vps28 siRNA-1 | 5′-UCGGAAGGCAGCUUUGUACUU-3′ | 39 |
| | | 3′-UUAGCCUUCCGUCGAAACAUG-5′ | 40 |
| siRNA | Vps28 siRNA-3 | 5′-GAAGUAAAGCUCUACAAGAUU-3′ | 41 |
| | | 3′-UCCUUCAUUUCGAGAUGUUCU-5′ | 42 |
| siRNA | Con siRNA | 5′-UACAUAACCGGACAUAAUCUU-3′ | 43 |
| | (neg control) | 3′-UUAUGUAUUGGCCUGUAUUAG-5′ | 44 |

TABLE 4
Inhibition of SR-B1 mRNA level with ASO in the
presence of Vps28 inhibitor in MHT cells

| siRNA + ASO | | SR-B1 mRNA level |
|---|---|---|
| Treatment | Conc. of ASO (nM) | (% control) |
| Vps28 siRNA-1 + | 10000 | 12.08 |
| ASO 353382 | 2000 | 17.27 |
| | 400 | 20.76 |
| | 80 | 34.77 |
| | 16 | 66.9 |
| | 3.2 | 104.78 |
| | 0.64 | 129.86 |
| | 0.16 | 139.65 |
| Vps28 siRNA-3 + | 10000 | 14.01 |
| ASO 353382 | 2000 | 19.22 |
| | 400 | 27.06 |
| | 80 | 40.41 |
| | 16 | 68.04 |
| | 3.2 | 113.86 |
| | 0.64 | 129.94 |
| | 0.16 | 128.26 |
| Con siRNA | 10000 | 25.71 |
| (neg control) + | 2000 | 41.58 |
| ASO 353382 | 400 | 54.91 |
| | 80 | 60.86 |
| | 16 | 85.08 |
| | 3.2 | 114.57 |
| | 0.64 | 112.28 |
| | 0.16 | 115.55 |

TABLE 5
Inhibition of SR-B1 mRNA level with ASO in the
presence of Vps28 inhibitor in b.END cells

| siRNA + ASO | | SR-B1 mRNA level |
|---|---|---|
| Treatment | Conc. of ASO (nM) | (% control) |
| Vps28 siRNA-1 + | 10000 | 13.63 |
| ASO 353382 | 2000 | 18.61 |
| | 400 | 17.03 |
| | 80 | 22.29 |
| | 16 | 37.82 |
| | 3.2 | 62.76 |
| | 0.64 | 77.56 |
| | 0.16 | 91.16 |
| Vps28 siRNA-3 + | 10000 | 4.4 |
| ASO 353382 | 2000 | 6.62 |
| | 400 | 9.6 |
| | 80 | 15.45 |
| | 16 | 33.18 |
| | 3.2 | 58.1 |
| | 0.64 | 76.77 |
| | 0.16 | 85.42 |
| Con siRNA | 10000 | 12.55 |
| (neg control) + | 2000 | 20.29 |
| ASO 353382 | 400 | 34.3 |
| | 80 | 40.92 |
| | 16 | 81.31 |
| | 3.2 | 96.2 |
| | 0.64 | 104.56 |
| | 0.16 | 96.46 |

### Example 2

### Evaluation of Functional Uptake of ASOs Targeting PTEN, SR-B1, or Malat1 in the Presence of Vps28 Inhibitor

ASOs and siRNAs

ASOs 353382, 116847 and 399479 targeting PTEN, SRB-1 and Malat1, respectively, were evaluated for functional uptake in MHT cells in the presence of Vps28 inhibitor.

The ASOs were prepared using the procedures published in the literature (Koller et al., Nucleic Acids Res., 2011, 39(11), 4795-47807) and the siRNAs were purchased from Dharmacon Research Inc. (Boulder, Colo., USA).

The ASOs and siRNA are described in Table 6. A subscript “s” between two nucleosides indicates a phosphorothioate internucleoside linkage (going 5′ to 3′ or 3′ to 5′). The absence of a subscript “s” between two nucleosides indicates a phosphodiester internucleoside linkage. Nucleosides without a subscript are ribonucleosides (RNA). Nucleosides with subscripts “d” are β-D-2′-deoxyribonucleosides. A subscript “e” indicates a 2′-O-methoxyethyl (MOE) modified nucleoside. ^{me}C indicates a 5-methyl cytosine nucleoside.

### Cell Culture and Transfection

MHT cells were cultured in the same manner as described in Example 1. To characterize the uptake of ASOs in the presence of Vps28 inhibitor, cultured MHT cells were treated with Vps28 siRNA or neg control siRNA and ASO targeting PTEN, SR-B1 or Malat1. Cells were plated at a density of 7,500 cells per well and transfected using Opti-MEM containing 5 ug/mL Lipofectamine 2000. First transfection was performed using 40 nM concentration of Vps28 siRNA or negative control siRNA. These siRNAs are denoted as “Vps28 siRNA-3” for Vps28 inhibitor and “Con siRNA” for negative control. After a treatment period of 4 hours, transfection medium was replaced with complete growth medium and a second transfection was performed 24 hrs later in the same manner as described above. 24 hrs later, ASO 353382, 116847 or 399479 was added to complete growth medium (DMEM, 10% FBS) at concentrations listed in Table 7. RNA was isolated from cells after 24 hours and target mRNA levels were measured by qRT-PCR utilizing the method described in Example 1.

### Analysis of IC₅₀'s

The half maximal inhibitory concentrations (IC₅₀) of ASOs were calculated by plotting the concentrations of oligonucleotides versus the percent inhibition of PTEN, SR-B1 or Malat1 mRNA expression achieved at each concentration, and noting the concentration of oligonucleotides at which 50% inhibition of PTEN, SR-B1 or Malat1 mRNA expression was achieved compared to the negative control. The results are presented is presented in Table 7 below.

As illustrated in Table 7, Vps28 inhibition by siRNA increased in reduction of target mRNA levels for ASOs compared to the negative control in which Vps28 was not inhibited. The results demonstrate that inhibition of Vps28 sensitizes cells for ASO treatment.

TABLE 6
ASO targeting PTEN, SR-B1 or Malat1 and siRNAs targeting Vps28

| | | | | SEQ |
|---|---|---|---|---|
| RNA | Oligo No | Composition | Target | ID No. |
| ASO | 116847 | 5′-^{m}CₑₛTₑₛGₑₛ^{m}CₑₛTₑₛA_{ds}G_{ds}^{m}C_{ds}^{m}C_{ds}T_{ds} | PTEN | 45 |
| | | ^{m}C_{ds}T_{ds}G_{ds}G_{ds}A_{ds}TₑₛTₑₛTₑₛGₑₛAₑ-3′ | | |
| ASO | 353382 | 5′-Gₑₛ^{m}CₑₛTₑₛTₑₛ^{m}CₑₛA_{ds}G_{ds}T_{ds}^{m}C_{ds}A_{ds} | SR-B1 | 38 |
| | | T_{ds}G_{ds}A_{ds}^{m}C_{ds}T_{ds}Tₑₛ^{m}Cₑₛ^{m}CₑₛTₑₛTₑ-3′ | | |
| ASO | 399479 | 5′-^{m}CₑₛGₑₛGₑₛTₑₛGₑₛ^{m}C_{ds}A_{ds}A_{ds}G_{ds}G_{ds} | Malat1 | 46 |
| | | ^{m}C_{ds}T_{ds}T_{ds}A_{ds}G_{ds}GₑₛAₑₛAₑₛTₑₛTₑ-3′ | | |
| si | Vps 28 siRNA-3 | 5′-GAAGUAAAGCUCUACAAGAUU-3′ | Vps28 | 41 |
| | | 3′-UCCUUCAUUUCGAGAUGUUCU-5′ | | 42 |
| si | Con siRNA | 5′-UACAUAACCGGACAUAAUCUU-3′ | Luciferase | 43 |
| | (neg control) | 3′-UUAUGUAUUGGCCUGUAUUAG-5′ | | 44 |

TABLE 7
Inhibition of PTEN, SR-B1 or Malat1 mRNA
levels with ASOs in the presence of Vps28 inhibitor

| | | | IC₅₀ |
|---|---|---|---|
| siRNA + ASO Treatment | Conc. of ASO (nM) | Target | (nM) |
| Vps28 siRNA-3 + | 10,000, 2,000, 400, 80, | PTEN | 200 |
| ASO 116847 | 16, 3.2, 0.64, 0.16 | | |
| Con siRNA | | | >100,000 |
| (neg control) + | | | |
| ASO 116847 | | | |
| Vps28 siRNA-3 + | 10,000, 2,000, 400, 80, | SR-B1 | 8 |
| ASO 353382 | 16, 3.2, 0.64, 0.16 | | |
| Con siRNA | | | 80 |
| (neg control) + | | | |
| ASO 353382 | | | |
| Vps28 siRNA-3 + | 100, 20, 4, 0.8 | Malat1 | 2 |
| ASO 399479 | | | |
| Con siRNA | | | 20 |
| (neg control) + | | | |
| ASO 399479 | | | |

### Example 3

### Evaluation of Functional Uptake of ASOs Targeting SR-B1 in the Presence of Mvb12b Inhibitor

ASOs and siRNAs

The ASO 353382 from Table 3 was evaluated for its functional uptake in MHT cells or b.END cells in the presence of Mvb12b inhibitor. Mvb12b is another member of the ESCRT pathway that may be involved in the functional uptake of ASOs.

The ASO 353382 was prepared using the procedures published in the literature (Koller et al., Nucleic Acids Res., 2011, 39(11), 4795-47807) and the siRNAs were purchased from Life Technologies, Carlsbad, Calif., USA)

The ASOs and siRNA are described in Table 8. A subscript “s” between two nucleosides indicates a phosphorothioate internucleoside linkage (going 5′ to 3′ or 3′ to 5′). The absence of a subscript “s” between two nucleosides indicates a phosphodiester internucleoside linkage. Nucleosides without a subscript are ribonucleosides (RNA). Nucleosides with subscripts “d” are β-D-2′-deoxyribonucleosides. A subscript “e” indicates a 2′-O-methoxyethyl (MOE) modified nucleoside. ^{me}C indicates a 5-methyl cytosine nucleoside.

### Cell Culture and Transfection

MHT and b.END cells were cultured utilizing the method described in Example 1. To further characterize the uptake of ASO in the presence of Mvb12b inhibitor, cultured MHT cells or b.END cells were treated with Mvb12b siRNA or neg control siRNA and ASO 353382 targeting SR-B1. Cells were plated at a density of 7,500 cells per 96-well and transfected using Opti-MEM containing 5 ug/mL Lipofectamine 2000. First transfection was performed using 40 nM concentration of Mvb12b siRNA or negative control siRNA. The siRNA is denoted as “Mvb12b siRNA” for Mvb12b inhibitor and “Con siRNA” for negative control. After a treatment period of 4 hrs, transfection medium was replaced with complete growth medium and a second transfection was performed 24 hrs later in the same manner as described above. 24 hrs later, ASO 353382 was added to complete growth medium (DMEM, 10% FBS) at concentrations listed in Tables 9 and 10. RNA was isolated from cells after 24 hours and SR-B1 mRNA levels were measured by qRT-PCR as described in Example 1.

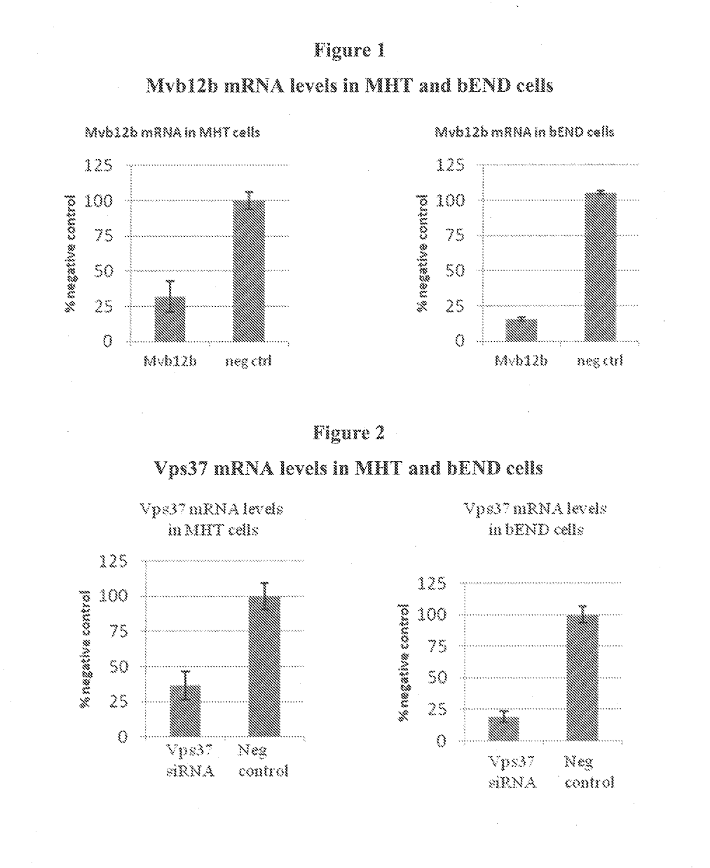

As illustrated in Tables 9 and 10, an increase in reduction of SR-B1 mRNA levels was observed in MHT and b.END cells for ASO 353382 in the presence of Mvb12b inhibitor as compared to the negative control. The results demonstrate that inhibition of Mvb12b increases the potency of ASO 353382. As expected, treatment with Mvb12b siRNA reduced Mvb12b mRNA levels in MHT and b.END cells (FIG. 1).

TABLE 8
ASOs targeting SR-Bland siRNAs targeting Mvb12b

| RNA | Oligo No. | Composition | SEQ ID No. |
|---|---|---|---|
| ASO | 353382 | 5′-Gₑₛ^{m}CₑₛTₑₛTₑₛ^{m}CₑₛA_{ds}G_{ds}T_{ds}^{m}C_{ds}A_{ds} | 38 |
| | | T_{ds}G_{ds}A_{ds}^{m}C_{ds}T_{ds}Tₑₛ^{m}Cₑₛ^{m}CₑₛTₑₛTₑ-3′ | |
| siRNA | Mvb12b siRNA | 5′-GGUUACCAGAUACCUGUGUUU-3′ | 47 |
| | | 3′-UUCCAAUGGUCUAUGGACACA-5′ | 48 |
| siRNA | Con siRNA | 5′-UACAUAACCGGACAUAAUCUU-3′ | 43 |
| | (neg control) | 3′-UUAUGUAUUGGCCUGUAUUAG-5′ | 44 |

TABLE 9
Inhibition of SR-B1 mRNA level with ASO in the
presence of Mvb12b inhibitor in MHT cells

| | | SR-B1 mRNA level |
|---|---|---|
| siRNA + ASO Treatment | Conc. of ASO (nM) | (% control) |
| Mvb12b siRNA + | 10000 | 10.29 |
| ASO 353382 | 2000 | 18.19 |
| | 400 | 26.24 |
| | 80 | 29.5 |
| | 16 | 52.7 |
| | 3.2 | 92.83 |
| | 0.64 | 86.96 |
| | 0.16 | 116.85 |
| Con siRNA | 10000 | 25.71 |
| (neg control) + | 2000 | 41.58 |
| ASO 353382 | 400 | 54.91 |
| | 80 | 60.86 |
| | 16 | 85.08 |
| | 3.2 | 114.57 |
| | 0.64 | 112.28 |
| | 0.16 | 115.55 |

TABLE 10
Inhibition of SR-B1 mRNA level with ASO in the
presence of Mvb12b inhibitor in b.END cells

| siRNA + ASO | Conc. of ASO | SR-B1 mRNA level |
|---|---|---|
| Treatment | (nM) | (% control) |
| Mvb12b siRNA + | 10000 | 22.75 |
| ASO 353382 | 2000 | 28.66 |
| | 400 | 34.59 |
| | 80 | 42.38 |
| | 16 | 48.09 |
| | 3.2 | 63.12 |
| | 0.64 | 71.9 |
| | 0.16 | 70.17 |
| Con siRNA | 10000 | 40.88 |
| (neg control) + | 2000 | 53.17 |
| ASO 353382 | 400 | 66.17 |
| | 80 | 68.53 |
| | 16 | 72.93 |
| | 3.2 | 95.32 |
| | 0.64 | 105.32 |
| | 0.16 | 98.55 |

### Example 4

### Evaluation of Functional Uptake of ASOs Targeting SR-B1 in the Presence of Vps37 Inhibitor

ASOs and siRNAs

ASO 353382 from Table 3 was selected and evaluated for its functional uptake in MHT cells or b.END cells in the presence of Vps37 inhibitor. Vps37 is another member of the ESCRT pathway that may be involved in the functional uptake of ASOs.

ASO 3533382 was prepared using the procedures published in the literature (Koller et al., Nucleic Acids Res., 2011, 39(11), 4795-47807) and the siRNAs were purchased from Dharmacon Research Inc. (Boulder, Colo., USA).

The ASO and siRNAs are described in Table 11. A subscript “s” between two nucleosides indicates a phosphorothioate internucleoside linkage (going 5′ to 3′ or 3′ to 5′). The absence of a subscript “s” between two nucleosides indicates a phosphodiester internucleoside linkage. Nucleosides without a subscript are ribonucleosides (RNA). Nucleosides with subscripts “d” are β-D-2′-deoxyribonucleosides. A subscript “e” indicates a 2′-O-methoxyethyl (MOE) modified nucleoside. ^{me}C indicates a 5-methyl cytosine nucleoside.

### Cell Culture and Transfection

MHT and b.END cells were cultured utilizing the method described in Example 1. To further characterize the uptake of ASO in the presence of Vps37 inhibitor, cultured MHT cells or b.END cells were treated Vps37 siRNA or neg control siRNA and ASO 353382 targeting SR-B1. Cells were plated at a density of 20,000 cells per well and transfected using Opti-MEM containing 5 ug/mL Lipofectamine 2000. First transfection was performed using 40 nM concentration of Vps37 siRNA or negative control siRNA. The siRNA is denoted as “Vps37 siRNA” for Vps37 inhibitor and “Con siRNA” for negative control. After a treatment period of 4 hrs, transfection medium was replaced with complete growth medium and a second transfection was performed 24 hrs later in the same manner as described above. 24 hrs later, ASO 353382 was added to complete growth medium (DMEM, 10% FBS) at concentrations listed in Tables 12 and 13. RNA was isolated from cells after 24 hours and SR-B1 mRNA levels were measured by qRT-PCR as described in Example 1.

As illustrated in Tables 12 and 13, an increase in reduction of SR-B1 mRNA levels was observed in MHT and b.END cells for ASO 353382 in the presence of Vps37 inhibitor as compared to the negative control. The results demonstrate that inhibition of Vps37 increases the potency of ASO 353382. As expected, treatment with Vps37 siRNA reduced Vps37 mRNA levels in MHT and b.END cells (FIG. 2).

TABLE 11
ASOs targeting SR-Bland siRNAs targeting Vps37

| RNA | Oligo No. | Composition | SEQ ID No. |
|---|---|---|---|
| ASO | 353382 | 5′-Gₑₛ^{m}CₑₛTₑₛTₑₛ^{m}CₑₛA_{ds}G_{ds}T_{ds}^{m}C_{ds}A_{ds} | 38 |
| | | T_{ds}G_{ds}A_{ds}^{m}C_{ds}T_{ds}Tₑₛ^{m}Cₑₛ^{m}CₑₛTₑₛTₑ-3 | |
| siRNA | Vps37 siRNA | 5′-GGCAAACCGUUUUAGAUAAUU-3′ | 49 |
| | | 3′-UUCCGUUUGGCAAAAUCUAUU-5′ | 50 |
| siRNA | Con siRNA | 5′-UACAUAACCGGACAUAAUCUU-3′ | 43 |
| | (neg control) | 3′-UUAUGUAUUGGCCUGUAUUAG-5′ | 44 |

TABLE 12
Inhibition of SR-B1 mRNA level with ASO in
the presence of Vps37 inhibitor in MHT cells

| | | SR-B1 mRNA level |
|---|---|---|
| siRNA + ASO Treatment | Conc. of ASO (nM) | (% control) |
| Vps37 siRNA + | 10000 | 22.85 |
| ASO 353382 | 2000 | 30.27 |
| | 400 | 39.53 |
| | 80 | 44.85 |
| | 16 | 75.6 |
| | 3.2 | 80.91 |
| | 0.64 | 105.38 |
| | 0.16 | 108.28 |
| Con siRNA | 10000 | 25.71 |
| (neg control) + | 2000 | 41.58 |
| ASO 353382 | 400 | 54.91 |
| | 80 | 60.86 |
| | 16 | 85.08 |
| | 3.2 | 114.57 |
| | 0.64 | 112.28 |
| | 0.16 | 115.55 |

TABLE 13
Inhibition of SR-B1 mRNA level with ASO in the
presence of Vps37 inhibitor in b.END cells

| siRNA + ASO | Conc. of | SR-B1 mRNA level |
|---|---|---|
| Treatment | ASO (nM) | (% control) |
| Vps37 siRNA + | 10000 | 14.6 |
| ASO 353382 | 2000 | 22.09 |
| | 400 | 32.42 |
| | 80 | 42.3 |
| | 16 | 56.97 |
| | 3.2 | 78.66 |
| | 0.64 | 76.54 |
| | 0.16 | 93.97 |
| Con siRNA | 10000 | 12.55 |
| (neg control) + | 2000 | 20.29 |
| ASO 353382 | 400 | 34.3 |
| | 80 | 40.92 |
| | 16 | 81.31 |
| | 3.2 | 96.2 |
| | 0.64 | 104.56 |
| | 0.16 | 96.46 |

### Example 5

### Evaluation of Functional Uptake of ASOs Targeting SR-B1 in the Presence of Tsg101 Inhibitor

ASOs and siRNAs

ASO 353382 from Table 3 was selected and evaluated for its functional uptake in MHT cells or b.END cells in the presence of Tsg101 inhibitor. Tsg101 is another member of the ESCRT pathway that may be involved in the functional uptake of ASOs.

ASO 353382 was prepared using the procedures published in the literature (Koller et al., Nucleic Acids Res., 2011, 39(11), 4795-47807) and the siRNAs were purchased from Dharmacon Research Inc. (Boulder, Colo., USA).

The ASO and siRNAs are described in Table 14. A subscript “s” between two nucleosides indicates a phosphorothioate internucleoside linkage (going 5′ to 3′ or 3′ to 5′). The absence of a subscript “s” between two nucleosides indicates a phosphodiester internucleoside linkage. Nucleosides without a subscript are ribonucleosides (RNA). Nucleosides with subscripts “d” are β-D-2′-deoxyribonucleosides. A subscript “e” indicates a 2′-O-methoxyethyl (MOE) modified nucleoside. ^{me}C indicates a 5-methyl cytosine nucleoside.

### Cell Culture and Transfection

MHT and b.END cells were isolated and cultured utilizing the method described in Example 1. To further characterize the uptake of ASO in the presence of Tsg101 inhibitor, cultured MHT cells or b.END cells were treated with two different Tsg101 siRNAs or neg control siRNA and ASO 353382 targeting SR-B1. Cells were plated at a density of 7,500 cells per 96-well and transfected using Opti-MEM containing 5 ug/mL Lipofectamine 2000. First transfection was performed using 40 nM concentration of Tsg101 siRNA or negative control siRNA. The siRNA is denoted as “Tsg101 siRNA-1” or “Tsg101 siRNA-3” for Tsg101 inhibitors and “Con siRNA” for negative control. After a treatment period of 4 hrs, transfection medium was replaced with complete growth medium and a second transfection was performed 24 hrs later in the same manner as described above. 24 hrs later, ASO 353382 was added to complete growth medium (DMEM, 10% FBS) at concentrations listed in Tables 15 and 16. RNA was isolated from cells after 24 hours and SR-B1 mRNA levels were measured by qRT-PCR as described in Example 1.

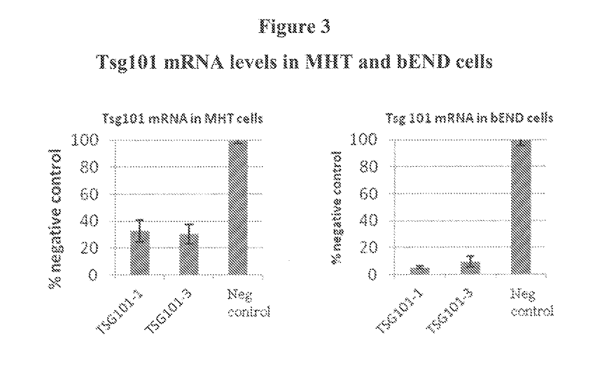

As illustrated in Tables 15 and 16, an increase in reduction of SR-B1 mRNA levels was observed in MHT and b.END cells for ASO353382 in the presence of Tsg101 inhibitor as compared to the negative control. The results demonstrate that inhibition of Tsg101 increases the potency of ASO 353382. As expected, treatment with Tsg101 siRNA reduced Tsg101 mRNA levels in MHT and b.END cells (FIG. 3).

TABLE 14
ASOs targeting SR-B1 and siRNAs targeting Tsg101

| RNA | Oligo No. | Composition | SEQ ID No. |
|---|---|---|---|
| ASO | 353382 | 5′-Gₑₛ^{m}CₑₛTₑₛTₑₛ^{m}CₑₛA_{ds}G_{ds}T_{ds}^{m}C_{ds}A_{ds} | 38 |
| | | T_{ds}G_{ds}A_{ds}^{m}C_{ds}T_{ds}Tₑₛ^{m}Cₑₛ^{m}CₑₛTₑₛTₑ-3 | |
| siRNA | Tsg101 siRNA-1 | 5′-UACUUCUUGAUCUAAGCGGUU-3′ | 51 |
| | | 3′-UUAUGAAGAACUAGAUUCGCC-5′ | 52 |
| siRNA | Tsg101 siRNA-3 | 5′-UAACGCACUGGGAUUGUACUU-3′ | 53 |
| | | 3′-UUAUUGCGUGACCCUAACAUG-5′ | 54 |
| siRNA | Con siRNA | 5′-UACAUAACCGGACAUAAUCUU-3′ | 43 |
| | (neg control) | 3′-UUAUGUAUUGGCCUGUAUUAG-5′ | 44 |

TABLE 15
Inhibition of SR-B1 mRNA level with ASO in the
presence of Tsg101 inhibitor in MHT Cells

| siRNA + ASO | Conc. of | SR-B1 mRNA level |
|---|---|---|
| Treatment | ASO (nM) | (% control) |
| Tsg101 siRNA-1 + | 10000 | 14.76 |
| ASO 353382 | 2000 | 17.84 |
| | 400 | 26.34 |
| | 80 | 37.7 |
| | 16 | 62.68 |
| | 3.2 | 91.64 |
| | 0.64 | 110.07 |
| | 0.16 | 124.06 |
| Tsg101 siRNA-3 + | 10000 | 17.23 |
| ASO 353382 | 2000 | 26.24 |
| | 400 | 35.79 |
| | 80 | 46.78 |
| | 16 | 75.7 |
| | 3.2 | 110.79 |
| | 0.64 | 117.73 |
| | 0.16 | 122.31 |
| Con siRNA | 10000 | 25.71 |
| (neg control) + | 2000 | 41.58 |
| ASO 353382 | 400 | 54.91 |
| | 80 | 60.86 |
| | 16 | 85.08 |
| | 3.2 | 114.57 |
| | 0.64 | 112.28 |
| | 0.16 | 115.55 |

TABLE 16
Inhibition of SR-B1 mRNA level with ASO in the
presence of Tsg101 inhibitor in b.END cells

| siRNA + ASO | Conc. of | SR-B1 mRNA level |
|---|---|---|
| Treatment | ASO (nM) | (% control) |
| Tsg101 siRNA-1 + | 10000 | 11.14 |
| ASO 353382 | 2000 | 15.26 |
| | 400 | 19.56 |
| | 80 | 24.96 |
| | 16 | 36.96 |
| | 3.2 | 50.66 |
| | 0.64 | 49.19 |
| | 0.16 | 54.27 |
| Tsg101 siRNA-3 + | 10000 | 14.1 |
| ASO 353382 | 2000 | 17.44 |
| | 400 | 21.48 |
| | 80 | 29.14 |
| | 16 | 35.05 |
| | 3.2 | 53.16 |
| | 0.64 | 70.83 |
| | 0.16 | 78.91 |
| Con siRNA | 10000 | 12.55 |
| (neg control) + | 2000 | 20.29 |
| ASO 353382 | 400 | 34.3 |
| | 80 | 40.92 |
| | 16 | 81.31 |
| | 3.2 | 96.2 |
| | 0.64 | 104.56 |
| | 0.16 | 96.46 |

### Example 6

### Effect of Vps28 and Tsg101 Depletion on EGFR Degradation

siRNAs

siRNAs were selected and evaluated for the effect of Vps28 and Tsg101 depletion on EGFR (Epidermal Growth Factor Receptor) degradation. Tsg101 depletion has been shown to inhibit EGFR degradation. Vps28 is in ESCRT-I like Tsg101 and inhibition of Vps28 has the same effect as inhibition of Tsg101.

The siRNAs are commercially available from Dharmacon Research Inc. (Boulder, Colo., USA) and are described in Table 17. The internucleoside linkages throughout the siRNA are phosphodiester internucleoside linkage (P═O). Nucleosides without a subscript are ribonucleosides (RNA).

### Cell Culture and Transfection

MHT cells were isolated and cultured utilizing the method described in Example 1. To evaluate the effect of Vps28 and Tsg101 depletion on EGFR degradation, cultured MHT cells were treated Vps28, Tsg101 or Luciferase siRNAs. Cells were plated at a density of 20,000 cells per well and transfected using Opti-MEM containing 5 ug/mL Lipofectamine 2000 at 40 nM concentration of Tsg101 siRNA-1, Tsg siRNA-2, Vps28 siRNA-3, or negative control. Luciferase siRNA was used as a negative control. After a treatment period of 4 hrs, transfection medium was replaced with complete growth medium and a second transfection was performed 24 hrs later in the same manner as described above. Cells were serum starved overnight and then treated with 10 μg/ml cyclohexamide in serum free medium for 60 minutes. Cells were then treated with 200 ng/ml EGF and lysed at 0, 20, 60, 120, 180, and 240 minutes later. EGFR protein analysis by Western blots and quantitation relative to negative control were performed utilizing the method described below. Mean EGFR protein levels are shown from three independent experiments.

### Western Blotting

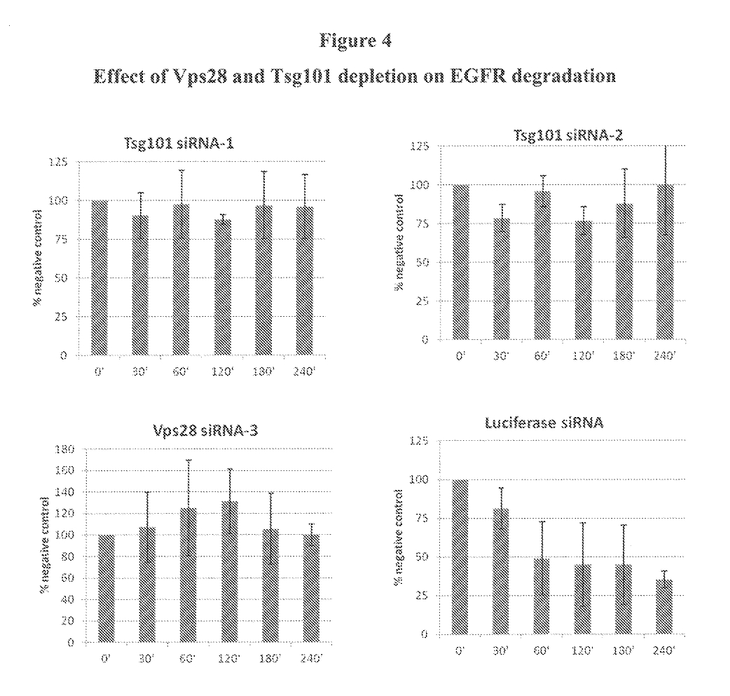

Cells were lysed in RIPA lysis buffer. Equal amounts of protein were resolved on a SDS-PAGE gel and transferred to membranes. Proteins were detected using EGFR antibodies from Abcam (Cambridge, Mass., USA). Secondary antibodies (Lincoln, Nebr., USA) were conjugated to IR800. Blots were scanned using Odyssey from LI-COR. Protein bands were quantified using Li-Cor software. Mean results from three independent experiments are presented in FIG. 4 and demonstrate that depletion of Vps28 or Tsg101 inhibits the degradation of EGFR compared to negative control in which Vps28 and Tsg101 were not depleted. It has been shown that inhibiting other ESCRT-I proteins such as Tsg101 inhibits degradation of EGFR. Vps28 is in ESCRT-I complex like Tsg101 and thus, inhibition of Vps28 has the same effect as inhibition of Tsg101.

TABLE 17
siRNAs

| RNA | Oligo No. | Composition | SEQ ID No. |
|---|---|---|---|
| siRNA | Tsg101 siRNA-1 | 5′-UACUUCUUGAUCUAAGCGGUU-3′ | 51 |
| | | 3′-UUAUGAAGAACUAGAUUCGCC-5′ | 52 |
| siRNA | Tsg101 siRNA-2 | 5′-UAACGCACUGGGAUUGUACUU-3′ | 53 |
| | | 3′-UUAUUGCGUGACCCUAACAUG-5′ | 54 |
| siRNA | Vps28 siRNA-3 | 5′-GAAGUAAAGCUCUACAAGAUU-3′ | 41 |
| | | 3′-UCCUUCAUUUCGAGAUGUUCU-5′ | 42 |
| siRNA | Luciferase siRNA | 5′-UACAUAACCGGACAUAAUCUU-3′ | 43 |
| | (neg control) | 3′-UUAUGUAUUGGCCUGUAUUAG-5′ | 44 |

### Example 7

### Evaluation of ASO Functional Uptake in the Presence of Vps28 Inhibitor

ASO and siRNAs

ASO 407988 was selected and evaluated for its functional uptake in MHT cells in the presence of Vps28 inhibitor.

ASO 407988 was prepared using the procedures published in the literature (Koller et al., Nucleic Acids Res., 2011, 39(11), 4795-47807) and the siRNAs were purchased from Dharmacon Research Inc. (Boulder, Colo., USA).

The ASO and siRNAs are described in Table 18. A subscript “s” between two nucleosides indicates a phosphorothioate internucleoside linkage (going 5′ to 3′ or 3′ to 5′). The absence of a subscript “s” between two nucleosides indicates a phosphodiester internucleoside linkage. Nucleosides without a subscript are ribonucleosides (RNA). Nucleosides with subscripts “d” are β-D-2′-deoxyribonucleosides. A subscript “e” indicates a 2′-O-methoxyethyl (MOE) modified nucleoside. ^{me}C indicates a 5-methyl cytosine nucleoside.

### Cell Culture and Transfection

MHT cells were cultured in MatTek glass-bottom dishes utilizing the method described in Example 1. To further characterize the uptake of ASO in the presence of Vsp28 inhibitor, cultured MHT cells were treated with Vsp28 siRNA-3 or neg control siRNA and ASO 407988. Luciferase siRNA was used as a negative control. Cells were plated at a density of 20,0000 cells per 35 mm dish and transfected using Opti-MEM containing 5 ug/mL Lipofectamine 2000. First transfection was performed using 40 nM concentration of Vsp28 siRNA-3 or negative control. After a treatment period of 4 hrs, transfection medium was replaced with complete growth medium and a second transfection was performed 24 hrs later in the same manner as above described above. 24 hrs later, an AF-488 conjugated ASO 407988 was added to complete growth medium (DMEM, 10% FBS) at 100 nM concentration. Cells were fixed with formaldehyde after 24 hrs and lysosomes were stained with Lamp1 antibody utilizing the method described below.

### Immunofluorescence

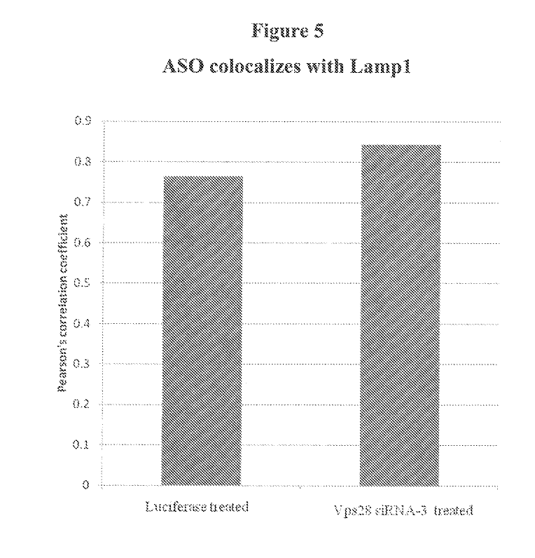

Cells were grown in glass-bottom dishes (MatTek). Cells were washed three times with 1×PBS, fixed at room temperature for 15 min with 4% formaldehyde and permeabilized for 5 min with 0.05% Saponin in 1×PBS. Cells were then incubated for 1 h with 1×PBS with 0.05% Saponin containing a rat anti-mouse LAMP1 antibody (1:1000, clone 1D4B, BD, Bioscience). After three washes (5 min each) with 1×PBS, cells were incubated for 1 h with PBS containing secondary antibodies against mouse. After three washes, slides were mounted with Dapi Fluoromount G (Southern Biotech). Cells were imaged with a confocal microscope (Olympus, Fluoview 1000) and images were processed using software FV10-ASW 2.1. Quantitative estimate of association (abundance) for proteins was measured by the Pearson's correlation coefficient utilizing the method described in the literature (Manders et al., J. Microsc., 1993, 169(3), 375-382. Results are presented in FIG. 5 and demonstrate that the bulk of ASO is localized in the lysosomes in both negative control siRNA treated cells and Vps28 siRNA-3 treated cells. The lysosomes in Vps28 siRNA-3 treated cells are enlarged and are reminiscent to multivesicular bodies.

TABLE 18
ASO and siRNAs

| RNA | Oligo No. | Composition | SEQ ID No. |
|---|---|---|---|
| ASO | 407988 | 5′-Gₑₛ^{m}CₑₛTₑₛTₑₛ^{m}CₑₛA_{ds}G_{ds}T_{ds}^{m}C_{ds}A_{ds} | 38 |
| | | T_{ds}G_{ds}A_{ds}^{m}C_{ds}T_{ds}Tₑₛ^{m}Cₑₛ^{m}CₑₛTₑₛTₑ-3′ | |
| siRNA | Vps28 siRNA-3 | 5′-GAAGUAAAGCUCUACAAGAUU-3′ | 41 |
| | | 3′-UCCUUCAUUUCGAGAUGUUCU-5′ | 42 |
| siRNA | Luciferase siRNA | 5′-UACAUAACCGGACAUAAUCUU-3′ | 43 |
| | (neg control) | 3′-UUAUGUAUUGGCCUGUAUUAG-5′ | 44 |

### Example 8

### Effect of Vps28 Depletion on Vesicle Size in MHT Cells

ASO and siRNA

ASO 407988 and Vps28 siRNA-3 from Table 18 were evaluated for the effect of Vps28 depletion on vesicle size in MHT cells.

### Cell Culture and Transfection

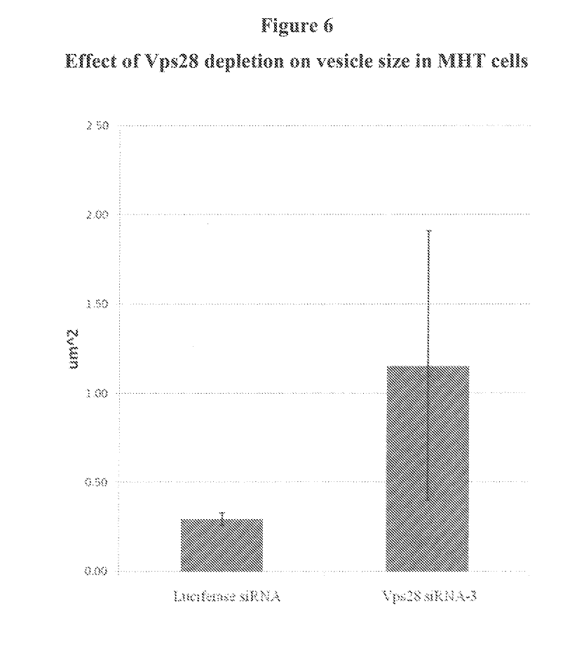

MHT cells were cultured utilizing the method described in Example 1. To evaluate the effect of Vps28 depletion on vesicle size in the presence of Vps28 inhibitor, cultured MHT cells were treated with Vps28 siRNA-3 and negative control siRNA. Luciferase siRNA was used as a negative control. Cells were plated at a density of 20,0000 cells per 35 mm glass bottom dish (MatTek) and transfected using Opti-MEM containing 5 ug/mL Lipofectamine 2000 at 40 nM concentration of Vps28 siRNA-3 or luciferase siRNA. After a treatment period of 4 hrs, transfection medium was replaced with complete growth medium and a second transfection was performed 24 hrs later in the same manner as described above. 24 hrs later, an AF-488 conjugated ASO 407988, was added to complete growth medium (DMEM, 10% FBS) at 100 nM concentration. Cells were fixed with formaldehyde after 24 hrs and ASO containing vesicle size was measured with Fluoview1000. Lysosomes were stained with Lamp1 antibody and Vsp28 was visualized with Vsp28 antibody utilizing the method described in Example 7. Results are presented in FIG. 6 and demonstrate that Vps28 depletion results in an increase in vesicle size compared to negative control.

### Example 9

### Evaluation of ASOs Functional Uptake in Vps28 Depleted Cells

ASO and siRNAs

ASO 407988 from Table 18 was evaluated for its functional uptake in Vps28 depleted MHT cells.

### Cell Culture and Transfection

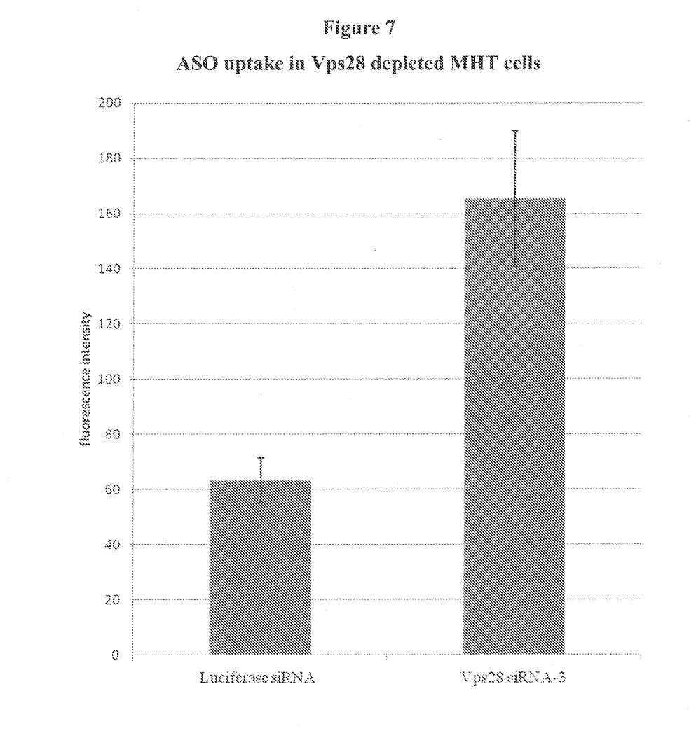

MHT cells were isolated and cultured utilizing the method described in Example 1. To characterize the uptake of ASO in the presence of Vsp28 inhibitor, cultured MHT cells were treated with Vsp28 siRNA-3 or neg control siRNA and ASO 407988. Cells were plated at a density of 200,000 cells per 35 mm glass bottom dish (MatTek) and transfected using Opti-MEM containing 5 ug/mL Lipofectamine 2000. First transfection was performed using 40 nM concentration of Vsp28 siRNA-3 or negative control siRNA. Luciferase siRNA was used as a negative control. After a treatment period of 4 hrs, transfection medium was replaced with complete growth medium and a second transfection was performed 24 hrs later in the same manner as described above. 24 hrs later, an AF-488 conjugated ASO 407988 was added to complete growth medium (DMEM, 10% FBS) at 100 nM concentration. Cells were fixed with formaldehyde after 24 hrs and fluorescence intensity was measured with FV1000 (Olympus) utilizing the method described in Example 7. Results are presented in FIG. 7 and demonstrate that ASO uptake into MHT cells is increased in Vps28 siRNA-3 treated cells as compared to negative control.

### Example 10

### Certain Methods

### Cells and Reagents

DMEM supplemented with 10% fetal calf serum trypsin, Penicillin, Streptomycin and Lipofectamine2000 were purchased from Invitrogen (Carlsbad, Calif.). MHT cells (Mouse Hepatocellular carcinoma cell line) were isolated as described previously (Koller et al., Nucleic Acids Res., 2011, 39(11), 4795-4807). MHT cells were cultured in DMEM supplemented with 10% fetal calf serum, streptomycin (0.1 ug/ml), and penicillin (100 units/ml). siRNA treatment was performed using Opti-MEM (Invitrogen) containing 5 μg/ml Lipofectamine 2000 at the indicated amount of siRNA for 4 h at 37° C., as described previously (Dean et al., J. Biol. Chem., 1994, 269(23), 16416-16424; and Antisense Nucleic Acid Drug Dev., 1997, 7(3), 229-233).

Preparation of Synthetic siRNA and siRNA Transfection

Synthetic unmodified siRNAs were purchased from Thermo Scientific, (Boulder, Colo.) and Life Technologies (Carlsbad, Calif.). siRNA duplexes were formed according to the manufacturer's instructions and as previously reported (Koller et al., Nucleic Acids Res., 2011, 39(11), 4795-4807).

### Taqman RT-PCR

Total RNA was harvested at 16-24 hours post-transfection using an RNeasy 3000 BioRobot (Qiagen, Valencia, Calif.). Reduction of target mRNA expression was determined by real time RT-PCR using StepOne (Applied Biosystems, Foster City, Calif.). The sequences for the primer/probe set used in the RT-PCR reaction are listed Table 19, below.

TABLE 19
Primers and Probes

| | | | | SEQ ID |
|---|---|---|---|---|
| Target | Forward (5′ to 3′) | Reverse (5′ to 3′) | Probe (5′ to 3′) | Nos. |
| SR-B1 | TGACAACGACACC | ATGCGACTTGTCA | CGTGGAGAACCGCAGC | 26, 27, 28 |
| | GTGTCCT | GGCTGG | CTCCATT | |
| Vps28 | CTTCGATCTGGAGT | TTCCTGTCTCGGT | CGTTGGCACGACACCT | 35, 36, 37 |
| | CCGCTTA | GAGGCTTA | TCAGGGACT | |

### Western Blotting

siRNA treated cells were lysed in RIPA lysis buffer containing 1% Triton X-100, 0.1% SDS, 0.25% Sodium deoxycholate, 150 mM NaCl, Tris pH 7.5 and complete protease inhibitor mix with EDTA (Roche, Indianapolis, Ind.). Equal amounts of protein were resolved on a SDS-PAGE gel and transferred to Nitrocellulose membranes. The membranes were blocked for 1 h with blocking buffer (Li-COR, Lincoln, Nebr.) containing 0.1% Tween-20. Proteins were detected using LDLR antibody AF2148 (R&D, Minneapolis, Minn.) or Vps28 antibody NBP1-03506 (Novus Biologicals, Littleton, Colo.). After incubation with dye-conjugated secondary antibodies, blots were visualized using Odyssey (Li-COR, Lincoln, Nebr.).

### Flow Cytometry

A fluorescein-conjugated SSO was added to MHT cells for 24 hrs. Cells were trypsinized and analyzed on FacsCalibur. BODIPY® FL conjugated LDL and acetylated, Alexa Fluor® 488 conjugated and acetylated LDL-(50 μg/ml) was added to cells, respectively. 4 hrs later cells were trypsinized and uptake of LDL was measured using the FacsCalibur.

### Example 11

### Effect of Vps28 Depletion on Uptake of Acetylated LDL or LDL and Protein Levels of LDL Receptor (LDLR) in the Presence of Vps28 Inhibitor

The effect of Vps28 depletion on uptake of acetylated LDL or LDL and protein levels of LDL receptor (LDLR) in MHT cells in the presence of Vps28 inhibitor was evaluated.

Vps28 modulator was tested. As shown in the table below, Vps28 was an siRNA targeted to Vps28 and was purchased from Dharmacon Research Inc. (Boulder, Colo., USA).

The siRNAs are described in Table 20, wherein the internucleoside linkages are phosphodiesters and the nucleosides are ribonucleosides (RNAs).

### Cell Culture, Transfection and Analysis

MHT cells were isolated from a hepatocellular carcinoma tumor which developed in transgenic mouse expressing SV40 large T-antigen under the CRP promoter (Ruther et al., Oncogene, 1993, 8, 87-93) and cultured in DMEM supplemented with 10% fetal bovine serum (FBS), streptomycin (0.1 ug/mL), and penicillin (100 U/mL).

To evaluate the effect of Vps28 depletion on uptake of acetylated LDL or LDL and protein levels of LDLR in the presence of Vps28 inhibitor, cultured MHT cells were transfected with Vps28 siRNA and luciferase siRNA, which was used as a negative control. Cells were plated at a density of 200,000 cells per 6-well and transfected using Opti-MEM containing 5 μg/mL Lipofectamine 2000 with 40 nM or 50 nM concentration of siRNA. After a treatment period of 72 hrs, LDLR protein levels were measured by western blot and uptake of acetylated LDL or LDL was measured with flow cytometry using the methods described in Example 1. Mean results from three replicates are presented below.

As illustrated in Tables 20 and 20a, depletion of Vps28 results in an increased in LDLR protein levels and an increase in LDL-uptake while uptake of acetylated LDL was lowered compared to the control

TABLE 20
Effect of Vps28 depletion on LDLR protein levels and LDL-uptake

| | | Conc. | LDLR | LDL-uptake | SEQ ID |
|---|---|---|---|---|---|
| Oligo No. | Composition | (nM) | protein level | level | No. |
| Vps28 | 5′-UCGGAAGGCAGCUUUGUACUU-3′ | 40 | 238 | 79 | 39 |
| siRNA | 3′-UUAGCCUUCCGUCGAAACAUG-5′ | | | | 40 |
| Luciferase | 5′-UACAUAACCGGACAUAAUCUU-3′ | 40 | 100 | 61 | 43 |
| siRNA | 3′-UUAUGUAUUGGCCUGUAUUAG-5′ | | | | 44 |
| (neg control) | | | | | |

TABLE 20a
Effect of Vps28 depletion on Acetylated LDL-uptake

| | Conc. | Acetylated | |
|---|---|---|---|
| Oligo No. | (nM) | LDL-uptake level | SEQ ID No. |
| Vps28 | 50 | 129.49 | 39-40 |
| siRNA | | | |
| Luciferase | 50 | 225.17 | 43-44 |
| siRNA | | | |
| (neg control) | | | |

### Example 12

### Effect of Single-Stranded Antisense Oligonucleotide (SSO) on SRB-1 mRNA Levels in the Presence of LDLR Inhibitor

The SSO 353382 was evaluated for its effect on SRB-1 mRNA levels in MHT cells in the presence of LDLR inhibitor. LDLR is a key regulator of cellular LDL uptake and plasma cholesterol levels.

LDLR modulator was tested. As shown in the table below, LDLR was a pool of four siRNAs targeted to LDLR and are denoted as “LDLR siRNA-1,” “LDLR siRNA-2,” “LDLR siRNA-3,” “LDLR siRNA-3,” and “LDLR siRNA-4.” These were purchased from Dharmacon Research Inc. (Boulder, Colo., USA).

The SSO 353382 is a 5-10-5 MOE gapmer, wherein the internucleoside linkages are phosphorothioates and was prepared using the procedures published in the literature (Koller et al., Nucleic Acids Res., 2011, 39(11), 4795-4807).

The sequences for the SSO and siRNAs are described in Table 21. A subscript “s” between two nucleosides indicates a phosphorothioate internucleoside linkage (going 5′ to 3′ or 3′ to 5′). The absence of a subscript “s” between two nucleosides indicates a phosphodiester internucleoside linkage. Nucleosides without a subscript are ribonucleosides (RNA). Nucleosides with subscripts “d” are β-D-2′-deoxyribonucleosides. A subscript “e” indicates a 2′-O-methoxyethyl (MOE) modified nucleoside. ^{me}C indicates a 5-methyl cytosine nucleoside.

### Cell Culture, Transfection and Analysis

MHT cells were isolated from a hepatocellular carcinoma tumor which developed in transgenic mouse expressing SV40 large T-antigen under the CRP promoter (Ruther et al., Oncogene, 1993, 8, 87-93) and cultured in DMEM supplemented with 10% fetal bovine serum (FBS), streptomycin (0.1 ug/mL), and penicillin (100 U/mL).

To evaluate the effect of SSO on SRB-1 mRNA levels in the presence of LDLR inhibitor, cultured MHT cells were transfected with luciferase siRNA (negative control) and LDLR siRNA. LDLR siRNA comprises a mixture of four siRNAs as shown in the table, below. Cells were plated at a density of 7,500 cells per well and transfected using Opti-MEM containing 5 μg/mL Lipofectamine 2000. First transfection was performed using 75 nM concentration of luciferase or LDLR siRNA. After a treatment period of 4 hrs, transfection medium was replaced with complete growth medium. 48 hrs later, SSO 353382 was added to complete growth medium (DMEM, 10% FBS) at concentrations listed in the table below. RNA was isolated from cells after 24 hours and SRB-1 mRNA levels were measured by qRT-PCR as described in Example 1. The expression data was normalized to RIBOGREEN (Invitrogen) and the mean results from three replicates are presented in Table 22, below.

As illustrated, a decrease in SSO potency was observed in MHT cells when LDLR was depleted as compared to the control. As expected, treatment with LDLR inhibitor resulted in a 35% reduction in LDLR mRNA levels in MHT (Table 23).

TABLE 21
SSO targeting SRB-1 and LDLR siRNA

| | | | SEQ |
|---|---|---|---|
| | | | ID |
| RNA | ISIS No. | Composition | No. |
| SSO | 353382 | 5′-Gₑₛ^{m}CₑₛTₑₛTₑₛ^{m}CₑₛA_{ds}G_{ds}T_{ds}^{m}C_{ds}A_{ds} | 38 |
| | | T_{ds}G_{ds}A_{ds}^{m}C_{ds}T_{ds}Tₑₛ^{m}Cₑₛ^{m}CₑₛTₑₛTₑ-3′ | |
| LDLR | 423059 | 5′-ACACGGAGGUGACCAACAATT-3′ | 55 |
| siRNA-1 | 423299 | 5′-UUGUUGGUCACCUCCGUGUUU-3′ | 56 |
| LDLR | 423060 | 5′-GAAAAUGCAUCGCUAGCAATT-3′ | 57 |
| siRNA-2 | 423300 | 5′-UUGCUAGCGAUGCAUUUUCTT-3′ | 58 |
| LDLR | 423061 | 5′-GCAAAUCAUCCAUAUGCAUTT-3′ | 59 |
| siRNA-3 | 423301 | 5′-AUGCAUAUGGAUGAUUUGCTT-3′ | 60 |
| LDLR | 423062 | 5′-GGCCGUCUCUAUUGGGUUGTT-3′ | 61 |
| siRNA-4 | 423302 | 5′-CAACCCAAUAGAGACGGCCTT-3′ | 62 |

TABLE 22
Effect of SSO on SR-B1 mRNA level
in the presence of LDLR inhibitor

| siRNA + SSO | Conc. of | SRB-1 mRNA level | |
|---|---|---|---|
| Treatment | ASO (nM) | (% control) | SEQ ID No. |
| LDLR siRNA + | 10000 | 47 | 55-62; 38 |
| SSO 353382 | 2000 | 59 | |
| | 400 | 75 | |
| | 80 | 82 | |
| | 16 | 86 | |
| | 3.2 | 102 | |
| | 0.64 | 112 | |
| | 0.16 | 121 | |
| Luciferase siRNA | 10000 | 30 | 43-44; 38 |
| (neg control) + | 2000 | 36 | |
| SSO 353382 | 400 | 42 | |
| | 80 | 55 | |
| | 16 | 68 | |
| | 3.2 | 72 | |
| | 0.64 | 79 | |
| | 0.16 | 95 | |

TABLE 23
LDLR mRNA levels in the presence
of LDLR inhibitor in MHT cells

| | LDLR mRNA level | |
|---|---|---|
| Oligo No. | (% control) | SEQ ID No. |
| LDLR siRNA 1-4 | 65 | 55-62 |
| Luciferase siRNA | 100 | 43, 44 |
| (neg control) | | |

Previously, we have shown that inhibition of Vps28, a member of the ESCRT family, sensitizes cells to target reduction of a single stranded antisense oligonucleotides. We now show that inhibition of Vps28 results in an increase in LDLR expression and LDL-uptake. When LDLR expression is inhibited, potency of the SSO decreases. This result suggests that LDLR plays a role in productive SSO uptake. Thus, increasing LDLR expression can be used as a method to increase potency of SSO.

### Example 13

### Effect of SSO on SR-B1 mRNA Levels in the Presence of Vps28 Inhibitor in MEFs

SSO 353382 was selected and evaluated for its effect on SR-B1 mRNA levels in MEFs (Mouse Embryonic Fibroblasts) in the presence and absence of Vps28 inhibitor. The SSO 353382, Vps28 siRNA-1 and negative control siRNA were previously described in Table 3.

Day 10.5 embryos were dissected and diced in trypsin. Mouse embryo fibroblasts (MEFs) were cultured on collagen-coated plates in DMEM+10% FBS. Cells were plated at a density of 7,500 cells per 96-well and transfected using Opti-MEM containing 5 μg/mL Lipofectamine 2000. First transfection was performed using 50 nM concentration of Vps28 siRNA-1 or negative control siRNA. After a treatment period of 4 hrs, transfection medium was replaced with complete growth medium and a second transfection was performed 24 hrs later in the same manner as above. 24 hrs later, SSO 353382 targeting SR-B1 was added to complete growth medium (DMEM, 10% FBS) at concentrations listed in Table 24, below. RNA was isolated from cells after 24 hours and SR-B1 mRNA levels were measured by qRT-PCR as described previously. The expression data was normalized to RIBOGREEN (Invitrogen) and mean values of three replicates are provided below.

As illustrated in Table 24, an increase in reduction of SR-B1 mRNA levels was observed in MEFs for SSO 353382 in the presence of Vps28 inhibitor as compared to the negative control. The results demonstrate that inhibition of Vps28 increases the potency of SSO 353382. As expected, treatment with Vps28 siRNA reduced Vps28 mRNA levels in MEFs.

TABLE 24
Inhibition of SR-B1 mRNA level with SSO in
the presence of Vps28 inhibitor in MEFs

| | Conc. of | SR-B1 mRNA level |
|---|---|---|
| Treatment | SSO (nM) | (% control) |
| Vps28 siRNA-1 + | 10000 | 13 |
| SSO 353382 | 2000 | 17 |
| | 400 | 23 |
| | 80 | 53 |
| | 16 | 72 |
| | 3 | 115 |
| | 1 | 117 |
| | 0 | 109 |
| Neg. con. siRNA + | 10000 | 30 |
| SSO 353382 | 2000 | 44 |
| | 400 | 62 |
| | 80 | 77 |
| | 16 | 96 |
| | 3 | 95 |
| | 1 | 98 |
| | 0 | 98 |

### Example 14

### Effect of SSO on SR-B1 mRNA Levels in the Presence of Vps28 Inhibitor in Primary Mouse Hepatocytes

SSO 353382 was selected and evaluated for its effect on SR-B1 mRNA levels in the presence and absence of Vps28 inhibitor. The SSO 353382 and Vps28 siRNA-1 were previously described in Table 3.

Primary mouse hepatocytes were isolated from Balb/C mice and cultured on collagen-coated plates in DMEM with 10% FBS. Cells were plated at a density of 7,500 cells per 96-well and transfected using Opti-MEM containing 5 μg/mL Lipofectamine 2000. First transfection was performed using 75 nM concentration of Vps28 siRNA-1 or negative control siRNA. After a treatment period of 4 hrs, transfection medium was replaced with complete growth medium and SSO 353382 targeting SR-B1 was added 2, 3 and 6 days later at concentrations listed in Table 25, below. RNA was isolated from cells after 24 hours and SR-B1 mRNA levels were measured by qRT-PCR as described previously. The expression data was normalized to RIBOGREEN (Invitrogen) and mean values of three replicates are provided below.

As illustrated in Table 25, inhibition of Vps28 increases the potency of SSO 353382 targeting SR-B1 in primary mouse hepatocytes 2, 3 and 6 days after Vps28 siRNA transfection compared to the negative control.

TABLE 25
Effect of SSO on SR-B1 mRNA levels in primary mouse hepatocytes
2, 3 and 6 days after Vps28 siRNA transfection

| | | Conc. of | SR-B1 mRNA level |
|---|---|---|---|
| Treatment | Days | SSO (nM) | (% control) |
| Vps28 siRNA-1 + | 2 | 12800 | 14.78 |
| SSO 353382 | | 3200 | 21.49 |
| | | 800 | 29.32 |
| | | 200 | 32.26 |
| | | 50 | 47.95 |
| | | 12.5 | 66.46 |
| | | 3.125 | 74.68 |
| | | 0.78 | 81.70 |
| | 3 | 12800 | 5.26 |
| | | 3200 | 8.23 |
| | | 800 | 11.00 |
| | | 200 | 12.23 |
| | | 50 | 19.95 |
| | | 12.5 | 53.37 |
| | | 3.125 | 61.10 |
| | | 0.78 | 58.62 |
| | 6 | 12800 | 4.43 |
| | | 3200 | 4.49 |
| | | 800 | 7.00 |
| | | 200 | 9.80 |
| | | 50 | 24.14 |
| | | 12.5 | 34.33 |
| | | 3.125 | 29.99 |
| | | 0.78 | 22.42 |
| Neg. con. siRNA + | 2 | 12800 | 32.00 |
| SSO 353382 | | 3200 | 43.54 |
| | | 800 | 51.65 |
| | | 200 | 57.59 |
| | | 50 | 80.81 |
| | | 12.5 | 76.85 |
| | | 3.125 | 82.90 |
| | | 0.78 | 85.98 |
| | 3 | 12800 | 12.92 |
| | | 3200 | 26.04 |
| | | 800 | 46.23 |
| | | 200 | 58.00 |
| | | 50 | 56.09 |
| | | 12.5 | 59.43 |
| | | 3.125 | 97.99 |
| | | 0.78 | 100.05 |
| | 6 | 12800 | 29.15 |
| | | 3200 | 24.66 |
| | | 800 | 37.00 |
| | | 200 | 42.02 |
| | | 50 | 68.73 |
| | | 12.5 | 78.69 |
| | | 3.125 | 69.97 |
| | | 0.78 | 65.26 |

### Example 15

### Effects of SSOs Comprising Constrained Ethyl (i.e. cEt) or Fluoro-HNA Modifications on SR-B1 mRNA Levels in the Presence of Vps28 Inhibitor

The SSOs comprising cEt or fluoro-HNA modifications were selected and tested for their effects on SR-B1 mRNA levels in the presence and absence of Vps28 inhibitor.

The SSO 479781 and 479782 were prepared using similar procedures reported in the literature (Egli et al., J. Am. Chem. Soc., 2011, 133(41), 16642-16649; and Pallan, et al., Chem. Com. (Camb), 2012, 48(66), 8195-8197) and are described in Table 26, below. Subscripts “s” indicate phosphorothioate internucleoside linkages. Subscripts “k” indicate constrained ethyl bicyclic nucleosides (i.e. cEt). Subscripts “g” indicate F-HNA modified nucleosides. Subscripts “d” indicate β-D-2′-deoxyribonucleosides. “^{m}C” indicates 5-methylcytosine nucleoside.

Vps28 siRNA-1 and negative control siRNA were purchased from Ambion, Life Technologies. (Carlsbad, Calif., USA) and were described previously in Table 3.

MHT cells were isolated and cultured according to the methods described previously. Cells were plated at a density of 7,500 cells per 96-well and transfected using Opti-MEM containing 5 μg/mL Lipofectamine 2000. First transfection was performed using 75 nM concentration of Vps28 siRNA-1 or negative control siRNA. After a treatment period of 4 hrs, transfection medium was replaced with complete growth medium and a second transfection was performed 24 hrs later in the same manner as above. 24 hrs later, SSO 353382 targeting SR-B1 was added to complete growth medium (DMEM, 10% FBS) at concentrations listed in Table 26, below. RNA was isolated from cells after 24 hours and SR-B1 mRNA levels were measured by qRT-PCR as described previously. The expression data was normalized to RIBOGREEN (Invitrogen) and mean values of three replicates are provided in Table 27, below.

As illustrated in Table 27, an increase in reduction of SR-B1 mRNA levels was observed in MHT cells for SSO 479781 and 479782 in the presence of Vps28 inhibitor as compared to the negative control. The results demonstrate that inhibition of Vps28 increases the potency of SSOs.

TABLE 26
SSOs targeting SR-B1 and siRNAs targeting Vps28 in MHT cells

| | | 5′ and 3′ wing | Gap | SEQ ID |
|---|---|---|---|---|
| SSO | Composition (5′ to 3′) | chemistry | chemistry | No. |
| 479781 | TₖₛT_{ds}^{m}CₖₛA_{ds}G_{ds}T_{ds}^{m}C_{ds}A_{ds} | cEt/deoxy | Full deoxy | 63 |
| | T_{ds}G_{ds}A_{ds}^{m}C_{ds}T_{ds}Tₖₛ^{m}C_{ds}^{m}Cₖ | | | |
| 479782 | G_{gs}^{m}C_{ds}T_{gs}T_{ds}^{m}C_{gs}A_{ds}G_{ds}T_{ds}^{m}C_{ds}A_{ds} | F-HNA/deoxy | Full deoxy | 38 |
| | T_{ds}G_{ds}A_{ds}^{m}C_{ds}T_{ds}T_{gs}^{m}C_{ds}^{m}C_{gs}T_{ds}T_{g} | | | |

TABLE 27
Effect of SSOs comprising cEt or F-HNA modifications on
SR-B1 mRNA levels in the presence of Vps28 inhibitor

| | 5′ and 3′ | | | SR-B1 |
|---|---|---|---|---|
| | wing | Gap | Conc. of | mRNA level |
| Treatment | chemistry | chemistry | SSO (nM) | (% control) |
| Vps28 | cEt/deoxy | Full deoxy | 10000 | 21.23 |
| siRNA-1 + | | | 2000 | 20.17 |
| SSO 479781 | | | 400 | 25.17 |
| | | | 80 | 29.66 |
| | | | 16 | 69.89 |
| | | | 3.20 | 112.94 |
| | | | 0.64 | 121.79 |
| | | | 0.13 | 128.51 |
| Neg. con. | cEt/deoxy | Full deoxy | 10000 | 44.15 |
| siRNA + | | | 2000 | 58.21 |
| SSO 479781 | | | 400 | 72.65 |
| | | | 80 | 78.91 |
| | | | 16 | 110.72 |
| | | | 3.20 | 116.30 |
| | | | 0.64 | 115.51 |
| | | | 0.13 | 109.25 |
| Vps28 | F-HNA/deoxy | Full deoxy | 10000 | 17.89 |
| siRNA-1 + | | | 2000 | 17.43 |
| SSO 479782 | | | 400 | 15.90 |
| | | | 80 | 18.75 |
| | | | 16 | 31.36 |
| | | | 3.20 | 77.34 |
| | | | 0.64 | 118.62 |
| | | | 0.13 | 124.23 |
| Neg. con. | F-HNA/deoxy | Full deoxy | 10000 | 37.75 |
| siRNA + | | | 2000 | 67.96 |
| SSO 479782 | | | 400 | 78.53 |
| | | | 80 | 85.63 |
| | | | 16 | 86.80 |
| | | | 3.20 | 106.49 |
| | | | 0.64 | 103.24 |
| | | | 0.13 | 116.02 |

### Example 16

### Effect of SSOs on SR-B1 mRNA Levels in Vps28 Depleted Cells

The effect of SSOs on SR-B1 mRNA levels in Vps28 depleted cells was evaluated. The SSOs and siRNA are described in Table 28, below. The SSO 353382 and Vps28 siRNA-3 were previously described in Table 3.

The control SSO (141923) and Vps28 SSO (524385) are 5-10-5 MOE gapmers and are described in Table 28, below. Subscripts “s” indicate phosphorothioate internucleoside linkages. Subscripts “e” indicates 2′-O-methoxyethyl (MOE) modified nucleosides. Subscripts “d” indicate β-D-2′-deoxyribonucleosides. “^{m}C” indicates 5-methylcytosine nucleoside.

MHT cells were isolated and cultured according to the methods described previously. Cells were plated at a density of 7,500 cells per 96-well and transfected using Opti-MEM containing 5 μg/mL Lipofectamine 2000. First transfection was performed using 75 nM concentration of Vps28 siRNA-3, Vps28 SSO (524385), control SSO (141923) or untreated control (UTC). After a treatment period of 4 hrs, transfection medium was replaced with complete growth medium and a second transfection was performed 24 hrs later in the same manner as above. 24 hrs later, SSO 353382 targeting SR-B1 was added to complete growth medium (DMEM, 10% FBS) at concentrations listed in Table 29, below. RNA was isolated from cells after 24 hours and SR-B1 mRNA levels were measured by qRT-PCR as described previously. The expression data was normalized to RIBOGREEN (Invitrogen) and mean values of three replicates are provided in Table 29, below.

As illustrated, an increase in reduction of SR-B1 mRNA levels was observed in MHT cells for SSO 353382 in the presence of Vps28 inhibitors compared to untreated control. The results demonstrate that inhibition of Vps28 with siRNA (Vps28 siRNA-3) or SSO (524385) increases the potency of SSO 353382.

TABLE 28
SSOs targeting SR-B1 in the presence of Vps28 inhibitors (SSO or siRNA)

| RNA | Oligo No. | Composition (5′ to 3′) | Chemistry | SEQ ID No. |
|---|---|---|---|---|
| Ctrl SSO | 141923 | ^{m}Cₑₛ^{m}CₑₛTₑₛTₑₛ^{m}Cₑₛ^{m}C_{ds}^{m}C_{ds}T_{ds}G_{ds}A_{ds} | 5-10-5 | 64 |
| | | A_{ds}G_{ds}G_{ds}T_{ds}T_{ds}^{m}Cₑₛ^{m}CₑₛTₑₛ^{m}Cₑₛ^{m}Cₑ | MOE-DNA-MOE | |
| | | | gapmer | |
| Vps28 SSO | 524385 | AₑₛTₑₛ^{m}Cₑₛ^{m}CₑₛTₑₛG_{ds}A_{ds}^{m}C_{ds}A_{ds}A_{ds} | 5-10-5 | 65 |
| | | T_{ds}A_{ds}T_{ds}A_{ds}G_{ds}Gₑₛ^{m}CₑₛAₑₛTₑₛ^{m}Cₑ | MOE-DNA-MOE | |
| | | | gapmer | |

TABLE 29
Effect of SSO on SR-B1 mRNA level
in the presence of Vps28 inhibitors

| | Conc. of | SRB-1 mRNA level |
|---|---|---|
| Treatment | ASO (nM) | (% control) |
| UTC + | 10000 | 41.10 |
| SSO 353382 | 2000 | 51.39 |
| | 400 | 59.02 |
| | 80 | 63.98 |
| | 16 | 83.51 |
| | 3.2 | 96.85 |
| | 0.64 | 103.80 |
| | 0.16 | 105.75 |
| ISIS 141923 | 10000 | 32.61 |
| (Ctrl SSO) + | 2000 | 46.92 |
| SSO 353382 | 400 | 54.05 |
| | 80 | 56.88 |
| | 16 | 73.31 |
| | 3.2 | 81.32 |
| | 0.64 | 90.25 |
| | 0.16 | 97.05 |
| Vps28 siRNA-3 + | 10000 | 20.52 |
| SSO 353382 | 2000 | 27.47 |
| | 400 | 33.66 |
| | 80 | 46.06 |
| | 16 | 69.45 |
| | 3.2 | 105.06 |
| | 0.64 | 121.58 |
| | 0.16 | 120.17 |
| SSO 524385 | 10000 | 15.24 |
| (Vps28 SSO) + | 2000 | 20.88 |
| SSO 353382 | 400 | 33.35 |
| | 80 | 48.75 |
| | 16 | 73.43 |
| | 3.2 | 84.62 |
| | 0.64 | 99.51 |
| | 0.16 | 112.93 |

### Example 17

### Effect of SSO on SR-B1 mRNA Levels in the Presence of Hrs Inhibitor

SSO 353382 was selected and evaluated for its effect on SR-B1 mRNA levels in MHT cells and b.END cells in the presence and absence of Hrs inhibitor. Hrs (hepatocyte growth factor-regulated tyrosine kinase substrate) is a member of the ESCRT-0 complex. The SSO 353382 and negative control siRNA were described previously in Table 3.

The Hrs siRNA was purchased from Dharmacon Research Inc. (Boulder, Colo., USA) and is described in Table 30, below. The nucleosides are ribonucleosides (RNA) and the internucleoside linkages are phosphodiesters.

MHT cells and b.END cells were cultured in the same manner as described in Example 1. Cultured MHT cells and b.END cells were treated with Hrs or negative control siRNA. Cells were plated at a density of 7,500 cells per well and transfected using Opti-MEM containing 5 μg/mL Lipofectamine 2000. First transfection was performed using 40 nM concentration of Hrs or negative control siRNA. These siRNAs are denoted as “Hrs siRNA” for Hrs inhibitor and “Ctrl siRNA” for negative control. After a treatment period of 4 hours, transfection medium was replaced with complete growth medium and a second transfection was performed 24 hrs later in the same manner as described above. 24 hrs later, SSO 353382 targeting SR-B1 was added to complete medium at concentrations listed in Table 31. RNA was isolated from cells after 24 hours and target mRNA levels were measured by qRT-PCR utilizing the method described in Example 1. The expression data was normalized to RIBOGREEN (Invitrogen) and mean values of three replicates are provided below.

As illustrated in Table 31, an increase in reduction of SR-B1 mRNA levels was observed in MHT and b.END cells for SSO 353382 in the presence of Hrs inhibitor as compared to the negative control. The results demonstrate that inhibition of Hrs increases the potency of SSO 353382. As expected, treatment with Hrs inhibitor reduced Hrs mRNA levels in MHT and b.END cells.

TABLE 30
Hrs siRNA

| Oligo No. | Composition | SEQ ID No. |
|---|---|---|
| Hrs siRNA | 5′-GGACAAGCUGGCACAGAUATT-3′ | 66 |
| | 5′-UAUCUGUGCCAGCUUGUCCTT-3′ | 67 |

TABLE 31
Effect of SSO on SR-B1 mRNA levels in the presence
of Hrs inhibitor in MHT cells and b.END cells

| | |
|---|---|
| | SR-B1 mRNA level |
| Conc. of | (% control) |

| Treatment | SSO (nM) | MHT cells | b.END cells |
|---|---|---|---|
| Hrs siRNA + | 10000 | 25.69 | 21.00 |
| SSO 353382 | 2000 | 31.02 | 23.14 |
| | 400 | 33.07 | 29.71 |
| | 80 | 52.64 | 38.85 |
| | 16 | 85.16 | 53.21 |
| | 3.2 | 97.19 | 71.62 |
| | 0.64 | 83.81 | 89.62 |
| | 0.16 | 133.54 | 93.25 |
| Ctrl siRNA | 10000 | 25.71 | 23.16 |
| (neg control) + | 2000 | 41.58 | 31.30 |
| SSO 353382 | 400 | 54.91 | 43.37 |
| | 80 | 60.86 | 50.86 |
| | 16 | 85.08 | 76.12 |
| | 3.2 | 114.57 | 103.62 |
| | 0.64 | 112.28 | 79.14 |
| | 0.16 | 115.55 | 125.10 |

### Example 18

### Effect of SSO on SR-B1 mRNA Levels in the Presence of Mvb12a Inhibitor

SSO 353382 was selected and evaluated for its effect on SR-B1 mRNA levels in MHT cells and b.END cells in the presence and absence of Mvb12a inhibitors. Mvb12a is another member of the ESCRT pathway that may be involved in the functional uptake of SSOs. The SSO 353382 and negative control siRNA were described previously in Table 3.

The Mvb12a siRNAs were purchased from Ambion, Life Technologies (Carlsbad, Calif., USA) and are described in Table 32, below. The nucleosides are ribonucleosides (RNA) and the internucleoside linkages are phosphodiesters.

MHT cells and b.END cells were cultured in the same manner as described in Example 1. Cultured MHT cells and b.END cells were treated with two different Mvb12a siRNAs or negative control siRNA targeting SR-B1. Cells were plated at a density of 7,500 cells per well and transfected using Opti-MEM containing 5 μg/mL Lipofectamine 2000. First transfection was performed using 40 nM concentration of Mvb12a siRNA or negative control siRNA. These siRNAs are denoted as “Mvb12a siRNA-1” or “Mvb12a siRNA-2” for Mvb12a inhibitors and “Ctrl siRNA” for negative control. After a treatment period of 4 hours, transfection medium was replaced with complete growth medium and a second transfection was performed 24 hrs later in the same manner as described above. 24 hrs later, SSO 353382 targeting SR-B1 was added to complete medium at concentrations listed in Table 33. RNA was isolated from cells after 24 hours and target mRNA levels were measured by qRT-PCR utilizing the method described in Example 1. The expression data was normalized to RIBOGREEN (Invitrogen) and mean values of three replicates are provided below.

As illustrated in Table 33, an increase in reduction of SR-B1 mRNA levels was observed in MHT and b.END cells for SSO 353382 in the presence of Mvb12a inhibitors as compared to the negative control. The results demonstrate that inhibition of Mvb12a increases the potency of SSO 353382. As expected, treatment with Mvb12a inhibitors reduced Mvb12a mRNA levels in MHT and b.END cells.

TABLE 32
Mvb12a siRNAs

| | | SEQ ID |
|---|---|---|
| Oligo No. | Composition | No. |
| Mvb12a siRNA-1 | 5′-CCUGACGAUCAAAUCACUGTT-3′ | 68 |
| | 5′-CAGUGAUUUGAUCGUCAGGTC-3′ | 69 |
| Mvb12a siRNA-2 | 5′-GGAGUAUAACUAUGGCUUCTT-3′ | 70 |
| | 5′-GAAGCCAUAGUUAUACUCCTT-3′ | 71 |

TABLE 33
Effect of SSO on SR-B1 mRNA levels in the presence
of Mvb12a inhibitors in MHT cells and b.END cells

| | |
|---|---|
| | SR-B1 mRNA level |
| Conc. of | (% control) |

| Treatment | SSO (nM) | MHT cells | b.END cells |
|---|---|---|---|
| Mvb12a siRNA-1 + | 10000 | 21 | 18 |
| SSO 353382 | 2000 | 32 | 28 |
| | 400 | 38 | 33 |
| | 80 | 42 | 37 |
| | 16 | 72 | 48 |
| | 3.2 | 86 | 78 |
| | 0.64 | 96 | 96 |
| | 0.16 | 88 | 99 |
| Mvb12a siRNA-2 + | 10000 | 14 | 25 |
| SSO 353382 | 2000 | 25 | 35 |
| | 400 | 29 | 47 |
| | 80 | 31 | 49 |
| | 16 | 55 | 53 |
| | 3.2 | 104 | 82 |
| | 0.64 | 104 | 97 |
| | 0.16 | 109 | 99 |
| Ctrl siRNA | 10000 | 26 | 41 |
| (neg control) + | 2000 | 42 | 53 |
| SSO 353382 | 400 | 55 | 66 |
| | 80 | 61 | 69 |
| | 16 | 85 | 73 |
| | 3.2 | 115 | 95 |
| | 0.64 | 112 | 106 |
| | 0.16 | 116 | 99 |

### Example 19

### Effect of SSO on SR-B1 mRNA Levels in the Presence of Vps25 and Vps36 Inhibitors

SSO 353382 was selected and tested independently in b.END cells in the presence and absence of Vps25 and Vps36 inhibitors. Vps25 and Vps36 are other members of the ESCRT pathway that may be involved in the functional uptake of SSOs. The SSO 353382 and negative control siRNA were described previously in Table 3.

Vps25 siRNA was a pool of four siRNAs targeted to Vps25 and are denoted as “Vps25 siRNA-1,” “Vps25 siRNA-2,” “Vps25 siRNA-3,” and “Vps25 siRNA-4.” The Vps25 and Vps36 siRNAs were purchased from Dharmacon Research Inc. (Boulder, Colo., USA) and are described in Table 34, below. The nucleosides are ribonucleosides (RNA) and the internucleoside linkages are phosphodiesters.

b.END cells were cultured in the same manner as described in Example 1. Cultured b.END cells were treated with Vps25 siRNA or with two different Vps36 siRNAs or negative control siRNA. Cells were plated at a density of 7,500 cells per well and transfected using Opti-MEM containing 5 μg/mL Lipofectamine 2000. First transfection was performed using 40 nM concentration of Vps25 siRNA, Vps36 siRNA or negative control siRNA. The siRNAs are denoted as “Vps25 siRNA” for Vps25 inhibitor; “Vps36 siRNA-1” or “Vps36 siRNA-2” for Vps36 inhibitors; and “Neg ctrl siRNA” for negative control. After a treatment period of 4 hours, transfection medium was replaced with complete growth medium and a second transfection was performed 24 hrs later in the same manner as described above. 24 hrs later SSO 353382 targeting SR-B1 was added to complete medium at concentrations listed in Table 35. RNA was isolated from cells after 24 hours and target mRNA levels were measured by qRT-PCR utilizing the method described in Example 1. The expression data was normalized to RIBOGREEN (Invitrogen) and mean values of three replicates are provided below.

As illustrated in Tables 35 and 36, an increase in reduction of SR-B1 mRNA levels was observed in b.END cells for SSO 353382 in the presence of Vps25 and Vps36 inhibitors as compared to the negative control. The results demonstrate that inhibition of Vps25 and Vps36 increases the potency of SSO 353382. As expected, treatment with Vps25 and Vps36 inhibitors reduced Vps25 and Vps36 mRNA levels in b.END cells.

TABLE 34
Vps25 and Vps36 siRNAs

| RNA | Oligo No. | Composition | SEQ ID No. |
|---|---|---|---|
| Vps25 siRNA-1 | ISIS 518462 | 5′-GAAUAAGUCUAGCUUCCUGTT-3′ | 72 |
| | ISIS XXXX01 | 5′-CAGGAACUAGACUUAUUCTT-3′ | 73 |
| Vps25 siRNA-2 | ISIS 518463 | 5′-GAAUAACUCUGUGUUUACUTT-3′ | 74 |
| | ISIS XXXX02 | 5′-AGUAAACACAGAGUUAUUCTT-3′ | 75 |
| Vps25 siRNA-3 | ISIS 518464 | 5′-GAAAGGGAACCUCGAGUGGTT-3′ | 76 |
| | ISIS XXXX03 | 5′-CCACUCGAGGUUCCCUUUCTT-3′ | 77 |
| Vps25 siRNA-4 | ISIS 518465 | 5′-UCAACAACGUCAAGCUACATT-3′ | 78 |
| | ISIS XXXX04 | 5′-UGUAGCUUGACGUUGUUGATT-3′ | 79 |
| Vps36 siRNA-1 | ISIS 505601 | 5′-UUAUUAGCGAUUGAUUUGGTT-3′ | 80 |
| | ISIS 505602 | 5′-CCAAAUCAAUCGCUAAUAATT-3′ | 81 |
| Vps36 siRNA-2 | ISIS 505603 | 5′-UUCUGAUAAACGCCUGUAATT-3′ | 82 |
| | ISIS 505604 | 5′-UUACAGGCGUUUAUCAGAATT-3′ | 83 |

TABLE 35
Effect of SSO on SR-B1 mRNA levels in the
presence of Vps25 inhibitor in b.END cells

| | Conc. of | SR-B1 mRNA level |
|---|---|---|
| Treatment | SSO (nM) | (% control) |
| Vps25 siRNA + | 10000 | 24 |
| SSO 353382 | 2000 | 31 |
| | 400 | 44 |
| | 80 | 56 |
| | 16 | 58 |
| | 3.2 | 83 |
| | 0.64 | 95 |
| | 0.16 | 112 |
| Con siRNA | 10000 | 41 |
| (neg control) + | 2000 | 53 |
| SSO 353382 | 400 | 66 |
| | 80 | 69 |
| | 16 | 73 |
| | 3.2 | 95 |
| | 0.64 | 106 |
| | 0.16 | 99 |

TABLE 36
Effect of SSO on SR-B1 mRNA levels in the
presence of Vps36 inhibitors in b.END cells

| | Conc. of | SR-B1 mRNA level |
|---|---|---|
| Treatment | SSO (nM) | (% control) |
| Vps36 siRNA-1 + | 10000 | 18.84 |
| SSO 353382 | 2000 | 20.35 |
| | 400 | 21.35 |
| | 80 | 28.84 |
| | 16 | 43.36 |
| | 3.2 | 59.89 |
| | 0.64 | 77.69 |
| | 0.16 | 81.41 |
| Vps36 siRNA-2 + | 10000 | 16.87 |
| SSO 353382 | 2000 | 18.41 |
| | 400 | 22.32 |
| | 80 | 29.52 |
| | 16 | 36.89 |
| | 3.2 | 50.79 |
| | 0.64 | 62.44 |
| | 0.16 | 63.75 |
| Con siRNA | 10000 | 40.88 |
| (neg control) + | 2000 | 53.17 |
| SSO 353382 | 400 | 66.17 |
| | 80 | 68.53 |
| | 16 | 72.93 |
| | 3.2 | 95.32 |
| | 0.64 | 105.72 |
| | 0.16 | 98.55 |

### Example 20

### Effect of SSO on SR-B1 mRNA Levels in the Presence of Vps4 Inhibitor

SSO 353382 was selected and tested in MHT and b.END cells in the presence and absence of Vps4 inhibitor. Vps4 is another member of the ESCRT pathway that may be involved in the functional uptake of SSOs. The SSO 353382 and negative control siRNA were described previously in Table 3.

The Vps4 siRNA was purchased from Dharmacon Research Inc. (Boulder, Colo., USA) and is described in Table 37, below. The nucleosides are ribonucleosides (RNA) and the internucleoside linkages are phosphodiesters.

MHT and b.END cells were cultured in the same manner as described in Example 1. Cultured MHT and b.END cells were treated with Vps4 siRNA or with a neg control siRNA targeting SR-B1. Cells were plated at a density of 7,500 cells per well and transfected using Opti-MEM containing 5 μg/mL Lipofectamine 2000. First transfection was performed using 40 nM concentration of Vps4 siRNA, or negative control siRNA. The siRNA is denoted as “Vps4 siRNA” for Vps4 inhibitor; and “Neg ctrl siRNA” for negative control. After a treatment period of 4 hours, transfection medium was replaced with complete growth medium and a second transfection was performed 24 hrs later in the same manner as described above. 24 hrs later SSO 353382 targeting SR-B1 was added to complete medium at the concentrations listed in Table 38. RNA was isolated from cells after 24 hours and target mRNA levels were measured by qRT-PCR utilizing the method described in Example 1. The expression data was normalized to RIBOGREEN (Invitrogen) and mean values of three replicates are provided below.

As illustrated in Table 38, an increase in reduction of SR-B1 mRNA levels was observed in MHT and b.END cells for SSO 353382 in the presence of Vps4 inhibitor as compared to the negative control. The results demonstrate that inhibition of Vps4 increases the potency of SSO 353382. As expected, treatment with Vps4 inhibitor reduced Vps4 mRNA levels in MHT and b.END cells.

TABLE 37
Vp4 siRNA

| | | SEQ ID |
|---|---|---|
| Oligo No. | Composition | No. |
| Vps4 siRNA | 5′-UAAAUUCCCACACUUAUUCTT-3′ | 84 |
| | 5′-GAAUAAGUGUGGGAAUUUATT-3′ | 85 |

TABLE 38
Effect of SSO on SR-B1 mRNA levels in the presence
of Vps4 inhibitor in MHT and b.END cells

| | |
|---|---|
| | SR-B1 mRNA level |
| Conc. of | (% control) |

| Treatment | SSO (nM) | MHT cells | b.END cells |
|---|---|---|---|
| Vps4 siRNA + | 10000 | 20 | 24 |
| SSO 353382 | 2000 | 35 | 33 |
| | 400 | 43 | 40 |
| | 80 | 42 | 50 |
| | 16 | 70 | 59 |
| | 3.2 | 88 | 76 |
| | 0.64 | 93 | 97 |
| | 0.16 | 107 | 115 |
| Neg ctrl siRNA + | 10000 | 26 | 41 |
| SSO 353382 | 2000 | 42 | 53 |
| | 400 | 55 | 66 |
| | 80 | 61 | 69 |
| | 16 | 85 | 73 |
| | 3.2 | 115 | 95 |
| | 0.64 | 112 | 106 |
| | 0.16 | 116 | 99 |

### Example 21

### Effect of SSO on SR-B1 mRNA Levels in the Presence of Lip5 Inhibitor

SSO 353382 was selected and tested in MHT and b.END cells in the presence and absence of Lip5 inhibitor. Lip5 is another member of the ESCRT pathway that may be involved in the functional uptake of SSOs. The SSO 353382 and negative control siRNA were described previously in Table 3.

The Lip5 siRNA was purchased from Dharmacon Research Inc. (Boulder, Colo., USA) and is described in Table 39, below. The nucleosides are ribonucleosides (RNA) and the internucleoside linkages are phosphodiesters.

MHT and b.END cells were cultured in the same manner as described in Example 1. Cultured MHT and b.END cells were treated with Lip5 siRNA or with a neg control siRNA targeting SR-B1. Cells were plated at a density of 7,500 cells per well and transfected using Opti-MEM containing 5 μg/mL Lipofectamine 2000. First transfection was performed using 40 nM concentration of Lip5 siRNA, or negative control siRNA. The siRNA is denoted as “Lip5 siRNA” for Lip5 inhibitor; and “Neg ctrl siRNA” for negative control. After a treatment period of 4 hours, transfection medium was replaced with complete growth medium and a second transfection was performed 24 hrs later in the same manner as described above. 24 hrs later SSO 353382 targeting SR-B1 was added to complete medium at the concentrations listed in Table 40. RNA was isolated from cells after 24 hours and target mRNA levels were measured by qRT-PCR utilizing the method described in Example 1. The expression data was normalized to RIBOGREEN (Invitrogen) and mean values of three replicates are provided below.

As illustrated in Table 40, an increase in reduction of SR-B1 mRNA levels was observed in MHT and b.END cells for SSO 353382 in the presence of Lip5 inhibitor as compared to the negative control. The results demonstrate that inhibition of Lip5 increases the potency of SSO 353382. As expected, treatment with Lip5 inhibitor reduced Lip5 mRNA levels in MHT and b.END cells (data not shown).

TABLE 39
Lip5 siRNA

| Oligo No. | Composition | SEQ ID No. |
|---|---|---|
| Lip5 siRNA | 5′-GCACAGGUGUAACGAGUAATT-3′ | 86 |
| | 5′-UUACUCGUUACACCUGUGCTT-3′ | 87 |

TABLE 40
Effect of SSO on SR-B1 mRNA levels in the presence of Lip5
inhibitor in MHT and b.END cells

| | |
|---|---|
| | SR-B1 mRNA level |
| Conc. of SSO | (% control) |

| Treatment | (nM) | MHT cells | b.END cells |
|---|---|---|---|
| Lip5 siRNA + | 10000 | 20 | 23 |
| SSO 353382 | 2000 | 23 | 24 |
| | 400 | 32 | 36 |
| | 80 | 43 | 47 |
| | 16 | 63 | 56 |
| | 3.2 | 91 | 70 |
| | 0.64 | 117 | 74 |
| | 0.16 | 117 | 96 |
| Neg ctrl siRNA + | 10000 | 26 | 23 |
| SSO 353382 | 2000 | 42 | 31 |
| | 400 | 55 | 43 |
| | 80 | 61 | 51 |
| | 16 | 85 | 76 |
| | 3.2 | 115 | 104 |
| | 0.64 | 112 | 79 |
| | 0.16 | 116 | 125 |

### Example 22

### Effect of SSOs on SR-B1 and Malat1 mRNA Levels in the Presence of Rab27 Inhibitors

SSO 353382 targeting SRB-1 and SSO 399479 targeting Malat1 were evaluated for functional uptake in MHT cells in the presence and absence of Rab27 inhibitors. Rab27A and Rab27B are members of the Rab family of small GTPases that control different steps of exosome release, including transport of multivesicular bodies and docking at the plasma membrane that may be involved in the functional uptake and secretion of SSOs. The SSO 353382 and negative control siRNA were described previously in Table 3.

SSO 399479 targeting Malat1 is a 5-10-5 MOE gapmer and was prepared using similar procedures reported in the literature (Egli et al., J. Am. Chem. Soc., 2011, 133(41), 16642-16649; and Pallan, et al., Chem. Com. (Camb), 2012, 48(66), 8195-8197). The Rab27A and Rab27B siRNAs were purchased from Ambion, Life Technologies (Carlsbad, Calif., USA). The SSO and siRNAs are described in Table 41, below. A subscript “s” between two nucleosides indicates a phosphorothioate internucleoside linkage. The absence of a subscript “s” between two nucleosides indicates a phosphodiester internucleoside linkage. Nucleosides without a subscript are ribonucleosides (RNA). Nucleosides with subscripts “d” are β-D-2′-deoxyribonucleosides. Nucleosides with subscripts “e” are 2′-O-methoxyethyl (MOE) modified nucleosides. ^{me}C indicates a 5-methylcytosine nucleoside.

MHT cells were cultured in the same manner as described in Example 1. Cultured MHT cells were treated with Rab27A siRNA, Rab27B or with a negative control siRNA. Cells were plated at a density of 7,500 cells per well and transfected using Opti-MEM containing 5 μg/mL Lipofectamine 2000. First transfection was performed using 40 nM concentration of Rab27A siRNA, Rab29B or negative control siRNA. The siRNAs are denoted as “Rab27A siRNA”, and “Rab27B siRNA” for Rab27A and Rab27B inhibitors. “Neg ctrl siRNA” indicates for negative control. After a treatment period of 4 hours, transfection medium was replaced with complete growth medium and a second transfection was performed 24 hrs later in the same manner as described. 24 hrs later SSO 353382 targeting SR-B1 and SSO 399479 targeting Malat1 were added to complete medium above at the concentrations listed in Tables 42 to 43a. RNA was isolated from cells after 24 hours and target mRNA levels were measured by qRT-PCR utilizing the method described in Example 1. The expression data was normalized to RIBOGREEN (Invitrogen) and mean values of three replicates are provided below.

As illustrated in Tables 42 to 43a, an increase in reduction of SR-B1 and Malat1 mRNA levels was observed in MHT cells for SSO 353382 and 399479 in the presence of Rab27 inhibitors as compared to the negative control. The results demonstrate that inhibition of Rab27A and Rab27B increases the potency of SSO 353382 and 399479. As expected, treatment with Rab27A and Rab27B inhibitors reduced Rab27A and Rab27B mRNA levels in MHT cells.

TABLE 41
SSO and Rab27 siRNAs targeting SR-B1 and Malat1

| | | SEQ |
|---|---|---|
| Oligo No. | Composition | ID No. |
| SSO 399479 | 5′-^{m}CₑₛGₑₛGₑₛTₑₛGₑₛ^{m}C_{ds}A_{ds}A_{ds}G_{ds}G_{ds} | 46 |
| | ^{m}C_{ds}T_{ds}T_{ds}A_{ds}G_{ds}GₑₛAₑₛAₑₛTₑₛTₑ-3′ | |
| Rab27A | 5′-GGAGAGGUUUCGUAGCUUATT-3′ | 88 |
| siRNA | 5′-UAAGCUACGAAACCUCUCCTG-3′ | 89 |
| Rab27B | 5′-CAGAGCUUCUUGAAUGUCATT-3′ | 90 |
| siRNA | 5′-UCACAUUCAAGAAGCUCUGTT-3′ | 91 |

TABLE 42
Effect of SSO 353382 on SR-B1 mRNA levels in the
presence of Rab27A inhibitorin MHT cells

| | Conc. of SSO | SR-B1 mRNA level |
|---|---|---|
| Treatment | 353382 (nM) | (% control) |
| Rab27A siRNA + | 2000 | 30 |
| SSO 353382 | 400 | 35 |
| | 80 | 46 |
| | 16 | 78 |
| Neg ctrl siRNA + | 2000 | 41 |
| SSO 353382 | 400 | 62 |
| | 80 | 74 |
| | 16 | 94 |

TABLE 42a
Effect of SSO 353382 on SR-B1 mRNA levels in the
presence of Rab27B inhibitor in MHT cells

| | Conc. of SSO | SR-B1 mRNA |
|---|---|---|
| Treatment | 353382 (nM) | level (% control) |
| Rab27B siRNA + | 2000 | 23 |
| SSO 353382 | 400 | 29 |
| | 80 | 36 |
| | 16 | 53 |
| Neg ctrl siRNA + | 2000 | 38 |
| SSO 353382 | 400 | 56 |
| | 80 | 66 |
| | 16 | 92 |

TABLE 43
Effect of SSO 399479 on Malat1 mRNA levels in the
presence of Rab27A inhibitor in MHT cells

| | Conc. of SSO | Malat1 mRNA level |
|---|---|---|
| Treatment | 399479 (nM) | (% control) |
| Rab27A siRNA + | 100 | 12.9 |
| SSO 399479 | 20 | 22.25 |
| | 4 | 36.47 |
| | 0.8 | 50.27 |
| Neg ctrl siRNA + | 100 | 12.54 |
| SSO 399479 | 20 | 28.94 |
| | 4 | 58.38 |
| | 0.8 | 63.07 |

TABLE 43a
Effect of SSO 399479 on Malat1 mRNA levels in the
presence of Rab27B inhibitor in MHT cells

| | Conc. of SSO | Malat1 mRNA |
|---|---|---|
| Treatment | 399479 (nM) | level (% control) |
| Rab27B siRNA + | 100 | 15 |
| SSO 399479 | 20 | 26 |
| | 4 | 61 |
| | 0.8 | 66 |
| Neg ctrl siRNA + | 100 | 20 |
| SSO 399479 | 20 | 58 |
| | 4 | 95 |
| | 0.8 | 94 |

### Example 23

### Effect of SSOs on SR-B1 and Malat1 mRNA Levels in the Presence of SYTL4 and SLAC2B Inhibitors

SSO 353382 targeting SR-B1 and SSO 399479 targeting Malat1 were tested in MHT cells in the presence and absence of SYTL4 and SLAC2B inhibitors. SYTL4 and SLAC2B are Rab27 effectors that might play a role in SSO secretion through exosome. The SSO 353382, 399479 and negative control siRNA were described previously in Tables 3 and 41.

The SYTL4 and SLAC2B siRNAs were purchased from Dharmacon Research Inc. (Boulder, Colo., USA) and is described in Table 44, below. The nucleosides are β-D-2′-deoxyribonucleosides and the internucleoside linkages are phosphodiesters.

MHT cells were cultured in the same manner as described in Example 1. Cultured MHT cells were treated with two different SYTL4 siRNAs, SLAC2B siRNAs or with a negative control siRNA. Cells were plated at a density of 7,500 cells per well and transfected using Opti-MEM containing 5 μg/mL Lipofectamine 2000. First transfection was performed using 40 nM concentration of SYTL4 and SLAC2B siRNAs or negative control siRNA. The siRNAs are denoted as “SYTL4-1 siRNA”, “SYTL4-2 siRNA”, “SLAC2B-1 siRNA”, or “SLAC2B-2 siRNA” for SYTL4 and SLAC2B inhibitors. “Neg ctrl siRNA” indicates for negative control. After a treatment period of 4 hours, transfection medium was replaced with complete growth medium and a second transfection was performed 24 hrs later in the same manner as described above. 24 hrs later SSO 353382 targeting SR-B1 and SSO 399479 targeting Malat1 were added to complete medium at the concentrations listed in Tables 45 to 46a. RNA was isolated from cells after 24 hours and target mRNA levels were measured by qRT-PCR utilizing the method described in Example 1. The expression data was normalized to RIBOGREEN (Invitrogen) and mean values of three replicates are provided below.

As illustrated in Tables 45 to 46a, an increase in reduction of SR-B1 and Malat1 mRNA levels was observed in MHT cells for SSO 353382 and 399479, respectively, in the presence of SYTL4 and SLAC2B inhibitors as compared to the negative control. The results demonstrate that inhibition of SYTL4 and SLAC2B increases the potency of SSO 353382 and 399479.

TABLE 44
SYTL4 and SLAC2B siRNAs targeting SR-B1 and Malat1

| siRNA | Oligo No. | Composition | SEQ ID No. |
|---|---|---|---|
| SYTL4-1 siRNA | ISIS 505577 | 5′-GAAGAGUCCAGAUUUAUCCTT-3′ | 92 |
| | ISIS 505578 | 5′-GGAUAAAUCUGGACUCUUCTT-3′ | 93 |
| SYTL4-2 siRNA | ISIS 505579 | 5′-CCAAUCUCCAGUUGCUUUCTT-3′ | 94 |
| | ISIS 505580 | 5′-GAAAGCAACUGGAGAUUGGTT-3′ | 95 |
| SLAC2B-1 | ISIS 505581 | 5′-UUGGUCGUAGGUUCUUCUCTT-3′ | 96 |
| siRNA | ISIS 505582 | 5′-GAGAAGAACCUACGACCAATT-3′ | 97 |
| SLAC2B-2 | ISIS 505583 | 5′-UUCCGUACAGUUUCUUAUUTT-3′ | 98 |
| siRNA | ISIS 505584 | 5′-AAUAAGAAACUGUACGGAATT-3′ | 99 |

TABLE 45
Effect of SSO 353382 on SR-B1 mRNA levels in the
presence of SYTL4 inhibitors

| | Conc. of SSO | SR-B1 mRNA level |
|---|---|---|
| Treatment | 353382 (nM) | (% control) |
| SYTL4-1 siRNA + | 2000 | 28 |
| SSO 353382 | 400 | 36 |
| | 80 | 41 |
| | 16 | 55 |
| SYTL4-2 siRNA + | 2000 | 24 |
| SSO 353382 | 400 | 38 |
| | 80 | 49 |
| | 16 | 77 |
| Neg ctrl siRNA + | 2000 | 39 |
| SSO 353382 | 400 | 55 |
| | 80 | 70 |
| | 16 | 82 |

TABLE 45a
Effect of SSO 353382 on SR-B1 mRNA levels in the
presence of SLAC2B inhibitors

| | Conc. of SSO | SR-B1 mRNA |
|---|---|---|
| Treatment | 353382 (nM) | level (% control) |
| SLAC2B-1 | 2000 | 21 |
| siRNA + | 400 | 26 |
| SSO 353382 | 80 | 33 |
| | 16 | 45 |
| SLAC2B-2 | 2000 | 28 |
| siRNA + | 400 | 40 |
| SSO 353382 | 80 | 50 |
| | 16 | 63 |
| Neg ctrl siRNA + | 2000 | 39 |
| SSO 353382 | 400 | 55 |
| | 80 | 70 |
| | 16 | 82 |

TABLE 46
Effect of SSO 399479 on Malat1 mRNA levels in the
presence of SYTL4 inhibitors

| | Conc. of SSO | Malat1 mRNA level |
|---|---|---|
| Treatment | 399479 (nM) | (% control) |
| SYTL4-1 siRNA + | 100 | 21 |
| SSO 399479 | 20 | 49 |
| | 4 | 100 |
| | 0.8 | 101 |
| SYTL4-2 siRNA + | 100 | 17 |
| SSO 399479 | 20 | 29 |
| | 4 | 58 |
| | 0.8 | 63 |
| Neg ctrl siRNA + | 100 | 32 |
| SSO 399479 | 20 | 61 |
| | 4 | 93 |
| | 0.8 | 102 |

TABLE 46a
Effect of SSO 399479 on Malat1 mRNA levels in the
presence of SLAC2B inhibitors

| | Conc. of SSO | Malat1 mRNA level |
|---|---|---|
| Treatment | 399479 (nM) | (% control) |
| SLAC2B-1 | 100 | 23 |
| siRNA + | 20 | 34 |
| SSO 399479 | 4 | 66 |
| | 0.8 | 84 |
| SLAC2B-2 | 100 | 20 |
| siRNA + | 20 | 43 |
| SSO 399479 | 4 | 72 |
| | 0.8 | 105 |
| Neg ctrl siRNA + | 100 | 32 |
| SSO 399479 | 20 | 61 |
| | 4 | 93 |
| | 0.8 | 102 |

### Example 24

### Effect of SSO on SR-B1 mRNA Levels in the Presence of LDLR and AP2M1 Inhibitors

The effect of SSO 353382 on SRB-1 mRNA levels was evaluated in the presence and absence of LDLR and AP2M1 inhibitors. The SSO 353382 and negative control siRNA were described previously in Table 3.

LDLR was a pool of four siRNAs targeted to LDLR and are denoted as “LDLR siRNA-1,” “LDLR siRNA-2,” “LDLR siRNA-3,” and “LDLR siRNA-4.” The LDLR and AP2M1 siRNAs were purchased from Dharmacon Research Inc. (Boulder, Colo., USA) and are described in Tables 21 and 47. The nucleosides are ribonucleosides (RNA) and the internucleoside linkages are phosphodiesters.

MHT cells were cultured in the same manner as described in Example 1. Cultured MHT cells were transfected with LDLR siRNA, AP2M1 siRNA and negative control siRNA. LDLR siRNA comprises a mixture of four siRNAs as shown previously in Table 21. Cells were plated at a density of 7,500 cells per well and transfected using Opti-MEM containing 5 μg/mL Lipofectamine 2000. First transfection was performed using 50 nM concentration LDLR siRNA, AP2M1 siRNA or negative control siRNA. After a treatment period of 4 hrs, transfection medium was replaced with complete growth medium. 48 hrs later, SSO 353382 targeting SR-B1 was added to complete growth medium (DMEM, 10% FBS) at concentrations listed in Table 48. RNA was isolated from cells after 24 hours and SRB-1 mRNA levels were measured by qRT-PCR as described in Example 1. The expression data was normalized to RIBOGREEN (Invitrogen) and the mean results from three replicates are presented in Table 48, below.

As illustrated in Table 48, inhibition of LDLR and AP2M1 decreases the potency of SSO 353382 targeting SR-B1 compared to the negative control.

TABLE 47
AP2M1 siRNA

| | | | SEQ |
|---|---|---|---|
| | | | ID |
| siRNA | Oligo No. | Composition | No. |
| AP2M1 | ISIS 417468 | 5′-GAUCGGAGGCUUAUUCAUCTT-3′ | 100 |
| siRNA | ISIS 421364 | 5′-GAUGAAUAAGCCUCCGAUCTT-3′ | 101 |

TABLE 48
Effect of SSO on SR-B1 mRNA levels in the presence
of LDLR and AP2M1 inhibitors

| | Conc. of | SR-B1 mRNA |
|---|---|---|
| Treatment | SSO (nM) | level (% control) |
| LDLR siRNA + | 10000 | 39 |
| SSO 353382 | 2000 | 54 |
| | 400 | 56 |
| | 80 | 57 |
| | 16 | 74 |
| | 3.2 | 95 |
| | 0.64 | 98 |
| | 0.16 | 92 |
| AP2M1 siRNA + | 10000 | 41 |
| SSO 353382 | 2000 | 56 |
| | 400 | 61 |
| | 80 | 60 |
| | 16 | 95 |
| | 3.2 | 103 |
| | 0.64 | 117 |
| | 0.16 | 108 |
| Neg ctrl siRNA + | 10000 | 33 |
| SSO 353382 | 2000 | 35 |
| | 400 | 39 |
| | 80 | 41 |
| | 16 | 55 |
| | 3.2 | 73 |
| | 0.64 | 71 |
| | 0.16 | 79 |

### Example 25

### Effect of SSO on SR-B1 mRNA Levels in the Presence of LDLR and AP2M1 Inhibitors

The effect of SSO 353382 on SRB-1 mRNA levels was evaluated in the presence or absence of LDLR and AP2M1 inhibitors. The SSO 353382 and negative control siRNA were described previously in Table 3.

LDLR was a pool of four siRNAs targeted to LDLR and are denoted as “LDLR siRNA-1,” “LDLR siRNA-2,” “LDLR siRNA-3,” and “LDLR siRNA-4.” The LDLR and AP2M1 siRNA were purchased from Dharmacon Research Inc. (Boulder, Colo., USA) and are described previously in Tables 21 and 47.

MHT cells were cultured in the same manner as described in Example 1. Cultured MHT cells were transfected with LDLR siRNA, AP2M1 siRNA and negative control siRNA. LDLR siRNA comprises a mixture of four siRNAs as shown previously. Cells were plated at a density of 7,500 cells per well and transfected using Opti-MEM containing 5 μg/mL Lipofectamine 2000. First transfection was performed with a series of concentrations of LDLR siRNA, AP2M1 siRNA or negative control siRNA as presented in Table 49, below. After a treatment period of 4 hrs, transfection medium was replaced with complete growth medium. 24 hrs later transfection with siRNAs was repeated as described above. 24 hrs later, SSO 353382 targeting SR-B1 was added at 2 μM to complete growth medium (DMEM, 10% FBS). RNA was isolated from cells after 24 hours and SRB-1 mRNA levels were measured by qRT-PCR as described in Example 1. The expression data was normalized to RIBOGREEN (Invitrogen) and the mean results from three replicates are presented in the table below.

As illustrated in Table 49, inhibition of target reduction was siRNA dose-dependent. Consistent with our previous results, inhibition of LDLR and AP2M1 decreases the potency of SSO 353382 compared to the negative control.

TABLE 49
Dose-response study of LDLR and AP2M1 siRNAs
in the presence of SSO 353382 on SR-B1 mRNA levels

| | Conc. of | SR-B1 mRNA |
|---|---|---|
| Treatment | siRNA (nM) | level (% control) |
| LDLR siRNA + | 75 | 66.6 |
| SSO 353382 | 37.5 | 63.1 |
| | 18.375 | 50.2 |
| | 9.375 | 54.2 |
| | 4.6 | 47.0 |
| | 2.3 | 34.0 |
| | 1.17 | 31.5 |
| | 0.58 | 32.6 |
| AP2M1 siRNA + | 75 | 60.1 |
| SSO 353382 | 37.5 | 54.4 |
| | 18.375 | 41.3 |
| | 9.375 | 42.2 |
| | 4.6 | 38.5 |
| | 2.3 | 33.1 |
| | 1.17 | 32.6 |
| | 0.58 | 32.4 |
| Neg ctrl siRNA + | 75 | 33.6 |
| SSO 353382 | 37.5 | 2 |
| | 18.375 | 25.2 |
| | 9.375 | 26.8 |
| | 4.6 | 33.9 |
| | 2.3 | 28.9 |
| | 1.17 | 28.4 |
| | 0.58 | 29.2 |

### Example 26

### Evaluation of SSO Secretion in Multivesicular Bodies

The secretion of SSO 353382 in multivesicular bodies was evaluated. The SSO 353382 was previously described in Table 3.

MHT cells were cultured in the same manner as described in Example 1. Cultured MHT cells were plated at a density of 7,500 cells per well and transfected using Opti-MEM containing 5 μg/mL Lipofectamine 2000. SSO 353382 was added at 10 uM concentration to complete growth medium (DMEM, 10% FBS). After 24 hrs, cells were washed and incubated for 48 hrs. Secreted exosomes were isolated from medium and SSO was detected with a SSO antibody. The results obtained showed that the exosomes contained the SSO compared to untreated control that did not have any SSOs (data not shown).

### Example 27

### Evaluation of SSO Uptake and Secretion in Vps28 Depleted Cells Using ³H-Labeled SSO

The uptake and secretion of SSO 353382 in MHT cells were evaluated using ³H-labeled SSO. The SSO 353382 and siRNAs were previously described in Table 3.

MHT cells were cultured in the same manner as described in Example 1. Cultured MHT cells were transfected with Vps28 siRNA-1 and negative control siRNA. Cells were plated at a density of 46,000 cells per 24-well and transfected using Opti-MEM containing 5 μg/mL Lipofectamine 2000. First transfection was performed with 75 nM concentration of Vps28 siRNA-1 and negative control siRNA. After a treatment period of 4 hrs, transfection medium was replaced with complete growth medium and a second transfection was performed 24 hrs later in the same manner as above. 24 hrs later ³H-labeled SSO 353382 was added at 400 nM concentration to complete growth medium (DMEM, 10% FBS). Radioactivity of cells was measured at various time intervals as indicated in Table 50, below. As illustrated, the uptake of SSO into cells was reaching a plateau after 1 hr in both negative control and Vps28 siRNA-1 treated cells. As shown with the fluorescent SSO, the radioactive SSO accumulation was higher in Vps28 depleted cells.

To evaluate the secretion of SSO into the medium, cells were incubated with ³H-labeled SSO 353382 for 24 hrs. Cells were then washed and the release of SSO into the medium was measured over various time intervals as indicated in Table 51, below. As illustrated, the SSO gets released very quickly reaching a plateau after about 40 min. The secretion of SSO in Vps28 depleted cells is higher than in control siRNA treated cells.

TABLE 50
SSO 353382 uptake in Vps28 depleted cells

| Treatment | Time (hr) | Total cpm in cell lysate |
|---|---|---|
| Vps28 siRNA-1 + | 0.5 | 11530 |
| SSO 353382 | 1 | 350990 |
| | 2 | 282930 |
| | 6.5 | 288000 |
| | 24 | 300650 |
| Neg ctrl siRNA + | 0.5 | 6470 |
| SSO 353382 | 1 | 197750 |
| | 2 | 196890 |
| | 6.5 | 236160 |
| | 24 | 170130 |

TABLE 51
SSO 353382 release into medium in Vps28 depleted cells

| Treatment | Time | | Total SSO secretion (cpm) |
|---|---|---|---|
| Vps28 siRNA-1 + | 5 | min | 32440 |
| SSO 353382 | 40 | min | 57660 |
| | 1.25 | hr | 72820 |
| | 2 | hr | 63940 |
| | 3 | hr | 66060 |
| | 5 | hr | 62190 |
| | 6 | hr | 62120 |
| | 8 | hr | 73410 |
| | 30 | hr | 104590 |
| Neg ctrl siRNA + | 5 | min | 17570 |
| SSO 353382 | 40 | min | 34360 |
| | 1.25 | hr | 38820 |
| | 2 | hr | 36380 |
| | 3 | hr | 35080 |
| | 5 | hr | 35250 |
| | 6 | hr | 31870 |
| | 8 | hr | 40990 |
| | 30 | hr | 48320 |

### Example 28

### Evaluation of SSO Accumulation in Vps28 Depleted Cells

To evaluate if secreted SSO 353382 can be taken up by cells, MHT cells were plated in Transwell chambers and transfected with Vps28 siRNA-1 or negative control siRNA. The SSO 353382 and siRNAs were described previously in Table 3.

Cells were washed and top chamber with SSO treated cells (donor) was placed on cells that did not receive the SSO (recipient). After 24 hrs of treatment, SSO accumulation in donor and recipient cells was measured with a FacsCalibur following standard procedures. Results are presented in Table 52, below.

As illustrated, the cells indeed took up the secreted SSO. The acceptor cells accumulated more SSO from the Vps28 siRNA treated donor cells than the negative control siRNA treated donor cells. These results show that secreted SSO can be taken up by cells. In addition, it shows that Vps28 siRNA treated cells take up more SSOs.

TABLE 52
SSO accumulation in Vps28 depleted cells

| | | SSO accumulation |
|---|---|---|
| Treatment | Cells | (% neg control) |
| Vps28 siRNA | recipient | 139 |
| Vps28 siRNA + | donor | 25 |
| SSO 353382 | | |
| Neg ctrl siRNA | recipient | 100 |
| Neg ctrl siRNA + | donor | 13 |
| SSO 353382 | | |

### Example 29

### Evaluation of SSO Accumulation in the Nuclei of Vps28 Depleted MHT Cells

To evaluate the accumulation of SSO in the nuclei of Vps28 depleted cells, MHT cells were plated at a density of 200,000 cells per 35 mm dish (collagen-coated glass bottom culture dishes from MatTek) and transfected using Opti-MEM containing 5 μg/mL Lipofectamine 2000. First transfection was performed using 50 nM concentration of Vsp28 siRNA or negative control. After a treatment period of 4 hrs, transfection medium was replaced with complete growth medium and a second transfection was performed 24 hrs later in the same manner as described above. An AF-488 conjugated SSO 353382 (also known as SSO 407988) was added to complete growth medium (DMEM, 10% FBS) at 400 nM concentration. After 24 hrs, fluorescence intensity in nuclei was measured on a confocal microscope (Olympus FV1000). Results are presented in Table 53, below. As illustrated, an increase in SSO accumulation in the nuclei of Vps28 siRNA treated cells was observed as compared to the negative control. Fluorescence intensity of negative control siRNA treated cells in nuclei was 12 units, while the Vps28 siRNA treated cells was 174 units.

TABLE 53
SSO in nuclei of Vps28 depleted cells

| | Fluorescence intensity |
|---|---|
| Treatment | (units) |
| Vps28 siRNA-1 + | 174 |
| SSO 353382 | |
| Neg ctrl siRNA + | 12 |
| SSO 353382 | |

We claim:

1.-168. (canceled)

169. A method of sensitizing a cell for antisense modulation comprising, reducing the amount or activity of at least one ESCRT associated nucleic acid transcript; and thereby sensitizing the cell for antisense modulation.

170. The method of claim 169 comprising contacting the cell with at least one ESCRT modulator.

171. The method of claim 170, wherein at least one ESCRT modulator is an ESCRT-I modulator.

172. The method of claim 170, wherein at least one ESCRT modulator is a Vps28 modulator.

173. The method of claim 170, wherein at least one ESCRT modulator is a Tsg101 modulator.

174. The method of any of claim 170, wherein at least one ESCRT modulator is a Vps37 modulator.

175. The method of any of claim 170, wherein at least one ESCRT modulator is an Mvb12 modulator.

176. The method of claim 170, wherein at least one ESCRT modulator is an Mvb12a modulator.

177. The method of claim 170, wherein at least one ESCRT modulator is an Mvb12b modulator.

178. The method of any of claim 170, wherein at least one ESCRT modulator is an Hrs modulator.

179. The method of any of claim 170, wherein at least one ESCRT modulator is an Alix modulator.

180. The method of any of claim 170, wherein at least one ESCRT modulator is an ESCRT-II modulator.

181. The method of any of claim 170, wherein at least one ESCRT modulator is Vps4 modulator.

182. The method of any of claim 170, wherein at least one ESCRT modulator is selected from among: a Vps22 modulator, a Vps36 modulator, a Vps4, and a Vps25 modulator.

183. The method of any of claim 170, wherein at least one ESCRT modulator is an ESCRT-III modulator.

184. The method of claim 182, wherein the antisense compound targeting an ESCRT transcript is single-stranded.

185. The method of claim 182, wherein the antisense compound targeting an ESCRT transcript is double-stranded.

186. The method of claim 182, wherein the antisense compound targeting an ESCRT transcript is an RNAi compound.

187. The method of claim 182, wherein the antisense compound targeting an ESCRT transcript is an RNase H antisense compound.

188. The method of claim 169, wherein the antisense compound complementary to a target nucleic acid other than an ESCRT transcript comprises at least one conjugate.
